(19) United States
(12) Patent Application Publication
(10) Pub. No.: US 2013/0310374 A1
(43) Pub. Date: Nov. 21, 2013

(54) Substituted Imidazoquinoline Derivatives

(71) Applicants: Sanjay Kumar, Mumbai (IN); Rajiv Sharma, Mumbai (IN); Vijaykumar Bhagwan Deore, Mumbai (IN); Nilambari Nilkanth Yewalkar, Mumbai (IN); Veena R. Agarwal, Mumbai (IN); Nilesh Dagia, Mumbai (IN); Nishigandha Naik, Mumbai (IN)

(72) Inventors: Sanjay Kumar, Mumbai (IN); Rajiv Sharma, Mumbai (IN); Vijaykumar Bhagwan Deore, Mumbai (IN); Nilambari Nilkanth Yewalkar, Mumbai (IN); Veena R. Agarwal, Mumbai (IN); Nilesh Dagia, Mumbai (IN); Nishigandha Naik, Mumbai (IN)

(73) Assignee: PIRAMAL ENTERPRISES LIMITED`, Mumbai (IN)

(21) Appl. No.: 13/991,841

(22) PCT Filed: Dec. 5, 2011
(86) PCT No.: PCT/IB11/55449

§ 371 Date: Jun. 5, 2013

Related U.S. Application Data
(60) Provisional application No. 61/420,205, filed on Dec. 6, 2010.

(51) Int. Cl. C07D 471/04

(52) U.S. Cl. CPC C07D 471/04
USPC 514/232.8; 546/82; 514/293; 544/310; 514/274; 544/316; 544/126; 544/361; 514/253.03

(57) ABSTRACT

The present invention relates to substituted imidazo[4,5-c]quinoline derivatives of formula (I), wherein R₁, R₂ and R₃ are as defined in the specification, processes for their preparation, pharmaceutical compositions comprising compounds of the present invention and their use in the treatment of diseases or disorders mediated by one or more kinases, particularly proliferative diseases or disorders such as cancer. These compounds can also be used in the treatment of inflammation and angiogenesis related disorders.

### FIELD OF THE INVENTION

The present invention relates to substituted imidazo[4,5-c]quinoline derivatives, processes for their preparation, pharmaceutical compositions comprising compounds of the present invention and their use in the treatment of diseases or disorders mediated by one or more kinases, particularly proliferative diseases or disorders such as cancer. These compounds can also be used in the treatment of inflammatory diseases or disorders and angiogenesis related diseases or disorders.

### BACKGROUND OF THE INVENTION

Cancer can be defined as an abnormal growth of tissues characterized by a loss of cellular differentiation. It is caused due to a deregulation of the signaling pathways involved in cell survival, cell proliferation and cell death.

Angiogenesis is the process of forming new blood vessels and is critical in many normal and abnormal physiological states. Angiogenesis is normally observed in wound healing, fetal and embryonic development and formation of corpus luteum, endometrium and placenta. However, angiogenesis is also the fundamental step in the transition of tumors from a dormant state to a malignant state. In diseases like cancer, the body loses the ability to maintain balanced angiogenesis. New blood vessels feed diseased tissues, destroying normal tissues and sometimes are involved in tumor metastasis. Hence anti-angiogenic agents are a very promising class of drugs to block or slow the cancer growth.

Vascular Endothelial Growth Factor (VEGF), a signal protein, stimulates the growth of new blood vessels. It is involved in both vasculogenesis (the de novo formation of the embryonic circulatory system) and angiogenesis (the growth of blood vessels from pre-existing vasculature). Anti-VEGF therapies are important in the treatment of age-related macular degeneration and in certain cancers such as breast cancer, oesophageal cancer, melanoma, colorectal cancer and tumors of central nervous system.

Protein kinases play important roles in regulating most cellular functions such as proliferation, cell cycle, cell metabolism, survival, apoptosis, DNA damage repair, cell motility and response to the microenvironment. Protein kinases can be divided into broad groups based upon the identity of the amino acid(s) that they target (serine/threonine, tyrosine, lysine, and histidine). There are also dual-specific protein kinases that target both tyrosine and serine/threonine, such as Mitogen-Activated Protein Kinases (MAPKs). MAPKs are commonly activated in cancer cells and are known to contribute to tumorigenesis. The protein tyrosine kinases (PTKS) comprise a large family of kinases that regulate cell to cell signals involved in growth, differentiation, adhesion, motility, and death. Members of the tyrosine kinase include, but are not limited to, Muscle-Specific Receptor Tyrosine Kinase (MuSK), Janus kinase 2 (JAK2) and Reactive Oxygen Species (ROS). The JAKs are integral in signaling from extracellular cytokines, including the interleukins, interferons, as well as numerous hormones. The importance of these kinases in cellular survival is made evident by the fact that the loss of JAKs is often accompanied by immunodeficiency and non-viability in animal models.

The family of serine/threonine kinases includes, but is not limited to, DNA-dependent protein kinase (DNA-PK), activin receptor-like kinase 1 (ALK1), activin receptor-like kinase 1 (ALK2), CDC-like kinase 1 (CLK1), CDC-like kinase 4 (CLK4) and receptor-interacting serine/threonine-protein kinase 2 (RIPK2). The DNA-PK is a nuclear serine/threonine protein kinase that is activated upon association with DNA. DNA-PK has been shown to be a crucial component of both the DNA double-strand break (DSB) repair machinery and the V(D)J recombination apparatus. DNA-PK is required for the non-homologous end joining (NHEJ) pathway of DNA repair, which rejoins double-strand breaks. Hence DNA-PK finds use in the treatment of cancers. Aberrant activity of ALK (Activin Like Kinase) is involved in the development of brain tumors and over expression of ALK has been reported in neuroblastomas and several cell lines derived from neural tissue. ALK mediated signaling could play a role in the development and/or progression of a number of common solid tumors (J. Cell. Physiol., 2004, 199(3), 330-58).

ALK-1 is a type I cell surface receptor for transforming growth factor beta receptor type I (TGF-β1). Mutations in ALK-1 are associated with heredity hemorrhagic telangiectesia (HHT), suggesting a critical role for ALK-1 in the control of blood vessel development or repair (J. Med. Genet., 2003, 40, 494-502). Also, in-vivo experiments on ALK-1 knockout mice provide the evidence of ALK-1 involvement in angiogenesis (Proc. Natl. Acad. Sci, 2000, 97, 2626-2631).

Phosphatidylinositol-3-kinases or phosphoinositol-3-kinase (PI3-kinases or PI3Ks), are a family of lipid kinases that are capable of phosphorylating the 3 position hydroxyl group of the inositol ring of phosphatidylinositol. The PI3K family is composed of Class I, II and III. The classification is based on primary structure, regulation and in vitro lipid substrate specificity. Class III PI3K enzymes phosphorylate PI (phosphaotidylinositol) alone while, Class II PI3K enzymes phosphorylate both PI and PI 4-phosphate [PI(4)P]. Class I PI3K enzymes phosphorylate PI, PI(4)P and PI 4,5-biphosphate [PI(4, 5)P₂]. Class I PI3Ks are further divided into two groups, class Ia and class Ib, in terms of their activation mechanism. Class Ia PI3Ks include PI3K p110α, p110β and p110δ subtypes and are generally activated in response to growth factor-stimulation of receptor tyrosine kinases.

PI3K mediated signaling pathway plays a very important role in cancer cell survival, cell proliferation, angiogenesis and metastasis. Activation of PI3K results in a disturbance of control of cell growth and survival, and hence this pathway is an attractive target for the development of novel anticancer agents (Nat. Rev. Drug Discov., 2005, 4, 988-1004). Activation of PI3K results in the recruitment and activation of protein kinase B (AKT) onto the membrane, which gets phosphorylated at Serine 473 (Ser-473).

AKT is known to positively regulate cell growth (accumulation of cell mass) by activating the mTOR serine threonine kinase. Mammalian target of rapamycin (mTOR) serves as a molecular sensor that regulates protein synthesis on the basis of nutrients. mTOR regulates biogenesis by phosphorylating and activating p70S6 kinase (S6K1), which in turn enhances translation of mRNAs that have polypyrimidine tracts. The phosphorylation status of S6K1 is a bonafide read-out of mTOR function. Most tumors have an aberrant PI3K pathway (Nat. Rev. Drug Discov., 2005, 4, 988-1004). Since mTOR lies immediately downstream of PI3K, these tumors also have hyperactive mTOR function. Thus, most of the cancer types will potentially benefit from molecules that target PI3K and mTOR pathways.

Inhibition of PI3K-Akt pathway suppresses coagulation and inflammation (Arteriosclerosis, Thrombosis, and Vascular Biology, 2004, 24, 1963). Hence the compounds that are PI3K and/or mTOR inhibitors, find use in the treatment of cancers, autoimmune and inflammatory diseases and disorders.

Several proinflammatory cytokines, especially TNF-α (Tumor Necrosis Factor-α) and interleukins (IL-1β, IL-6, IL-8) play an important role in the inflammatory process. An increase in TNF-α synthesis/release is a common phenomenon during the inflammatory process. Inflammation is an inherent part of various disease states like rheumatoid arthritis, Crohn's disease, ulcerative colitis, septic shock syndrome, atherosclerosis, among other clinical conditions.

TNF-α has been implicated as a mediator in several diseases such as inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, allergic asthma, septic shock, endotoxic shock, atherosclerosis, ischemia-reperfusion injury, multiple sclerosis, sepsis, chronic recurrent uveitis, hepatitis C virus infection, malaria, ulcerative colitis and the like. Much research has been conducted to study the effect of TNF-α and anti-TNF-α therapies. Studies in the area of cancer have shown that with TNF-α therapy it is important to balance the cytotoxicity and systemic toxicity of the potential drug candidates.

GDC-0941 (Piramed Ltd. and Genentech Inc.) is a PI3K inhibitor and is in phase I clinical trials. BEZ-235 and BGT-226 (Novartis AG), both in phase I/II clinical trials, inhibit all isoforms of PI3K and also inhibit the kinase activity of mTOR. XL-765 (Exelixis Inc.) is also a dual inhibitor of mTOR and PI3K. The compound is in phase I clinical trials as an oral treatment for solid tumors.

PCT publications WO2006/122806 and WO2010139747 describe imidazoquinoline compounds as lipid and/or protein kinase inhibitors for the treatment of lipid and/or protein kinase dependent disease.

### SUMMARY OF THE INVENTION

According to one aspect of the present invention there are provided compounds of formula (I),

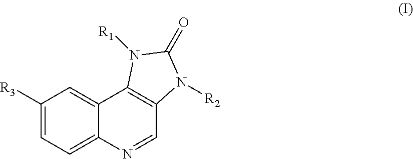

or stereoisomers, tautomers, polymorphs, prodrugs, N-oxides, pharmaceutically acceptable salts or solvates thereof.

According to another aspect of the present invention there are provided processes for preparing compounds of formula (I).

According to another aspect of the present invention there are provided novel intermediates useful for preparing compounds of formula (I).

According to another aspect of the present invention there is provided a method for inhibiting activity of a kinase selected from PI3K, mTOR, ALK-1 or ALK-2 comprising contacting the kinase with an effective amount of a compound of formula (I).

According to another aspect of the present invention there is provided a method for the treatment of proliferative diseases or disorders in a subject, comprising administering to the subject a therapeutically effective amount of a compound of formula (I) or stereoisomers, tautomers, N-oxides, pharmaceutically acceptable salts or solvates thereof.

According to another aspect of the present invention there is provided a method for the treatment of proliferative diseases or disorders mediated by one or more kinases selected from PI3K, mTOR, ALK-1 or ALK-2 in a subject, comprising administering to the subject in need thereof a therapeutically effective amount of a compound of formula (I) or a stereoisomer, a tautomer, a polymorph, a prodrug, an N-oxide, a pharmaceutically acceptable salt or a solvate thereof. An example of such proliferative diseases or disorders includes, but is not limited to, cancer.

According to another aspect of the present invention there is provided a method for inhibiting vascular endothelial growth factor (VEGF), comprising contacting VEGF with an effective amount of a compound of formula (I).

According to another aspect of the present invention there is provided a method for the treatment of angiogenesis related diseases or disorders in a subject, comprising administering to the subject a therapeutically effective amount of a compound of formula (I) or a stereoisomer or a tautomer, or an N-oxide or a pharmaceutically acceptable salt or a solvate thereof.

According to another aspect of the present invention, there is provided a method for the treatment of diseases or disorders mediated by VEGF in a subject, comprising administering to the subject a therapeutically effective amount of a compound of formula (I) or a stereoisomer, a tautomer, a polymorph, a prodrug, an N-oxide, a pharmaceutically acceptable salt and a solvate thereof.

According to another aspect of the present invention there is provided a method for the treatment of angiogenesis related diseases or disorders mediated by PI3K, mTOR, ALK-1, ALK-2 or VEGF in a subject, comprising administering to the subject a therapeutically effective amount of a compound of formula (I) or a stereoisomer, a tautomer, a polymorph, a prodrug, an N-oxide, a pharmaceutically acceptable salt and a solvate thereof.

According to another aspect of the present invention there is provided a method for inhibiting tumor necrosis factor-α (TNF-α) or interleukin-6 (IL-6), comprising contacting TNF-α or IL-6 with an effective amount of a compound of formula (I).

According to another aspect of the present invention there is provided a method for the treatment of inflammatory diseases or disorders in a subject, comprising administering to the subject a therapeutically effective amount of a compound of formula (I) or a stereoisomer or a tautomer, or an N-oxide or a pharmaceutically acceptable salt or a solvate thereof.

According to another aspect of the present invention, there is provided a method for the treatment of diseases or disorders mediated by TNF-α or IL-6 in a subject, comprising administering to the subject a therapeutically effective amount of a compound of formula (I) or a stereoisomer, a tautomer, a polymorph, a prodrug, an N-oxide, a pharmaceutically acceptable salt and a solvate thereof.

According to yet another aspect of the present invention there is provided a compound of formula (I) or a pharmaceutically acceptable salt thereof for use in the treatment of proliferative diseases or disorders mediated by PI3K, mTOR, ALK-1 or ALK-2. An example of such proliferative diseases or disorders includes, but is not limited to, cancer.

According to yet another aspect of the present invention there is provided a compound of formula (I) or a pharmaceutically acceptable salt thereof for use in the treatment of angiogenesis related diseases or disorders mediated by PI3K, mTOR, ALK-1, ALK-2 or VEGF.

According to yet another aspect of the present invention there is provided a compound of formula (I) or a pharmaceutically acceptable salt thereof for use in the treatment of diseases mediated by TNF-α or IL-6.

According to yet another aspect of the present invention there is provided a compound of formula (I) or a pharmaceutically acceptable salt thereof for use in the treatment of inflammatory diseases or disorders.

According to another aspect of the present invention there is provided a pharmaceutical composition, comprising a compound of formula (I) or a pharmaceutically acceptable salt thereof in association with a pharmaceutically acceptable carrier, adjuvant, or vehicle.

These and other objectives and advantages of the present invention will be apparent to those skilled in the art from the following description.

### BRIEF DESCRIPTION OF THE DRAWINGS

FIGS. 1A-1E are reproductions of Western blots showing the effect of certain compounds of the invention on the key proteins of the PI3K/mTOR pathway.

FIG. 2 is a scan of endothelial cells showing the effect of the Example 3a on VEGF (40 ng/mL) induced tube formation.

FIG. 3A is a graph of Tumor Weight versus Days post tumor transplantation for mice with human PC3 xenograft tumors administered with the compound of Example 19, or with the compound of Example 3a at the indicated dose and route.

FIG. 3B is a graph of Tumor Weight versus Days post tumor transplantation for mice with human PANC-1 xenograft tumors administered with the compound of Example 3a.

FIG. 4A is a graph of the change in paw thickness versus day of study for arthritic DBA/1 J mice treated with the compound of Example 19, Enbrel and vehicle (0.5% CMC).

FIG. 4B is a graph of the change in articular index versus day of study for arthritic DBA/1 J mice treated with the compound of Example 19, Enbrel and vehicle (0.5% CMC).

### DETAILED DESCRIPTION OF THE INVENTION

### Definitions

Listed below are definitions, which apply to the terms as they are used throughout the specification and the appended claims (unless they are otherwise limited in specific instances), either individually or as part of a larger group. It will be understood that “substitution” or “substituted by” or “substituted with” includes the implicit proviso that such substitution is in accordance with the permitted valence of the substituted atom and the substituent, as well as represents a stable compound, which does not readily undergo transformation such as by rearrangement, cyclization, elimination, etc.

The term “halo” or “halogen” as used herein refers to an atom selected from F, Cl, Br and I.

The term “alkyl” whether used alone or as part of a substituent group, refers to the radical of saturated aliphatic groups, including straight or branched-chain containing from 1 to 12 carbon atoms, for example, 1 to 6 carbons atoms, such as 1 to 4 carbon atoms. Examples of alkyl groups include but are not limited to methyl, ethyl, propyl, butyl, isopropyl, isobutyl, 1-methylbutyl, sec-butyl, tert-butyl, pentyl, neo-pentyl, n-hexyl, n-decyl, tetradecyl and the like.

The term “alkenyl” refers to an unsaturated, branched or straight chain alkyl group having from 2 to 10 carbon atoms, suitably 2 to 4 carbon atoms and at least one carbon-carbon double bond (two adjacent sp² carbon atoms). Depending on the placement of double bond and substituents if any, the geometry of the double bond may be entgegen (E), or zusammen (Z), cis or trans. Examples of alkenyl include but are not limited to ethenyl(vinyl), 1-propenyl(allyl), 2-propenyl and the like.

As used herein the term “haloalkyl”, means alkyl radical which is substituted by one or more halogen atoms (F, Cl, Br or I). An example of a haloalkyl is a halo (C₁-C₄)alkyl including, but not limited to, fluoromethyl, difluoromethyl, trifluoromethyl, 2-fluoroethyl, 2,2-difluoroethyl, 2,2,2-trifluoroethyl, 2,2,2-trifluoro-1,1-dimethylethyl, 2,2,2-trichloroethyl, 3-fluoropropyl, 4-fluorobutyl, chloromethyl, trichloromethyl, iodomethyl, bromomethyl and 4,4,4-trifluoro-3-methylbutyl groups. Preferred halo(C₁-C₄)alkyl groups are fluoromethyl, difluoromethyl, trifluoromethyl, 2-fluoroethyl, 2,2-difluoroethyl, 2,2,2-trifluoroethyl and 2,2,2-trifluoro-1,1-dimethylethyl groups.

The term “aryl” as used herein refers to a monocyclic or polycyclic hydrocarbon group having 6 to 14 ring carbon atoms, preferably up to 10 ring carbon atoms, more preferably up to 6 ring carbon atoms in which at least one carbocyclic ring is present that has a conjugated π electron system. Accordingly, the term “aryl” refers to C₆-C₁₄ aryl. Examples of aryl include but are not limited to phenyl, naphthyl, tetrahydronaphthyl and the like. Aryl residues can be bonded via any desired position, and in substituted aryl residues, the substituents can be located in any desired position.

In some embodiments, a C₆-C₁₄ aryl is selected from the group consisting of phenyl, indenyl, naphthyl, azulenyl, heptalenyl, biphenyl, indacenyl, acenaphthylenyl, fluorenyl, 1H-phenalenyl, phenanthrenyl or anthracenyl. In some embodiments, —C₆-C₁₄ aryl is selected from the group consisting of phenyl, naphthyl, anthracenyl and 1H-phenalenyl.

The term “heteroaryl” as used herein refers to an aromatic heterocyclic ring system containing 5 to 20 ring atoms, suitably 5 to 10 ring atoms, which may be a monocyclic or polycyclic, fused together or linked covalently. The rings may contain from 1 to 4 heteroatoms selected from N, O and S, wherein the N or S atom is optionally oxidized, or the N atom is optionally quaternized. Any suitable ring position of the heteroaryl moiety may be covalently linked to the defined chemical structure. Examples of heteroaryl include, but are not limited to, furanyl, thiophenyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzothiazolyl, benzofuranyl, benzothienyl, phthalazinyl, dibenzofuranyl, benzimidazolyl, indolyl, isoindolyl, indazolyl, quinolinyl, isoquinolinyl, quinazolinyl, quinoxalinyl, purinyl, indolizinyl, benzoisothiazolyl, benzoxazolyl, pyrrolopyridyl, furopyridinyl, benzothiadiazolyl, benzooxadiazolyl, benzotriazolyl, benzodiazolyl, dibenzothienyl and the like.

The foregoing heteroaryl groups may be C-attached or N-attached (where such an attachment is possible). For instance, a group derived from pyrrole may be pyrrol-1-yl (N-attached) or pyrrol-3-yl (C-attached).

The term “heterocyclyl” or “heterocycle” as used herein refers to a saturated or partially unsaturated monocyclic or polycyclic ring system containing 5 to 20 ring atoms of which 1, 2, 3 or 4 are identical or different heteroatoms selected from N, O and S. The “heterocyclyl” or “heterocycle” may, for example, have 1 to 2 oxygen atoms and/or 1 to 2 sulfur atoms and/or 1 to 4 nitrogen atoms in the ring. The “heterocyclyl” or “heterocycle” preferably is a 5- or 6-membered ring. The ring heteroatoms can be present in any position with respect to each other provided that the resulting “heterocyclyl” or “heterocycle” is stable. Examples of “heterocyclyl” or “heterocycle” include but are not limited to: decahydroquinolinyl, oxadiazolidinyl, imidazolidinyl, indolinyl, isobenzofuranyl, morpholinyl, octahydroisoquinolinyl, oxazolidinyl, piperidinyl, piperazinyl, pyrazolinyl, pyrazolidinyl, pyrrolidinyl, pyrrolinyl, tetrahydrofuranyl, benzodioxolyl, tetrahydroisoquinolinyl, and tetrahydroquinolinyl.

The term “alkylheterocyclyl” as used herein refers to a heterocyclyl group bonded through an alkyl, wherein the terms “alkyl” and “heterocycle” are as defined herein above. Examples of alkylheterocycle include but are not limited to piperazin-1-ylmethyl, piperidin-1-ylmethyl, pyrrolidin-2-ylmethyl, 2-morpholinoethyl and the like.

The term “alkylheteroaryl” as used herein refers to a heteroaryl group bonded through an alkyl, wherein the terms “alkyl” and “heteroaryl” are as defined herein above. Examples of alkylheteroaryl include but are not limited to pyrazolylmethyl, pyrazolylethyl, pyridylmethyl, pyridylethyl, thiazolylmethyl, thiazolylethyl, imidazolylmethyl, imidazolylethyl, thienylmethyl, thienylethyl, furanylmethyl, furanylethyl, isoxazolylmethyl, isoxazolylethyl, pyrazinylmethyl and pyrazinylethyl and the like.

The term “compound of the present invention” and “compound of this invention” and “compounds of formula (I)” includes compounds of formula (I) and stereoisomers, tautomers, N-oxides, solvates, polymorphs, prodrugs; pharmaceutically acceptable salts or solvates thereof.

The term “stereoisomer” as used herein refers to all isomers of individual compounds that differ only in the orientation of their atoms in space. The term stereoisomer includes mirror image isomers (enantiomers), mixtures of mirror image isomers (racemates, racemic mixtures), geometric (cis/trans or syn/anti or E/Z) isomers, and isomers of compounds with more than one chiral center that are not mirror images of one another (diastereoisomers). The compounds of the present invention may have asymmetric centers and occur as racemates, racemic mixtures, individual diastereoisomers, or enantiomers, or may exist as geometric isomers, with all isomeric forms of said compounds being included in the present invention.

The term “tautomer” as used herein refers to the coexistence of two (or more) compounds that differ from each other only in the position of one (or more) mobile atoms and in electron distribution, for example, keto-enol and imine-enamine tautomers.

The term “solvate” as used herein refers to a compound formed by the interaction of a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Examples for suitable solvates are the mono- or dihydrates or alcoholates of the compounds according to the invention.

The term “pharmaceutically acceptable salts” as used herein refers to inorganic and organic salts of a compound of the invention. The compounds of the present invention represented by formula (I), which contain acidic groups, may be converted into salts with pharmaceutically acceptable bases. Such salts include, for example, alkali metal salts, like lithium, sodium and potassium salts; alkaline earth metal salts like calcium and magnesium salts; ammonium salts; [tris(hydroxymethyl)aminomethane], trimethylamine salts and diethylamine salts; salts with amino acids such as lysine, arginine, guanidine and the like.

The compounds of the present invention represented by formula (I), which contain one or more basic groups, i.e. groups which can be protonated, can form an addition salt with an inorganic or organic acid. Examples of suitable acid addition salts include: hydrochlorides, hydrobromides, hydrofluorides, nitrates, acetates, alginates, ascorbates, aspartates, benzoates, benzenesulfonates, bisulfates, borates, cinnamates, citrates, ethanesulfonates, fumarates, glucuronates, glutamates, glycolates, ketoglutarates, lactates, maleates, malonates, mesylates, oxalates, palmoates, perchlorates, phosphates, picrates, salicylates, succinates, sulfamates, sulfates, tartrates, tosylates and other acids known to the person skilled in the art.

The term “N-oxide” as used herein in reference to the compounds of formula (I) refers to the oxide of the nitrogen atom of a nitrogen-containing heteroaryl or heterocycle. N-oxide can be formed in presence of an oxidizing agent for example peroxide such as m-chloro-perbenzoic acid or hydrogen peroxide.

Various polymorphs of compounds of formula (I), forming part of this invention may be prepared by crystallization of compounds of formula (I) under different conditions. The different conditions are, for example, using different commonly used solvents or their mixtures for crystallization; crystallization at different temperatures; various modes of cooling, ranging from very fast to very slow cooling during crystallizations. Polymorphs may also be obtained by heating or melting the compound followed by gradual or fast cooling. The presence of polymorphs may be determined by infrared spectroscopy, solid probe nuclear magnetic resonance (NMR) spectroscopy, differential scanning calorimetry, powder x-ray diffraction or such other techniques.

The term “prodrug” as used herein refers to a compound that is a drug precursor, which, following administration into or onto the body, releases the drug in vivo via a chemical or physiological process, e.g., a prodrug on being brought to physiological pH or through an enzyme action is converted to the desired drug form. Various forms of prodrugs are known in the art and further information is discussed in Pro-drugs as Novel Delivery Systems, Vol. 14, ACS Symposium Series (T. Higuchi and W. Stella), Bioreversible Carriers in Drug Design, Pergamon Press, 1987 (Ed. E. B. Roche, American Pharmaceutical Association) and Design of Prodrugs, Elsevier 1985, (edited by H. Bundgaard). Exemplary prodrugs include esters of carboxylic acids such as methyl and ethyl esters, ethers of alcohols and amides of amines Pharmaceutically acceptable esters can be converted under physiological conditions to the carboxylic acid of formula (I).

The present invention also includes within its scope all isotopically labeled forms of compounds of formula (I), wherein one or more atoms of compounds of formula (I) are replaced by their respective isotopes. All isotopes of any particular atom or element as specified are contemplated within the scope of the compounds of the invention. Examples of isotopes that may be incorporated into the compounds disclosed herein include, but are not limited to, isotopes of hydrogen such as ²H and ³H, carbon such as ¹¹C, ¹³C and ¹⁴C, nitrogen such as ¹³N and ¹⁵N, oxygen such as ¹⁵O, ¹⁷P and ¹⁸O, chlorine such as ³⁶Cl, fluorine such as ¹⁸F and sulphur such as ³⁵S. Substitution with heavier isotopes, for example, replacing one or more key carbon-hydrogen bonds with carbon-deuterium bond may show certain therapeutic advantages, resulting from longer metabolism cycles, (e.g., increased in-vivo half life or reduced dosage requirements), improved safety or greater effectiveness and hence may be preferred in certain circumstances.

### Embodiments

The present invention provides compounds of formula (I),

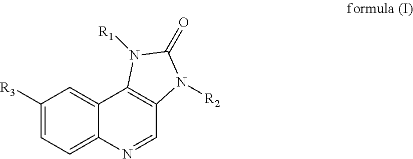

or stereoisomers, tautomers, polymorphs, prodrugs, N-oxides, pharmaceutically acceptable salts or solvates thereof, wherein,

R₁ is selected from alkylheterocyclyl, alkylheteroaryl or heteroaryl, wherein each of heterocyclyl and heteroaryl is optionally substituted with one or more groups selected from R₁₁;

R₂ is —C₁-C₄ alkyl, optionally substituted with one or more groups independently selected from

—CN or —C₂-C₄ alkenyl;

R₃ is selected from heteroaryl or —C₆-C₁₄ aryl, wherein each of aryl and heteroaryl is optionally substituted with one or more groups selected from R₃₁;

R₁₁ at each occurrence is independently selected from halogen, —CN, —ORₓ, —NRₓR_{y}, —NRₓCOR_{y}, —COORₓ, —CONRₓR_{y}, halo-C₁-C₄ alkyl, —C₁-C₄ alkyl, heterocyclyl or heteroaryl, wherein each of alkyl, heterocyclyl, and heteroaryl is optionally substituted with one or more groups independently selected from CN or —C₁-C₄ alkyl;

R₃₁ at each occurrence is independently selected from halogen, —ORₓ, —CN, —NRₓR_{y}, —NRₓCOR_{y}, —COORₓ, —CONRₓR_{y}, halo-C₁-C₄ alkyl or —C₁-C₄ alkyl;

wherein Rₓ and R_{y} at each occurrence are independently selected from hydrogen or —C₁-C₄ alkyl.

One embodiment of the present invention is a compound of formula (I), wherein R₁ is heteroaryl, wherein heteroaryl is optionally substituted with one or more groups selected from R₁₁.

Another embodiment is a compound of formula (I), wherein R₁ is a heteroaryl, wherein the heteroaryl is optionally substituted with one or more groups independently selected from halogen, —CN, —ORₓ, —NRₓR_{y}, halo-C₁-C₄ alkyl, —C₁-C₄ alkyl, heterocyclyl or heteroaryl, wherein each of —C₁-C₄ alkyl, heterocyclyl or heteroaryl is optionally substituted with one or more groups independently selected from CN or —C₁-C₄ alkyl and Rₓ and R_{y} at each occurrence are independently selected from hydrogen or —C₁-C₄ alkyl.

Another embodiment is a compound of formula (I), wherein R₁ is a heteroaryl selected from pyridyl, pyrimidinyl or quinolinyl, wherein the pyridyl, pyrimidinyl and quinolinyl are optionally substituted with one or more groups selected from R₁₁.

Another embodiment is a compound of formula (I), wherein R₁ is a heteroaryl selected from pyridyl, pyrimidinyl or quinolinyl, wherein the pyridyl, pyrimidinyl and quinolinyl are optionally substituted with one or more groups independently selected from halogen, —CN, —ORₓ, —NRₓR_{y}, halo-C₁-C₄ alkyl, —C₁-C₄ alkyl, heterocyclyl or heteroaryl, wherein each of the —C₁-C₄ alkyl, heterocyclyl and heteroaryl is optionally substituted with one or more groups independently selected from —CN or —C₁-C₄ alkyl and Rₓ and R_{y} at each occurrence are independently selected from hydrogen or —C₁-C₄ alkyl.

Another embodiment is a compound of formula (I), wherein R₁ is pyridyl, optionally substituted with one or more groups selected from R₁₁.

Another embodiment is a compound of formula (I), wherein R₁ is pyridyl, optionally substituted with one or more groups independently selected from halogen, —CN, —ORₓ, —NRₓR_{y}, halo-C₁-C₄ alkyl, —C₁-C₄ alkyl, heterocyclyl or heteroaryl, wherein each of —C₁-C₄ alkyl, heterocyclyl and heteroaryl is optionally substituted with one or more groups independently selected from CN or —C₁-C₄ alkyl and Rₓ and R_{y} at each occurrence are independently selected from hydrogen or —C₁-C₄ alkyl.

Another embodiment is a compound of formula (I), wherein R₁ is 3-pyridyl, optionally substituted with one or more groups selected from R₁₁.

Another embodiment is a compound of formula (I), wherein R₁ is represented by the structural formula

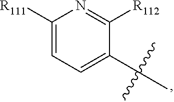

wherein each of R₁₁₁ and R₁₁₂ is independently selected from hydrogen, halogen, —CN, —ORₓ, —NRₓR_{y}, halo-C₁-C₄ alkyl, —C₁-C₄ alkyl, heterocyclyl or heteroaryl, wherein the —C₁-C₄ alkyl is optionally substituted with —CN and the heterocyclyl and heteroaryl are optionally substituted with —C₁-C₄ alkyl; Rₓ and R_{y} at each occurrence are independently selected from hydrogen or —C₁-C₄ alkyl and the symbol [symbol] indicates the point of attachment to the rest of the molecule.

Another embodiment is a compound of formula (I), wherein R₁ is represented by the structural formula

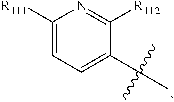

wherein each of R₁₁₁ and R₁₁₂ is independently selected from hydrogen, halogen, —CN, —OCH₃, —N(CH₃)₂, —CF₃, —C₁-C₄ alkyl, morpholinyl, piperazinyl or pyridyl, wherein the —C₁-C₄ alkyl is optionally substituted with —CN, and the piperazinyl is optionally substituted with —C₁-C₄ alkyl and the symbol [symbol] indicates the point of attachment to the rest of the molecule.

Another embodiment is a compound of formula (I), wherein R₁ is represented by the structural formula

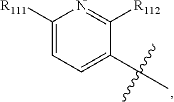

wherein R₁₁₁ is selected from —Cl, —CN, —OCH₃, —OC₂H₅, —N(CH₃)₂, —CF₃, —C(CH₃)₂CN, morpholinyl or piperazinylmethyl; R₁₁₂ is selected from hydrogen, Cl, CH₃ or pyridyl and the symbol [symbol] indicates the point of attachment to the rest of the molecule.

Another embodiment is a compound of formula (I), wherein R₁ is quinolinyl, optionally substituted with one or more groups selected from R₁₁.

Another embodiment is a compound of formula (I), wherein R₁ is quinolinyl.

Another embodiment is a compound of formula (I), wherein R₁ is pyrimidinyl, optionally substituted with one or more groups selected from R₁₁.

Another embodiment is a compound of formula (I), wherein R₁ is pyrimidinyl, optionally substituted with halo-C₁-C₄-alkyl.

Another embodiment is a compound of formula (I), wherein R₁ is alkylheterocyclyl, wherein the heterocyclyl moiety is optionally substituted with one or more groups selected from R₁₁.

Another embodiment is a compound of formula (I), wherein R₁ is 2-morpholinoethyl.

Another embodiment is a compound of formula (I), wherein R₁₁ is —C₁-C₄ alkyl, optionally substituted with —CN.

Another embodiment is a compound of formula (I), wherein R₁₁ is halo-C₁-C₄ alkyl.

Another embodiment is a compound of formula (I), wherein R₁₁ is —ORₓ, wherein Rₓ is selected from hydrogen or —C₁-C₄ alkyl.

Another embodiment is a compound of formula (I), wherein R₁₁ is heterocyclyl, optionally substituted with —C₁-C₄ alkyl.

Another embodiment is a compound of formula (I), wherein R₁₁ is heteroaryl.

Another embodiment is a compound of formula (I), wherein R₁₁ is pyridyl.

Another embodiment is a compound of formula (I), wherein R₂ is methyl optionally substituted with one or more groups independently selected from —CN or —C₂-C₄ alkenyl.

Another embodiment is a compound of formula (I), wherein R₂ is methyl.

Another embodiment is a compound of formula (I), wherein R₂ is cyanomethyl.

Another embodiment is a compound of formula (I), wherein R₂ is allyl.

Another embodiment is a compound of formula (I), wherein R₃ is heteroaryl optionally substituted with one or more groups selected from R₃₁.

Another embodiment is a compound of formula (I), wherein R₃ is heteroaryl optionally substituted with one or more groups independently selected from halogen, —ORₓ, —NRₓR_{y}, —C₁-C₄-alkyl or halo-C₁-C₄-alkyl, wherein Rₓ and R_{y} at each occurrence are independently selected from hydrogen or —C₁-C₄ alkyl.

Another embodiment is a compound of formula (I), wherein R₃ is selected from pyridyl or quinolinyl optionally substituted with one or more groups selected from R₃₁.

Another embodiment is a compound of formula (I), wherein R₃ is selected from pyridyl or quinolinyl optionally substituted with one or more groups independently selected from halogen, —ORₓ, NRₓR_{y}, —C₁-C₄-alkyl or halo-C₁-C₄-alkyl, wherein Rₓ and R_{y} at each occurrence are independently selected from hydrogen and —C₁-C₄ alkyl.

Another embodiment is a compound of formula (I), wherein R₃ is pyridyl optionally substituted with one or more groups selected from R₃₁.

Another embodiment is a compound of formula (I), wherein R₃ is pyridyl optionally substituted with one or more groups independently selected from halogen, —ORₓ, NRₓR_{y}, —C₁-C₄-alkyl or halo-C₁-C₄-alkyl, wherein Rₓ and R_{y} at each occurrence are independently selected from hydrogen or —C₁-C₄ alkyl.

Another embodiment is a compound of formula (I), wherein R₃ is 3-pyridyl optionally substituted with one or more groups selected from R₃₁.

Another embodiment is a compound of formula (I), wherein R₃ is represented by the structural formula

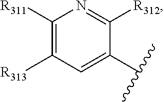

wherein each of R₃₁₁, R₃₁₂ and R₃₁₃ is independently selected from hydrogen, halogen, —ORₓ, —NRₓR_{y}, —C₁-C₄-alkyl or halo-C₁-C₄-alkyl, wherein Rₓ and R_{y} at each occurrence are independently selected from hydrogen or —C₁-C₄ alkyl.

Another embodiment is a compound of formula (I), wherein R₃ is represented by the structural formula

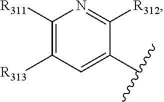

wherein each of R₃₁₁, R₃₁₂ and R₃₁₃ is independently selected from hydrogen, halogen, —O—C₁-C₄ alkyl, —NH₂, —NH—C₁-C₄-alkyl, —N(C₁-C₄-alkyl)₂ or methyl, wherein methyl is optionally substituted with one to three halogen atoms and the symbol [symbol] indicates the point of attachment to the rest of the molecule.

Another embodiment is a compound of formula (I), wherein R₃ is represented by the structural formula

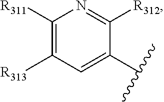

wherein each of R₃₁₁, R₃₁₂ and R₃₁₃ is independently selected from hydrogen, F, —OCH₃, —NH₂, —NH—CH₃, —N(CH₃)₂ or —CF₃ and the symbol [symbol] indicates the point of attachment to the rest of the molecule.

Another embodiment is a compound of formula (I), wherein R₃ is represented by the structural formula

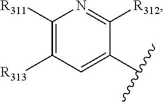

wherein R₃₁₁ is —NH₂; R₃₁₂ and R₃₁₃ are independently selected from hydrogen, halogen, —O—C₁-C₄ alkyl, —NH₂, —NH—C₁-C₄-alkyl, —N(C₁-C₄-alkyl)₂ or methyl, wherein methyl is optionally substituted with one to three halogen atoms and the symbol [symbol] indicates the point of attachment to the rest of the molecule.

Another embodiment is a compound of formula (I), wherein R₃ is represented by the structural formula

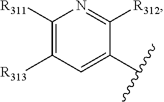

wherein R₃₁₃ is —CF₃ and R₃₁₁ and R₃₁₂ are independently selected from hydrogen, halogen, —O—C₁-C₄ alkyl, —NH₂, —NH—C₁-C₄-alkyl, —N(C₁-C₄-alkyl)₂ or methyl, wherein methyl is optionally substituted with one to three halogen atoms and the symbol [symbol] indicates the point of attachment to the rest of the molecule.

Another embodiment is a compound of formula (I), wherein R₃ is represented by the structural formula

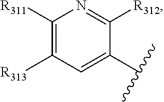

wherein R₃₁₁ is —NH₂ and R₃₁₃ is —CF₃ and R₃₁₂ is selected from hydrogen, halogen, —O—C₁-C₄ alkyl, —NH₂, —NH—C₁-C₄-alkyl, —N(C₁-C₄-alkyl)₂ or methyl; wherein methyl is optionally substituted with one to three halogen atoms and the symbol [symbol] indicates the point of attachment to the rest of the molecule.

Another embodiment is a compound of formula (I), wherein R₃ is represented by the structural formula

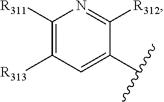

wherein R₃₁₁ is —NH₂, R₃₁₃ is —CF₃ and R₃₁₂ is hydrogen and the symbol [symbol] indicates the point of attachment to the rest of the molecule.

Another embodiment is a compound of formula (I), wherein R₃ is quinolinyl optionally substituted with —OR, wherein R is selected from hydrogen or —C₁-C₄ alkyl.

Another embodiment is a compound of formula (I), wherein R₃ is quinolinyl.

Another embodiment is a compound of formula (I), wherein R₃ is quinolinyl substituted with OH.

Another embodiment is a compound of formula (I), wherein R₃ is pyrimidinyl optionally substituted with —ORₓ, wherein Rₓ is selected from hydrogen or —C₁-C₄ alkyl.

Another embodiment is a compound of formula (I), wherein R₃ is pyrimidinyl.

Another embodiment is a compound of formula (I), wherein R₃ is pyrimidinyl optionally substituted with —OCH₃.

Another embodiment is a compound of formula (I), wherein R₃ is —C₆-C₁₄ aryl optionally substituted with one or more groups selected from R₃₁.

Another embodiment is a compound of formula (I), wherein R₃ is —C₆-C₁₄ aryl optionally substituted with one or more groups independently selected from halogen, —ORₓ or methyl, wherein methyl is optionally substituted with one to three halogen atoms, wherein Rₓ is selected from hydrogen or —C₁-C₄ alkyl.

Another embodiment is a compound of formula (I), wherein R₃ is phenyl optionally substituted with one or more groups independently selected from halogen, —ORₓ or methyl, wherein methyl is optionally substituted with one to three halogen atoms, wherein Rₓ is selected from hydrogen and —C₁-C₄ alkyl.

Another embodiment is a compound of formula (I), wherein R₃ is phenyl optionally substituted with one or more groups independently selected from F, —OCH₃ or CF₃.

Another embodiment is a compound of formula (I), wherein R₁ is heteroaryl optionally substituted with one or more groups selected from R₁₁; R₂ is —C₁-C₄ alkyl optionally substituted with one of more groups independently selected from CN or —C₂-C₄ alkenyl; and R₃ is heteroaryl or —C₆-C₁₄ aryl optionally substituted with one or more groups selected from R₃₁.

Another embodiment is a compound of formula (I), wherein R₁ is heteroaryl optionally substituted with one or more groups selected from R₁₁; R₂ is —C₁-C₄ alkyl optionally substituted with one or more groups independently selected from —CN or —C₂-C₄ alkenyl; and R₃ is heteroaryl optionally substituted with one or more groups selected from R₃₁.

Another embodiment is a compound of formula (I), wherein R₁ is heteroaryl optionally substituted with one or more groups selected from halogen, —CN, —ORₓ, —NRₓR_{y}, halo-C₁-C₄ alkyl, —C₁-C₄ alkyl, heterocyclyl or heteroaryl, wherein each of —C₁-C₄ alkyl and heterocyclyl is optionally substituted with one or more groups independently selected from CN or —C₁-C₄ alkyl; R₂ is —C₁-C₄ alkyl optionally substituted with one or more groups independently selected from —CN or —C₂-C₄ alkenyl; and R₃ is heteroaryl optionally substituted with one or more groups independently selected from halogen, —ORₓ, —NRₓR_{y} or halo-C₁-C₄ alkyl, wherein Rₓ and R_{y} at each occurrence are independently selected from hydrogen or —C₁-C₄ alkyl.

Another embodiment is a compound of formula (I), wherein R₁ is heteroaryl optionally substituted with one or more groups independently selected from Cl, —CN, —OCH₃, —OC₂H₅, —N(CH₃)₂, —CF₃, —CH₃, —C(CH₃)₂CN, morpholinyl, piperazinyl or pyridyl; R₂ is methyl optionally substituted with —CN; and R₃ is heteroaryl optionally substituted with one or more groups independently selected from F, —OH, —OCH₃, —NH₂, —NHCH₃, —N(CH₃)₂ or —CF₃.

Another embodiment is a compound of formula (I), wherein R₁ is selected from pyridyl, pyrimidinyl or quinolinyl, wherein pyridyl, pyrimidinyl and quinolinyl are optionally substituted with one or more groups selected from R₁₁; R₂ is methyl optionally substituted with one or more groups independently selected from CN or —C₂-C₄ alkenyl; and R₃ is selected from pyridyl or quinolinyl, wherein pyridyl and quinolinyl are optionally substituted with one or more groups selected from R₃₁.

Another embodiment is a compound of formula (I), wherein R₁ is selected from pyridyl, pyrimidinyl or quinolinyl, wherein pyridyl, pyrimidinyl and quinolinyl are optionally substituted with one or more groups independently selected from halogen, —CN, —ORₓ, —NRₓR_{y}, halo-C₁-C₄ alkyl, —C₁-C₄ alkyl, heterocyclyl or heteroaryl, wherein each of —C₁-C₄ alkyl, heterocyclyl and heteroaryl is optionally substituted with one or more groups independently selected from —CN or —C₁-C₄ alkyl; R₂ is allyl or methyl optionally substituted with —CN; and R₃ is selected from pyridyl or quinolinyl; wherein pyridyl and quinolinyl are optionally substituted with one or more groups independently selected from halogen, —ORₓ, NRₓR_{y}, —C₁-C₄-alkyl or halo-C₁-C₄-alkyl, wherein Rₓ and R_{y} at each occurrence are independently selected from hydrogen or —C₁-C₄ alkyl.

Another embodiment is a compound of formula (I), wherein R₁ is heteroaryl optionally substituted with one or more groups selected from R₁₁; R₂ is allyl or —C₁-C₄ alkyl, wherein —C₁-C₄ alkyl is optionally substituted with CN; and R₃ is —C₆-C₁₄ aryl optionally substituted with one or more groups selected from R₃₁.

Another embodiment is a compound of formula (I), wherein the pharmaceutically acceptable salt of the compound of formula (I) is selected from (a) an inorganic acid addition salt selected from hydrochloride, sulphate, phosphate or nitrate, and (b) an organic acid addition salt selected from acetate, maleate, tartarate, citrate, mesylate, tosylate or cinnamate.

Representative compounds, encompassed in accordance with the present invention include:
2-Methyl-2-(5-(3-methyl-2-oxo-8-(pyridin-3-yl)-2,3-dihydro-1H-imidazo[4,5-c]quinolin-1-yl)pyridin-2-yl)propanenitrile,
2-Methyl-2-(5-(3-methyl-2-oxo-8-(quinolin-3-yl)-2,3-dihydro-1H-imidazo[4,5-c]quinolin-1-yl)pyridin-2-yl)propanenitrile,
2-(5-(8-(6-Amino-5-(trifluoromethyl)pyridin-3-yl)-3-methyl-2-oxo-2,3-dihydro-1H-imidazo[4,5-c]quinolin-1-yl)pyridin-2-yl)-2-methylpropanenitrile,
2-Methyl-2-(5-(3-methyl-2-oxo-8-(5-(trifluoromethyl)pyridin-3-yl)-2,3-dihydro-1H-imidazo[4,5-c]quinolin-1-yl)pyridin-2-yl)propanenitrile,
2-Methyl-2-(5-(3-methyl-2-oxo-8-(quinolin-6-yl)-2,3-dihydro-1H-imidazo[4,5-c]quinolin-1-yl)pyridin-2-yl)propanenitrile,
2-(5-(8-(Isoquinolin-4-yl)-3-methyl-2-oxo-2,3-dihydro-1H-imidazo[4,5-c]quinolin-1-yl)pyridin-2-yl)-2-methylpropanenitrile,
2-(5-(8-(2-Hydroxyquinolin-3-yl)-3-methyl-2-oxo-2,3-dihydro-1H-imidazo[4,5-c]quinolin-1-yl)pyridin-2-yl)-2-methylpropanenitrile,
2-(5-(8-(6-(Dimethylamino) pyridin-3-yl)-3-methyl-2-oxo-2,3-dihydro-1H-imidazo[4,5-c]quinolin-1-yl)pyridin-2-yl)-2-methylpropanenitrile,
2-Methyl-2-(5-(3-methyl-2-oxo-8-(pyrimidin-5-yl)-2,3-dihydro-1H-imidazo[4,5-c]quinolin-1-yl)pyridin-2-yl)propanenitrile,
2-(5-(8-(2,6-Difluoropyridin-3-yl)-3-methyl-2-oxo-2,3-dihydro-1H-imidazo[4,5-c]quinolin-1-yl)pyridin-2-yl)-2-methylpropanenitrile,
2-(5-(8-(5-Fluoro-2-methoxyphenyl)-3-methyl-2-oxo-2,3-dihydro-1H-imidazo[4,5-c]quinolin-1-yl)pyridin-2-yl)-2-methylpropanenitrile,
2-(5-(8-(2-Fluoro-5-(trifluoromethyl)phenyl)-3-methyl-2-oxo-2,3-dihydro-1H-imidazo[4,5-c]quinolin-1-yl)pyridin-2-yl)-2-methylpropanenitrile,
2-(5-(8-(2,4-Dimethoxypyrimidin-5-yl)-3-methyl-2-oxo-2,3-dihydro-1H-imidazo[4,5-c]quinolin-1-yl)pyridin-2-yl)-2-methylpropanenitrile,
2-(5-(3-(Cyanomethyl)-2-oxo-8-(pyridin-3-yl)-2,3-dihydro-1H-imidazo[4,5-c]quinolin-1-yl)pyridin-2-yl)-2-methylpropanenitrile,
1-(6-(Dimethylamino)pyridin-3-yl)-3-methyl-8-(pyridin-3-yl)-1H-imidazo[4,5-c]quinolin-2(3H)-one,
2-(5-(8-(6-Amino-5-(trifluoromethyl)pyridin-3-yl)-3-(cyanomethyl)-2-oxo-2,3-dihydro-1H-imidazo[4,5-c]quinolin-1-yl)pyridin-2-yl)-2-methylpropanenitrile,
2-(5-(3-(Cyanomethyl)-2-oxo-8-(quinolin-3-yl)-2,3-dihydro-1H-imidazo[4,5-c]quinolin-1-yl)pyridin-2-yl)-2-methylpropanenitrile,
2-(5-(3-Allyl-2-oxo-8-(pyridin-3-yl)-2,3-dihydro-1H-imidazo[4,5-c]quinolin-1-yl)pyridin-2-yl)-2-methylpropanenitrile,
8-(6-Amino-5-(trifluoromethyl)pyridin-3-yl)-1-(6-methoxypyridin-3-yl)-3-methyl-1H-imidazo[4,5-c]quinolin-2(3H)-one,
1-(6-Methoxypyridin-3-yl)-3-methyl-8-(quinolin-3-yl)-1H-imidazo[4,5-c]quinolin-2(3H)-one,
2-(1-(6-Methoxypyridin-3-yl)-2-oxo-8-(pyridin-3-yl)-1H-imidazo[4,5-c]quinolin-3(2H)-yl)acetonitrile,
1-(6-Methoxypyridin-3-yl)-3-methyl-8-(5-(trifluoromethyl)pyridin-3-yl)-1H-imidazo[4,5-c]quinolin-2(3H)-one,
1-(6-Methoxypyridin-3-yl)-3-methyl-8-(pyridin-3-yl)-1H-imidazo[4,5-c]quinolin-2(3H)-one,
2-(1-(6-Methoxypyridin-3-yl)-2-oxo-8-(quinolin-3-yl)-1H-imidazo[4,5-c]quinolin-3(2H)-yl)acetonitrile,
8-(6-(Dimethylamino)pyridin-3-yl)-1-(6-methoxypyridin-3-yl)-3-methyl-1H-imidazo[4,5-c]quinolin-2(3H)-one,
1-(6-Methoxypyridin-3-yl)-3-methyl-8-(6-(methylamino)-5-(trifluoromethyl)pyridin-3-yl)-1H-imidazo[4,5-c]quinolin-2(3H)-one,
8-(2-Fluoro-5-(trifluoromethyl)phenyl)-1-(6-methoxypyridin-3-yl)-3-methyl-1H-imidazo[4,5-c]quinolin-2(3H)-one,
1-(6-Methoxypyridin-3-yl)-3-methyl-8-(pyridin-4-yl)-1H-imidazo[4,5-c]quinolin-2(3H)-one,
8-(5-Fluoro-2-methoxyphenyl)-1-(6-methoxypyridin-3-yl)-3-methyl-1H-imidazo[4,5-c]quinolin-2(3H)-one,
8-(6-Amino-5-(trifluoromethyl)pyridin-3-yl)-1-(6-ethoxypyridin-3-yl)-3-methyl-1H-imidazo[4,5-c]quinolin-2(3H)-one,
8-(6-(Dimethylamino) pyridin-3-yl)-1-(6-ethoxypyridin-3-yl)-3-methyl-1H-imidazo[4,5-c]quinolin-2(3H)-one,
1-(6-Ethoxypyridin-3-yl)-3-methyl-8-(quinolin-3-yl)-1H-imidazo[4,5-c]quinolin-2(3H)-one,
8-(2,6-Difluoropyridin-3-yl)-1-(6-ethoxypyridin-3-yl)-3-methyl-1H-imidazo[4,5-c]quinolin-2(3H)-one,
1-(6-Ethoxypyridin-3-yl)-8-(2-methoxypyrimidin-5-yl)-3-methyl-1H-imidazo[4,5-c]quinolin-2(3H)-one,
1-(6-Ethoxypyridin-3-yl)-3-methyl-8-(quinolin-6-yl)-1H-imidazo[4,5-c]quinolin-2(3H)-one,
2-(1-(6-Methoxy-2-methylpyridin-3-yl)-2-oxo-8-(quinolin-3-yl)-1H-imidazo[4,5-c]quinolin-3 (2H)-yl)acetonitrile,
2-(1-(6-Methoxy-2-methylpyridin-3-yl)-2-oxo-8-(6-(trifluoromethyl)pyridin-3-yl)-1H-imidazo[4,5-c]quinolin-3 (2H)-yl)acetonitrile,
8-(6-Amino-5-(trifluoromethyl)pyridin-3-yl)-1-(6-methoxy-2-methylpyridin-3-yl)-3-methyl-1H-imidazo[4,5-c]quinolin-2(3H)-one,
1-(6-Methoxy-2-methylpyridin-3-yl)-3-methyl-8-(5-(trifluoromethyl)pyridin-3-yl)-1H-imidazo[4,5-c]quinolin-2(3H)-one,
8-(6-(Dimethylamino) pyridin-3-yl)-1-(6-methoxy-2-methylpyridin-3-yl)-3-methyl-1H-imidazo[4,5-c]quinolin-2(3H)-one,
1-(6-Methoxy-2-methylpyridin-3-yl)-3-methyl-8-(quinolin-3-yl)-1H-imidazo[4,5-c]quinolin-2(3H)-one,
5-(3-(Cyanomethyl)-2-oxo-8-(pyridin-3-yl)-2,3-dihydro-1H-imidazo[4,5-c]quinolin-1-yl)picolinonitrile,
5-(3-(1-Cyanoethyl)-2-oxo-8-(pyridin-3-yl)-2,3-dihydro-1H-imidazo[4,5-c]quinolin-1-yl)picolinonitrile,
5-(3-Methyl-2-oxo-8-(quinolin-3-yl)-2,3-dihydro-1H-imidazo[4,5-c]quinolin-1-yl)picolinonitrile,
5-(8-(6-Amino-5-(trifluoromethyl)pyridin-3-yl)-3-methyl-2-oxo-2,3-dihydro-1H-imidazo[4,5-c]quinolin-1-yl)picolinonitrile,
5-(8-(2-Fluoropyridin-3-yl)-3-methyl-2-oxo-2,3-dihydro-1H-imidazo[4,5-c]quinolin-1-yl)picolinonitrile,
5-(8-(6-Fluoropyridin-3-yl)-3-methyl-2-oxo-2,3-dihydro-1H-imidazo[4,5-c]quinolin-1-yl)picolinonitrile,
5-(8-(6-Methoxypyridin-3-yl)-3-methyl-2-oxo-2,3-dihydro-1H-imidazo[4,5-c]quinolin-1-yl)picolinonitrile,
5-(3-Methyl-2-oxo-8-(pyridin-3-yl)-2,3-dihydro-1H-imidazo[4,5-c]quinolin-1-yl)picolinonitrile,
5-(8-(6-(Dimethylamino)pyridin-3-yl)-3-methyl-2-oxo-2,3-dihydro-1H-imidazo[4,5-c]quinolin-1-yl)picolinonitrile,
5-(3-(Cyanomethyl)-2-oxo-8-(quinolin-3-yl)-2,3-dihydro-1H-imidazo[4,5-c]quinolin-1-yl)picolinonitrile,
5-(3-(1-Cyanoethyl)-2-oxo-8-(quinolin-3-yl)-2,3-dihydro-1H-imidazo[4,5-c]quinolin-1-yl)picolinonitrile,
3-Methyl-8-(pyridin-3-yl)-1-(6-(trifluoromethyl)pyridin-3-yl)-1H-imidazo[4,5-c]quinolin-2(3H)-one,
3-Methyl-8-(quinolin-3-yl)-1-(6-(trifluoromethyl)pyridin-3-yl)-1H-imidazo[4,5-c]quinolin-2(3H)-one,
8-(6-Amino-5-(trifluoromethyl)pyridin-3-yl)-3-methyl-1-(6-(trifluoromethyl)pyridin-3-yl)-1H-imidazo[4,5-c]quinolin-2(3H)-one,
3-Methyl-1,8-bis(6-(trifluoromethyl)pyridin-3-yl)-1H-imidazo[4,5-c]quinolin-2(3H)-one,
8-(2,6-Difluoropyridin-3-yl)-3-methyl-1-(6-(trifluoromethyl)pyridin-3-yl)-1H-imidazo[4,5-c]quinolin-2(3H)-one,
6-Chloro-5-(3-methyl-2-oxo-8-(quinolin-3-yl)-2,3-dihydro-1H-imidazo[4,5-c]quinolin-1-yl)picolinonitrile,
8-(6-Amino-5-(trifluoromethyl)pyridin-3-yl)-1-(2-chloro-6-(trifluoromethyl)pyridin-3-yl)-3-methyl-1H-imidazo[4,5-c]quinolin-2(3H)-one,
1-(6-Chloropyridin-3-yl)-3-methyl-8-(pyridin-3-yl)-1H-imidazo[4,5-c]quinolin-2(3H)-one,
1-(6-Chloropyridin-3-yl)-3-methyl-8-(quinolin-3-yl)-1H-imidazo[4,5-c]quinolin-2(3H)-one,
1-(2,6-Dichloropyridin-3-yl)-3-methyl-8-(pyridin-3-yl)-1H-imidazo[4,5-c]quinolin-2(3H)-one,
1-(6-Chloro-2-(trifluoromethyl)pyridin-3-yl)-3-methyl-8-(pyridin-3-yl)-1H-imidazo[4,5-c]quinolin-2(3H)-one,
1-(6-(Dimethylamino)pyridin-3-yl)-3-methyl-8-(quinolin-3-yl)-1H-imidazo[4,5-c]quinolin-2(3H)-one,
3-Methyl-8-(quinolin-3-yl)-1-(quinolin-6-yl)-1H-imidazo[4,5-c]quinolin-2(3H)-one,
3-Methyl-1-(quinolin-6-yl)-8-(5-(trifluoromethyl)pyridin-3-yl)-1H-imidazo[4,5-c]quinolin-2(3H)-one,
8-(6-Amino-5-(trifluoromethyl)pyridin-3-yl)-3-methyl-1-(quinolin-6-yl)-1H-imidazo[4,5-c]quinolin-2(3H)-one,
3-Methyl-1-(2-morpholinoethyl)-8-(pyridin-3-yl)-1H-imidazo[4,5-c]quinolin-2(3H)-one,
8-(6-Amino-5-(trifluoromethyl)pyridin-3-yl)-3-methyl-1-(2-morpholino ethyl)-1H-imidazo[4,5-c]quinolin-2(3H)-one,
3-Methyl-1-(2-morpholino ethyl)-8-(quinolin-3-yl)-1H-imidazo[4,5-c]quinolin-2(3H)-one,
3-Methyl-1-(6-(4-methylpiperazin-1-yl)pyridin-3-yl)-8-(pyridin-3-yl)-1H-imidazo[4,5-c]quinolin-2(3H)-one,
1-(6-Chloro-2,4′-bipyridin-3-yl)-3-methyl-8-(pyridin-3-yl)-1H-imidazo[4,5-c]quinolin-2(3H)-one,
3-Methyl-1-(6-morpholinopyridin-3-yl)-8-(quinolin-3-yl)-1H-imidazo[4,5-c]quinolin-2(3H)-one,
8-(6-Amino-5-(trifluoromethyl)pyridin-3-yl)-3-methyl-1-(2-(trifluoromethyl)pyrimidin-5-yl)-1H-imidazo[4,5-c]quinolin-2(3H)-one and
8-(5-Amino-6-methoxypyridin-3-yl)-1-(6-methoxypyridin-3-yl)-3-methyl-1H-imidazo[4,5-c]quinolin-2(3H)-one or
   a pharmaceutically acceptable salt, a stereoisomer, a tautomer or N-oxide thereof.

Particular compounds encompassed in accordance with the present invention include:
8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium methanesulfonate,
8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium chloride,
8-(Isoquinolin-4-yl)-1-(6-(2-cyanopropan-2-yl)pyridin-3-yl)-3-methyl-2-oxo-2,3-dihydro-1H-imidazo[4,5-c]quinolin-5-ium methanesulfonate,
8-(Isoquinolin-4-yl)-1-(6-(2-cyanopropan-2-yl)pyridin-3-yl)-3-methyl-2-oxo-2,3-dihydro-1H-imidazo[4,5-c]quinolin-5-ium chloride,
8-(6-Ammonio-5-(trifluoromethyl)pyridin-3-yl)-1-(6-methoxypyridin-3-yl)-3-methyl-2-oxo-2,3-dihydro-1H-imidazo[4,5-c]quinolin-5-ium methanesulfonate and
8-(6-Ammonio-5-(trifluoromethyl)pyridin-3-yl)-3-methyl-2-oxo-1-(6-(trifluoromethyl)pyridin-3-yl)-2,3-dihydro-1H-imidazo[4,5-c]quinolin-5-ium methanesulfonate,
   or a stereoisomer, a tautomer or an N-oxide thereof.

### Methods of Preparation

The compounds of formula (I) can be prepared using various procedures, some of which are depicted in the scheme below. Those with skill in the art will appreciate that the specific starting compounds and reagents, such as bases, solvents, coupling agents; temperature conditions etc. identified in the Scheme can be altered to prepare compounds encompassed by the present invention.

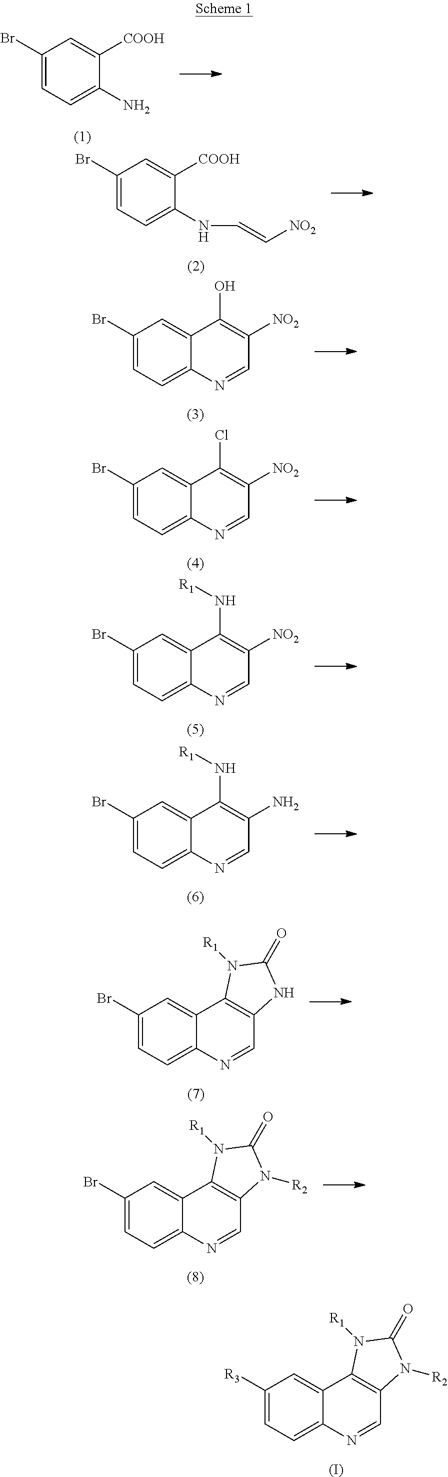

wherein R₁, R₂ and R₃ are as defined in any one of the embodiments of the invention for the compounds of formula (I).

As illustrated in scheme 1, the compound of formula (2), can be prepared by reacting nitromethane in the presence of a base such as NaOH in the temperature range from 0° C. to RT; then adding the product to conc. HCl at about 0-10° C. and adding the compound of the formula (1) in aqueous acid such as water-HCl mixture, and stirring at a temperature ranging from about 0° C. to RT. The nitro compound of formula (2) can be reacted with an acid anhydride such as acetic anhydride in the presence of an alkali metal salt such as potassium acetate or sodium acetate at a temperature ranging from about 80-140° C. to form a compound of formula (3). The nitro-quinolinol compound of formula (3) can be treated with a halogenating agent, for example with a chlorinating agent such as POCl₃ at a temperature ranging from about 80-140° C. to form a compound of formula (4). The compound of formula (4) can be treated with an amine of formula R₁—NH₂ at a temperature range from about 0-40° C. to form a compound of formula (5), wherein R₁ is as defined in any one of the embodiments of the invention for the compounds of formula (I). Catalytic reduction of nitro group of compound of formula (5) forms quinoline-diamine of formula (6). The quinoline-diamine of formula (6) can be reacted with a reagent such as trichloromethylchloroformate or triphosgene in the presence of a base such as triethylamine or trimethylamine in an appropriate solvent such as dichloromethane or chloroform to form a compound of formula (7). The compound of formula (7) can be treated with a compound of formula R₂-hal, wherein hal is halogen and R₂ is as defined in any one of the embodiments of the invention for the compounds of formula (I), in the presence of a base such as sodium hydride to form a compound of formula (8). The compound of formula (8) can be further treated with a compound of formula R₃—B(OH)₂ in the presence of a coupling agent such as palladium dichlorobistriphenylphosphine and a base such as sodium carbonate to form a compound of formula (I), wherein R₁, R₂ and R₃ are as defined in any one of the embodiments of the invention for the compounds of formula (I).

The process of the present invention described herein comprises an optional step of forming a salt and/or a solvate and/or a prodrug of the compound of formula (I).

Isotopically labeled forms of compounds of formula (I) can be prepared by conventional techniques known to those skilled in the art or by processes analogous to those described above and in the subsequent Exemplification section by using an appropriate isotopically labeled reagent instead of non-labeled reagent.

The pharmaceutically acceptable salts of the present invention can be synthesized from the subject compound (the compound of formula I), which contains a basic or an acidic moiety, by conventional chemical methods. Generally the salts are prepared by contacting the free base or acid with an appropriate amount of the desired salt-forming inorganic or organic acid or base in a suitable solvent or dispersant, or by cation or anion exchange. Suitable solvents are, for example, ethyl acetate, ether, alcohols, acetone, tetrahydrofuran, dioxane or mixtures of these solvents. These solvents can also be used for purification of the compounds obtained.

According to a further aspect of the present invention, there is provided a process for the preparation of a compound of formula (I) and its pharmaceutically acceptable salt.

According to a further aspect of the present invention, there is provided a process for the preparation of a compound of formula (7), wherein R₁ is defined in any one of the embodiments of the invention for the compounds of formula (I)

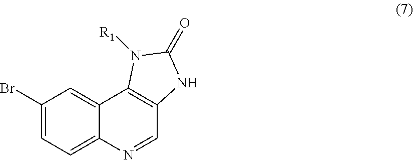

comprising,
reacting a compound of formula (6);

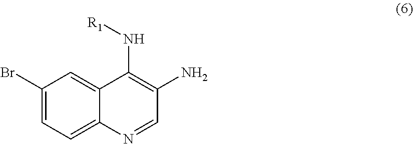

with a reagent such as trichloromethylchloroformate or triphosgene in the presence of a base such as triethylamine or trimethylamine, wherein, R₁ is as defined in any one of the embodiments of the invention for the compounds of formula (I).

According to a further aspect of the present invention, there is provided a process for the preparation of a compound of formula (8), wherein R₁ and R₂ are as defined for formula (I),

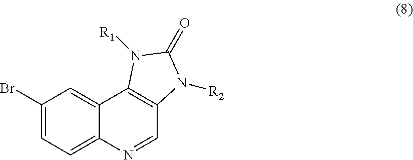

comprising,
reacting a compound of formula (7);

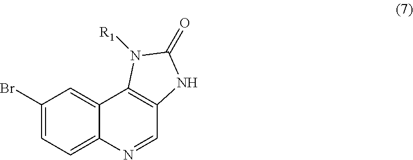

with a compound of formula R₂-hal, wherein hal is halogen and R₂ is as defined in any one of the embodiments of the invention for the compounds of formula (I) in the presence of a base such as sodium hydride, wherein, R₁ is as defined in any one of the embodiments of the invention for the compounds of formula (I).

According to a further aspect of the present invention, there is provided a process for the preparation of a compound of formula (I),

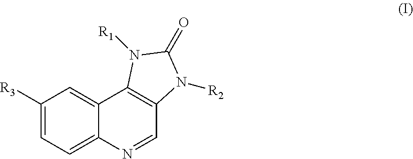

comprising,
reacting a compound of formula (8)

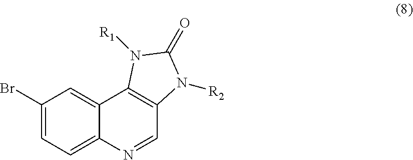

with a compound of formula R₃—B(OH)₂ in the presence of a coupling agent such as palladium dichlorobistriphenylphosphine, wherein R₁ and R₂ are as defined in any one of the embodiments of the invention for the compounds of formula (I).

The compounds of formula (I) can be converted to corresponding pharmaceutically acceptable salts.

### Methods of Treatment

The term “treat or treating or treatment” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorders that damage or interferes with the normal function of a cell, tissue, or organ.

As used herein, the term “therapeutically effective amount” refers to an amount of the compound of formula (I) which, when administered to a subject in need thereof in a proper dosing regimen, is sufficient to treat the target disease or disorder as described herein.

The term “subject” as used herein, refers to an animal, preferably a mammal, most preferably a human, who has been the object of treatment, observation or experiment.

The term “mammal” used herein refers to warm-blooded vertebrate animals of the class mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

Compounds of the present invention inhibit one or more kinases associated with the proliferative diseases or disorders. The kinases associated with proliferative diseases include, but are not limited to PI3K, mTOR, DNA-PK, MAP4K2, ALK1, ALK2, CLK1, CLK4, JAK2, MAP4K5, MuSK, RIPK2 and ROS.

The present invention further provides a method for the treatment of diseases or disorders that can be treated by inhibiting one or more isoforms of PI3K, including PI3Kα, PI3Kβ, PI3Kδ and PI3Kγ.

Proliferative disease or disorder that can be treated by the compounds of formula (I) is cancer, including, but not limited to leukemia such as acute lymphocytic leukemia; acute myeloid leukemia; adult acute myeloid leukemia; acute lymphoblastic leukemia; chronic lymphocytic leukemia; chronic myeloid leukemia; hairy cell leukemia, lung cancer including non-small-cell lung cancer and small-cell lung cancer, brain tumors such as brain stem glioma; glioblastoma; astrocytoma including cerebellar astrocytoma and cerebral astrocytoma; visual pathway and hypothalamic glioma, supratentorial primitive neuroectodermal and pineal tumors; medulloblastoma, lymphoma such as primary central nervous system lymphoma; non-Hodgkin's lymphoma particularly mantle cell lymphoma, Hodgkin's disease, liver cancer such as hepatocellular carcinoma, kidney cancer such as renal cell carcinoma and Wilms' tumor, sarcoma such as Ewing's sarcoma family of tumors; osteosarcoma; Rhabdomyosarcoma; soft tissue sarcomas, mesothelioma, bladder cancer, breast cancer, endometrial cancer, head and neck cancer such as oral cancer; esophageal cancer, melanoma, cervical cancer, thyroid cancer, gastric cancer, germ cell tumor, cholangiocarcinoma, extracranial cancer, malignant fibrous histiocytoma of bone, retinoblastoma, multiple myeloma, pancreatic cancer, ependymoma, neuroblastoma, skin cancer, ovarian cancer, recurrent ovarian cancer, prostate cancer, testicular cancer, colorectal cancer, lymphoproliferative disease, refractory multiple myeloma, resistant multiple myeloma and myeloproliferative disorder, or a combination of one or more of the preceding cancers.

As such, compounds of the present invention can be used to treat tumor cells, and thereby assist in reducing the size of a tumor.

Compounds of the present invention inhibit TNF-α, IL-6 or VEGF associated with inflammatory diseases or disorders and angiogenesis related diseases or disorders.

Inflammatory diseases or disorders that can be treated by the compounds of formula (I) include, but are not limited to, rheumatoid arthritis, juvenile rheumatoid arthritis, psoriatic arthritis, osteoarthritis, refractory rheumatoid arthritis, chronic non-rheumatoid arthritis, osteoporosis, bone resorption, septic shock, Crohn's disease, inflammatory bowel disease, ulcerative colitis, atherosclerosis and psoriasis.

Compounds of the present invention may also be used for the treatment of other diseases or conditions, such as inflammatory or allergic conditions of the skin, for example, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, pemphigus, epidermolysis bullosa acquisita, skin delayed type hypersensitivity disorders; cardiovascular diseases, for example, atherosclerosis, ischaemia-reperfusion injury, coronary heart disease; neural diseases, for example, multiple sclerosis, Alzheimer's disease), sepsis, chronic recurrent uveitis, hepatitis C virus infection, viral infection, bacterial infection, fungal infection, malaria, ulcerative colitis, cachexia, plasmocytoma, endometriosis, Behcet's disease, Wegenrer's granulomatosis, AIDS, HIV infection, autoimmune disease, immune deficiency, common variable immunodeficiency (CVID), chronic graft-versus-host disease, trauma and transplant rejection, adult respiratory distress syndrome, pulmonary fibrosis, chronic obstructive pulmonary disease, bronchitis, metabolic disorders (such as diabetes and juvenile diabetes), meningitis, ankylosing spondylitis, systemic lupus erythematosus, allergic asthma, inflammation, septic shock, endotoxic shock, vasculitis and amyloidosis.

Compounds of the present invention can be used for the treatment of angiogenesis related diseases or disorders.

Compounds of the present invention may also be used for the treatment of diseases in which angiogenesis is believed to be important, referred to as angiogenic diseases, including but not limited to, inflammatory disorders such as immune and non-immune inflammation, chronic articular rheumatism, psoriasis, disorders associated with inappropriate or inopportune invasion of vessels such as diabetic retinopathy, neovascular glaucoma, capillary proliferation in atherosclerotic plaques and osteoporosis, and cancer associated disorders, such as solid tumors, solid tumor metastases, angiofibromas, retrolental fibroplasia, hemangiomas, Karposi's sarcoma and the like cancers which require neovascularization to support tumor growth.

The following abbreviations and definitions are used throughout this application:

The term “tumor” as used herein refers to an abnormal growth of tissue resulting from uncontrolled, progressive multiplication of cells. A tumor can be benign or malignant.

### ABBREVIATIONS

PI3 kinase phosphatidylinositol-3-kinase
mTOR mammalian target of rapamycin
DNA-PK DNA-dependent protein kinase
MAP4K2 mitogen-activated protein kinase kinase kinase kinase 2
ALK1 (also known as ACVRL1) activin receptor-like kinase 1
ALK2 (also known as ACVR1) activin A receptor, type I
CLK1 CDC-like kinase 1
CLK4 CDC-like kinase 4
JAK2 Janus kinase 2
MAP4K5 mitogen-activated protein kinase kinase kinase kinase 5
MuSK muscle-specific receptor tyrosine kinase
RIPK2 receptor-interacting serine/threonine-protein kinase 2
ROS reactive oxygen species

According to another aspect of the present invention, there is provided a method of treating diseases or disorders selected from proliferative diseases, inflammatory diseases or disorders or angiogenesis related diseases or disorders in a subject, comprising administering to the subject a therapeutically effective amount of a compound of formula (I) or a stereoisomer or a tautomer, or an N-oxide or a pharmaceutically acceptable salt or a solvate thereof.

According to another aspect of the present invention, there is provided a method of treating diseases or disorders mediated by one or more kinases selected from CLK-1, CLK-4, DNA-PK, MAP4K2, MAP4K5 or RIPK2 in a subject, comprising administering to the subject a therapeutically effective amount of a compound of formula (I) or a pharmaceutically acceptable salt thereof.

According to another aspect of the present invention, there is provided a method of treating diseases or disorders mediated by one or more kinases selected from PI3K, mTOR, ALK-1 or ALK-2 in a subject, comprising administering to the subject a therapeutically effective amount of a compound of formula (I) or a pharmaceutically acceptable salt thereof.

According to another aspect of the present invention there is provided a method of inhibiting activity of kinases selected from PI3K, mTOR, ALK-1 or ALK-2 comprising contacting the kinase with an effective amount of a compound of formula (I).

According to another aspect of the present invention, there is provided a method of treating diseases mediated by VEGF in a subject, comprising administering to the subject a therapeutically effective amount of a compound of formula (I) or a pharmaceutically acceptable salt thereof.

According to another aspect of the present invention there is provided a method of inhibiting VEGF, comprising contacting VEGF with an effective amount of a compound of formula (I).

According to further aspect of the present invention, there is provided a method for the treatment of diseases mediated by TNF-α or IL-6 in a subject, comprising administering to the subject a therapeutically effective amount of a compound of formula (I) or a pharmaceutically acceptable salt thereof.

According to another aspect of the present invention there is provided a method for inhibiting TNF-α or IL-6, comprising contacting TNF-α or IL-6 with an effective amount of a compound of formula (I).

According to another aspect of the present invention, there is provided a method of treating proliferative diseases or disorders, inflammatory diseases or disorders or angiogenesis related diseases or disorders mediated by one or more kinases selected from PI3 kinase, mTOR, ALK-1 or ALK-2, comprising administering to a subject in need thereof a therapeutically effective amount of a compound of formula (I) or a pharmaceutically acceptable salt thereof.

According to another aspect of the present invention, there is provided a method for the treatment of proliferative diseases or disorders mediated by one or more kinases, selected from PI3K, mTOR, ALK-1 or ALK-2 in a subject, comprising administering to the subject a therapeutically effective amount of a compound of formula (I) or a pharmaceutically acceptable salt thereof.

According to another aspect of the present invention, there is provided a compound of formula (I) or a stereoisomer, a tautomer, an N-oxide, a pharmaceutically acceptable salt or a solvate thereof, for use in the treatment of diseases or disorders selected from proliferative diseases or disorders, inflammatory diseases or disorders or angiogenesis related diseases or disorders.

According to another aspect of the present invention, there is provided a compound of formula (I) or a pharmaceutically acceptable salt thereof for use in the treatment of diseases or disorders mediated by one or more kinases selected from CLK-1, CLK-4, DNA-PK, MAP4K2, MAP4K5 or RIPK2.

According to another aspect of the present invention, there is provided a compound of formula (I) or a pharmaceutically acceptable salt thereof for use in the treatment of diseases or disorders mediated by one or more kinases selected from PI3K, mTOR, ALK-1 or ALK-2.

According to further aspect of the present invention, there is provided a compound of formula (I) or a pharmaceutically acceptable salt thereof for use in the treatment of diseases mediated by TNF-α or IL-6.

According to another aspect of the present invention, there is provided a compound of formula (I) or a pharmaceutically acceptable salt thereof for use in the treatment of diseases mediated by VEGF.

According to another aspect of the present invention, there is provided a compound of formula (I) or a pharmaceutically acceptable salt thereof for use in the treatment of proliferative diseases, inflammatory diseases or angiogenesis related disorders mediated by one or more kinases selected from PI3K, mTOR, ALK-1 or ALK-2.

According to another aspect of the present invention, there is provided a compound of formula (I) or a pharmaceutically acceptable salt thereof for use in the treatment of proliferative diseases or disorders mediated by one or more kinases, such as PI3K, mTOR, ALK-1 or ALK-2.

According to another aspect of the present invention, the proliferative disease mediated by one or more kinases is cancer.

According to another aspect of the present invention, the cancer is solid cancer or hematological cancer.

According to another embodiment of the present invention, the cancer is selected from: leukemia such as acute lymphocytic leukemia; acute myeloid leukemia; adult acute myeloid leukemia; acute lymphoblastic leukemia; chronic lymphocytic leukemia; chronic myeloid leukemia; hairy cell leukemia, lung cancer including non-small-cell lung cancer and small-cell lung cancer, brain tumors such as brain stem glioma; glioblastoma; astrocytoma including cerebellar astrocytoma and cerebral astrocytoma, visual pathway and hypothalamic glioma; supratentorial primitive neuroectodermal and pineal tumors; medulloblastoma, lymphoma such as primary central nervous system lymphoma; non-Hodgkin's lymphoma particularly mantle cell lymphoma, Hodgkin's disease, liver cancer such as hepatocellular carcinoma, kidney cancer such as renal cell carcinoma and Wilms' tumor, sarcoma such as Ewing's sarcoma family of tumors; osteosarcoma; rhabdomyosarcoma; soft tissue sarcomas, mesothelioma, bladder cancer, breast cancer, endometrial cancer, head and neck cancer, melanoma, cervical cancer, thyroid cancer, gastric cancer, germ cell tumor, cholangiocarcinoma, extracranial cancer, malignant fibrous histiocytoma of bone, retinoblastoma, esophageal cancer, multiple myeloma, oral cancer, pancreatic cancer, ependymoma, neuroblastoma, skin cancer, ovarian cancer, recurrent ovarian cancer, prostate cancer, testicular cancer, colorectal cancer, lymphoproliferative disease, refractory multiple myeloma, resistant multiple myeloma or myeloproliferative disorder, or a combination of one or more of the preceding cancers.

According to another embodiment of the present invention, the cancer is selected from leukemia, lung cancer, brain tumors, Hodgkin's disease, liver cancer, kidney cancer, bladder cancer, breast cancer, head and neck cancer, endometrial cancer, lymphoma, melanoma, cervical cancer, thyroid cancer, gastric cancer, germ cell tumor, cholangiocarcinoma, extracranial cancer, sarcoma, mesothelioma, malignant fibrous histiocytoma of bone, retinoblastoma, esophageal cancer, multiple myeloma, oral cancer, pancreatic cancer, neuroblastoma, skin cancer, ovarian cancer, recurrent ovarian cancer, prostate cancer, testicular cancer, colorectal cancer, lymphoproliferative disease, refractory multiple myeloma, cancer of urinary tract, resistant multiple myeloma or myeloproliferative disorder.

According to another embodiment of the present invention, the cancer is selected from breast cancer, prostate cancer, pancreatic cancer, lung cancer, head and neck cancer, ovarian cancer, colorectal cancer, kidney cancer, gastric cancer, non-Hodgkin's lymphoma, primary central nervous system lymphoma, endometrial cancer, brain tumor, melanoma, liver cancer, thyroid cancer, lymphoid cancer, esophageal cancer, cancer of urinary tract, cervical cancer, bladder cancer, mesothelioma, sarcoma or chronic myeloid leukemia.

According to another embodiment of the present invention, the cancer is selected from ovarian cancer, prostate cancer, breast cancer, pancreatic cancer, brain tumors or chronic myeloid leukemia.

According to another aspect of the present invention, there is provided a method for the treatment of inflammatory diseases or disorders mediated by one or more kinases, including, but not limited to, PI3K and mTOR, comprising administering to a subject in need thereof a therapeutically effective amount of a compound of formula (I) or a pharmaceutically acceptable salt thereof.

According to further aspect of the present invention, there is provided a method for the treatment of inflammatory diseases mediated by TNF-α or IL-6 in a subject, comprising administering to the subject a therapeutically effective amount of a compound of formula (I) or a pharmaceutically acceptable salt thereof.

According to another aspect of the present invention, there is provided a compound of formula (I) or a pharmaceutically acceptable salt thereof for use in the treatment of inflammatory diseases or disorders mediated by one or more kinases, including, but not limited to, PI3K and mTOR.

According to further aspect of the present invention, there is provided a compound of formula (I) or a pharmaceutically acceptable salt thereof for use in the treatment of inflammatory diseases or disorders mediated by TNF-α or IL-6.

According to another aspect of the present invention, the inflammatory diseases or disorders are selected from rheumatoid arthritis, Crohn's disease, ulcerative colitis, inflammatory bowel disease, chronic non-rheumatoid arthritis, osteoporosis, septic shock, psoriasis or atherosclerosis.

According to another aspect of the present invention, there is provided a method of treating angiogenesis related disorders mediated by one or more kinases, including but not limited to, PI3K, mTOR, ALK-1 or ALK-2 in a subject, comprising administering to the subject a therapeutically effective amount of a compound of formula (I) or a pharmaceutically acceptable salt thereof.

According to another aspect of the present invention, there is provided a method of treating angiogenesis related disorders mediated by VEGF in a subject, comprising administering to the subject a therapeutically effective amount of a compound of formula (I) or a pharmaceutically acceptable salt thereof.

According to another aspect of the present invention, there is provided a compound of formula (I) or a pharmaceutically acceptable salt thereof for use in the treatment of angiogenesis related disorders mediated by one or more kinases, including but not limited to, PI3K, mTOR, ALK-1 or ALK-2.

According to another aspect of the present invention, there is provided a compound of formula (I) or a pharmaceutically acceptable salt thereof; for use in the treatment of angiogenesis related disorders mediated by VEGF.

According to another aspect of the present invention, the angiogenesis related disorder is an inflammatory disorder.

According to another aspect of the present invention, the inflammatory disorder which is an angiogenesis related disorder is selected from immune and non-immune inflammation, chronic articular rheumatism, disorders associated with inappropriate or inopportune invasion of vessels such as diabetic retinopathy, neovascular glaucoma, capillary proliferation in atherosclerotic plaques or osteoporosis.

According to another aspect of the present invention, the angiogenesis related disorder is cancer associated disorder, such as solid tumor, solid tumor metastasis, angiofibroma, retrolental fibroplasia, hemangioma or Kaposi's sarcoma.

According to another aspect of the present invention, the anti-angiogenic potential of the compounds of the present invention can be determined using zebra fish assay by following the protocol as described in Nature Protocols, 2007, 2, 2918-2923.

According to another aspect of the present invention, there are provided methods for the manufacture of medicaments comprising compounds of formula (I) or pharmaceutically acceptable salts thereof, which are useful for the treatment of cancers.

According to another aspect of the present invention, there are provided methods for the manufacture of medicaments, comprising compounds of formula (I) or pharmaceutically acceptable salts thereof, which are useful for the treatment of angiogenesis related diseases or disorders.

According to another aspect of the present invention, there are provided methods for the manufacture of medicaments, comprising compounds of formula (I) or pharmaceutically acceptable salts thereof, which are useful for the treatment of inflammatory diseases or disorders.

Additionally, the present invention provides a compound of formula (I) or a stereoisomer, a tautomer, an N-oxide or a pharmaceutically acceptable salt thereof, for use in the treatment of the human or animal body.

### Pharmaceutical Compositions

The pharmaceutical preparations according to the invention are prepared in a manner known per se and familiar to one skilled in the art. Pharmaceutically acceptable inert inorganic and/or organic carriers and/or additives can be used in addition to the compounds of formula (I), and/or their pharmaceutically acceptable salts. For the production of pills, tablets, coated tablets and hard gelatin capsules it is possible to use, for example, lactose, corn starch or derivatives thereof, gum acacia, magnesia or glucose, etc. Carriers for soft gelatin capsules and suppositories are, for example, fats, waxes, natural or hardened oils, etc. Suitable carriers for the production of solutions, for example injection solutions, or for emulsions or syrups are, for example, water, physiological sodium chloride solution or alcohols, for example, ethanol, propanol or glycerol, sugar solutions, such as glucose solutions or mannitol solutions, or a mixture of the various solvents which have been mentioned.

The pharmaceutical preparations normally contain about 1 to 99%, for example, about 5 to 70%, or from about 5 to about 30% by weight of the compound of formula (I) or pharmaceutically acceptable salt thereof. The amount of the compound of formula (I) or pharmaceutically acceptable salt thereof in the pharmaceutical preparations normally is from about 1 to 1000 mg.

The dose of the compounds of this invention, which is to be administered, can cover a wide range. The dose to be administered daily is to be selected to produce the desired effect. A suitable dosage is about 0.01 to 100 mg/kg of the compound of formula (I) or pharmaceutically acceptable salt thereof, for example, about 0.01 to 20 mg/kg of a compound of formula (I) or a pharmaceutically acceptable salt thereof, with the typical dose being about 0.1 to 5 mg/kg of a compound of formula (I) or a pharmaceutically acceptable salt thereof. If required, higher or lower daily doses can also be administered. Actual dosage levels of the active ingredients in the pharmaceutical compositions of this invention may be varied so as to obtain an amount of the compound of formula (I), which is effective to achieve the desired therapeutic response for a particular subject.

The pharmaceuticals can be administered orally, for example in the form of pills, tablets, coated tablets, lozenges, capsules, dispersible powders or granules, suspensions, emulsions, syrups or elixirs. Administration, however, can also be carried out rectally, for example in the form of suppositories, or parenterally, for example intravenously, intramuscularly or subcutaneously, in the form of injectable sterile solutions or suspensions, or topically, for example in the form of solutions or ointments or transdermally, for example in the form of transdermal patches, or in other ways, for example in the form of aerosols, nasal sprays or nasal drops.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts.

In addition to the compound of formula (I) and/or its pharmaceutically acceptable salt and carrier substances, the pharmaceutical preparations can contain additives such as, for example, fillers, antioxidants, dispersants, emulsifiers, defoamers, flavors, preservatives, solubilizers or colorants. They can also contain one or more compounds of formula (I) and/or their pharmaceutically acceptable salts. Furthermore, in addition to at least one compound of formula (I) and/or its pharmaceutically acceptable salt, the pharmaceutical preparations can also contain one or more other therapeutically or prophylactically active ingredients.

By “pharmaceutically acceptable” it is meant the carrier, diluent, excipients, and/or salt must be compatible with the other ingredients of the formulation, and not deleterious to the recipient thereof.

According to another aspect of the present invention there is provided a pharmaceutical composition, comprising a therapeutically effective amount of a compound of formula (I) or a pharmaceutically acceptable salt thereof in association with a pharmaceutically acceptable excipient or carrier.

According to another aspect of the present invention there is provided any of the uses, methods or compositions as defined above wherein the compound of formula (I), or pharmaceutically acceptable salt thereof, is used in combination with another pharmacologically active compound, particularly one of the compounds listed herein below.

A compound of formula (I) may be administered either simultaneously or before or after the pharmacologically active compound, either separately by the same or different route of administration, or together in the same pharmaceutical formulation.

According to another aspect of the present invention there is provided a pharmaceutical composition, comprising a therapeutically effective amount of a compound of formula (I) or a pharmaceutically acceptable salt or a pharmaceutically acceptable solvate thereof and at least one further pharmaceutically active compound, together with a pharmaceutically acceptable excipient or carrier. A pharmaceutically active compound in combination with one or more compounds of formula (I) for treatment of cancer can be selected from, but not limited to, one or more of the following groups: (i) Kinase inhibitors such as gefitinib, imatinib, erlotinib, lapatinib, bevacizumab (avastin), sorafenib, Bcr-Abl kinase inhibitors or LY-317615 (ii) Alkylating agents such as mitomycin C, busulfan, oxaliplatin, cisplatin, carboplatin, procarbazine or dacarbazine (iii) Antimetabolites such as methotrexate, mercaptopurine, thioguanine, fludarabine phosphate, fluorouracil, vinblastine, vincristine, gemcitabine or paclitaxel (iii) Antibiotics such as anthracyclines, dactinomycin or bleomycin (iv) Hormonal agents such as tamoxifen, flutamide, GnRH (Gonadotropin-Releasing Hormone) agonists or aromatase inhibitors or (v) Cancer vaccines such as avicine, oregovomab or theratope.

It is understood that modifications that do not substantially affect the activity of the various embodiments of this invention are included within the invention disclosed herein. Accordingly, the following examples are intended to illustrate but not to limit the present invention.

### Exemplification

### Synthetic Methods

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims. For example, the synthesis of non-exemplified compounds according to the invention may be successfully performed by modifications apparent to those skilled in the art.

Nomenclature of the compounds exemplified in the present invention was derived from Chemdraw Ultra version 9.0.1 CambridgeSoft Corporation, Cambridge.

Reagents were purchased from commercial suppliers such as Sigma Aldrich Chemical company, Spectrochem Ltd., India; AK scientific Inc. CA, Thomas Baker (Chemicals) Pvt. Ltd., India; Merck KgaA, Darmstadt, Germany and are used as such.

Unless otherwise stated all temperatures are in degree Celsius. Also, in these examples and elsewhere, abbreviations have the following meanings:

List of abbreviations

| | |
|---|---|
| ATCC | American Type Culture Collection |
| ATP | Adenosine triphosphate |
| BSA | Bovine Serum Albumin |
| CO₂ | Carbon dioxide |
| CHCl₃ | Chloroform |
| CDCl₃ | Deuterated chloroform |
| cpm | Counts per minute |
| DCM | Dichloromethane |
| DMF | Dimethyl formamide |
| DMSO | Dimethyl sulfoxide |
| DTT | Dithiothreeitol |
| EDTA | Ethylene Diamine Tetraacetic Acid |
| EGTA | Ethylene Glycol Tetraacetic Acid |
| EtOAc | Ethyl acetate |
| ELISA | Enzyme-Linked Immunosorbent Assay |
| FCS | Fetal Calf Serum |
| g | Gram |
| HCl | Hydrochloric acid |
| IL-6 | Interleukin 6 |
| IFN-γ | Interferon-γ |
| MgCl₂ | Magnesium chloride |
| MeOH | Methanol |
| μg | microgram |
| μL | microliter |
| mL | Milliliter |
| mM or mmol | milliMolar |
| mg | milligram |
| GST | glutathione S-Transferase |
| μM | micro Molar |
| ng | nanogram |
| nM | nanoMolar |
| pM | picoMolar |
| MOPSO | 3-(N-Morpholino)-2-hydroxypropanesulfonic |
| | Acid |
| NaCl | Sodium chloride |
| NaH | Sodium hydride |
| Na₂CO₃ | Sodium carbonate |
| NaF | Sodium Fluoride |
| NaHCO₃ | Sodium bicarbonate |
| NaOH | Sodium hydroxide |
| Na₂SO₄ | Sodium sulfate |
| Ni | Nickel |
| NP-40 | Nonidet P40 |
| psi | pound per square inch |
| POCl₃ | Phosphorus oxychloride |
| PBS | Phosphate buffer saline |
| RT | Room Temperature (20-30° C.) |
| RPMI | Roswell Park Memorial Institute |
| SDS-PAGE | Sodium dodecyl sulfate Polyacrylamide Gel |
| | Electrophoresis |
| TBS | Tris Buffered Saline |
| TBST | Tween Tris buffer saline |
| THF | Tetrahydrofuran |
| TNF-α | Tumor necrosis factor α |
| VEGF | Vascular Endothelial Growth Factor |

### Intermediates

### Intermediate 1: 6-bromo-4-chloro-3-nitroquinoline

### A: 5-bromo-2-(2-nitrovinylamino)benzoic acid

A suspension of 2-amino-5-bromobenzoic acid (231 mmol) in water-HCl (37%) (10:1) was stirred for 8 hours and was filtered (solution 1). Nitromethane (278 mmol) was added over 10 minutes to a mixture of ice (70 g) and NaOH (775 mmol) at 0° C. under stirring. After stirring for 1 hour at 0° C. and 1 hour at RT, this solution was added to a mixture of ice (56 g) and 84 mL of HCl (37%) at 0° C. (solution 2). Solution 1 and 2 were combined and the reaction mixture was stirred for 18 hours at RT. The yellow precipitate was filtered, washed with water and dried at 40° C. to obtain the title compound. The crude product was used directly for the next step. Yield: 38%.

### B: 6-bromo-3-nitroquinolin-4-ol

5-bromo-2-(2-nitrovinylamino)benzoic acid (Compound A, 87 mmol) and potassium acetate (104 mmol) in acetic anhydride (1185 mmol) were stirred for 3 hours at 120° C. The precipitate was filtered, and washed with acetic acid until the filtrate was colorless. It was further washed with water and dried to obtain the title compound. ¹H NMR (500 MHz, CDCl₃): δ 9.275 (s, 1H), 8.611-8.615 (d, 1H, J=2 Hz), 8.100-8.118 (d, 1H, J=9 Hz), 8.026-8.048 (dd, 1H, J=8.5 Hz, 2 Hz).

### C: 6-bromo-4-chloro-3-nitroquinoline

6-bromo-3-nitroquinolin-4-ol (compound B, 74.3 mmol) and POCl₃ (1613 mmol) were stirred for 45 minutes at 120° C. The mixture was cooled to RT and poured slowly into ice-water. The precipitate was filtered, washed with ice-cold water, and dissolved in DCM. The organic layer was washed with cold brine, and was dried over Na₂SO₄. The solvent was evaporated to dryness to obtain the title compound. The crude product was used directly for the next step.

### Intermediate 2: 2-(5-aminopyridin-2-yl)-2-methylpropanenitrile

### A: 2-methyl-2-(5-nitropyridin-2-yl)propanenitrile

Sodium hydride (67.44 mmol) was added to a solution of 2-(5-nitropyridine-2-yl)acetonitrile (30.65 mmol) in dry THF (250 mL) at 0° C. and the reaction mixture was stirred for 0.5 hours. Methyl iodide (91.95 mmol) was added to the reaction mixture and reaction mixture was warmed to RT, stirred for another 24 hours. Solvent was removed under vacuum; crude product was purified (silica gel column, EtOAc/hexane as eluent) to obtain the title compound. ¹H NMR (300 MHz, DMSO-d₆): δ 9.43 (d, J=2.7 Hz, 1H), 8.55 (dd, J=8.7, 2.4 Hz, 1H), 7.86 (d, J=8.7 Hz, 1H), 1.82 (s, 6H); MS (m/z): 192 (M+1)⁺.

### B: 2-(5-aminopyridin-2-yl)-2-methylpropanenitrile

2-methyl-2-(5-nitropyridin-2-yl)propanenitrile (15.70 mmol) was subjected to hydrogenation using Raney-Ni (0.6 g) at 40 psi for 4 hours. Reaction mixture was filtered and washed with methanol. Filtrate was concentrated and purified (silica gel column, MeOH/CHCl₃ as eluent) to obtain the title compound. ¹H NMR (300 MHz, DMSO-d₆): δ 8.11 (d, J=2.7 Hz, 1H), 7.37 (d, J=8.7 Hz, 1H), 7.03 (dd, J=2.7, 8.7 Hz, 1H), 3.36 (brs, 2H), 1.74 (s, 6H); MS (m/z): 162 (M+1)⁺.

### Method for Preparation of Salts

### Method A: General Method for Preparation of Mesylate Salts

A solution of compound of formula (I) (0.106 mmol) in dry dichloromethane (5 mL) was stirred at 0° C. Methane sulfonic acid (0.01021 g, 0.106 mmol) dissolved in dry dichloromethane (1 mL) was added drop-wise to the solution of the compound over a period of 0.5 hours. Reaction mixture was stirred at same temperature for 0.5 hours, warmed to RT room temperature, and stirred further for 4 hours. Solvent was removed and the mesylate salt of the compound of formula (I) was obtained. The salt so obtained was characterized by NMR.

### Method B: General Method for Preparation of Hydrochloride Salts

A solution of compound of formula (I) (0.106 mmol) in dry dichloromethane (5 mL) was stirred at 0° C. Ethereal HCl was added in excess to the solution of the compound. Reaction mixture was stirred at same temperature for 0.5 hours, warmed to RT and further stirred for 4 hours. Solvent was removed and the hydrochloride salt of the compound of formula (I) was obtained. The salt so obtained was characterized by NMR.

### EXAMPLES

### Example 1

### 2-Methyl-2-(5-(3-methyl-2-oxo-8-(pyridin-3-yl)-2,3-dihydro-1H-imidazo[4,5-c]quinolin-1-yl)pyridin-2-yl)propanenitrile

### Step 1: 2-(5-(6-Bromo-3-nitroquinolin-4-ylamino)pyridin-2-yl)-2-methylpropanenitrile

6-bromo-4-chloro-3-nitroquinoline (Intermediate 1, 5.2 mmol) and 2-(5-aminopyridin-2-yl)-2-methylpropanenitrile (Intermediate 2, 5.2 mmol) were dissolved in acetic acid (5 mL) and the mixture was stirred overnight. Water was added and the yellow precipitate was filtered off. The precipitate was washed with water and dried. The solid obtained was partitioned and extracted with EtOAc and THF, washed with saturated aqueous NaHCO₃. The organic layer was dried over anhydrous sodium sulfate and concentrated to obtain the title compound. ¹H NMR (300 MHz, DMSO-d₆): δ 10.17 (s, 1H), 9.12 (s, 1H), 8.73 (s, 1H), 8.44 (s, 1H), 8.06 (d, J=10.2 Hz, 1H), 7.97 (d, J=8.7 Hz, 1H), 7.53 (s, 2H), 1.70 (s, 6H); MS (m/z): 412.0 (M−1)⁻.

### Step 2: 2-(5-(3-Amino-6-bromoquinolin-4-ylamino)pyridin-2-yl)-2-methylpropanenitrile

2-(5-(6-bromo-3-nitroquinolin-4-ylamino)pyridin-2-yl)-2-methylpropanenitrile (compound of step 1, 13.3 mmol) was reduced using Raney-Ni (1 g) in THF-MeOH [(1:1), 50 mL] under 40 psi of hydrogen for 4 hours at RT. After completion of the reaction, the reaction mixture was filtered and washed with methanol. The filtrate was concentrated and purified (silica gel column, MeOH/CHCl₃ as eluent) to obtain the title compound. ¹H NMR (300 MHz, DMSO-d₆): δ 8.63 (s, 1H), 8.19 (s, 1H), 8.04 (d, J=2.4, 1H), 7.89 (d, J=2.4, 1H), 7.80 (d, J=8.7 Hz, 1H), 7.49 (dd, J=2.1, 8.7 Hz, 1H), 7.33 (d, J=8.7 Hz, 1H), 6.67 (dd, J=2.7, 8.4 Hz, 1H), 5.59 (s, 2H), 1.64 (s, 6H); MS (m/z): 384 (M+1)⁺.

### Step 3: 2-(5-(8-Bromo-2-oxo-2,3-dihydro-1H-imidazo[4,5-c]quinolin-1-yl)pyridin-2-yl)-2-methylpropanenitrile

A solution of 2-(5-(3-amino-6-bromoquinolin-4-ylamino)pyridin-2-yl)-2-methylpropanenitrile (compound of step 2, 3.48 mmol) and triethylamine (15.7 mmol) in DCM (25 mL) was added to a solution of triphosgene (4.17 mmol) in DCM (25 mL) for about 40 minutes at 0° C. The reaction mixture was stirred for 30 minutes and then quenched with saturated aqueous NaHCO₃, stirred for 5 minutes and extracted with DCM. The organic layer was dried over Na₂SO₄, filtered and solvent was evaporated to obtain the title compound. ¹H NMR (DMSO-d₆; 300 MHz): δ 11.97 (s, 1H), 8.90 (s, 1H), 8.83 (s, 1H), 8.31-8.23 (m, 1H), 7.98-7.95 (m, 2H), 7.69 (d, J=9.0 Hz, 1H), 6.99 (s, 1H), 1.8 (s, 6H); MS (m/z): 408 (M+1)⁺.

### Step 4: 2-(5-(8-Bromo-3-methyl-2-oxo-2,3-dihydro-1H-imidazo[4,5-c]quinolin-1-yl)pyridin-2-yl)-2-methylpropanenitrile

To a solution of 2-(5-(8-bromo-2-oxo-2,3-dihydro-1H-imidazo[4,5-c]quinolin-1-yl)pyridin-2-yl)-2-methylpropanenitrile (compound of step 3, 0.674 mmol) in 5 mL of dry DMF at 0° C. was added NaH (60% dispersed in mineral oil, 1.482 mmol). The reaction mixture was stirred for 15 minutes followed by addition of methyl iodide (0.741 mmol). Reaction mixture was stirred at 0° C. for another 1 hour and quenched with water. The solvent was removed; aqueous layer was extracted with DCM. Organic layer was dried over anhydrous Na₂SO₄, concentrated under vacuum and purified (silica gel column, MeOH/CHCl₃ as eluent) to obtain the title compound. ¹H NMR (300 MHz, DMSO-d₆): δ 9.09 (s, 1H), 8.91 (d, J=2.4 Hz, 1H), 8.25 (dd, J=2.4, 8.4 Hz, 1H), 8.02-7.96 (m, 2H), 7.70 (dd, J=2.1, 9.3 Hz, 1H), 6.99 (d, J=1.8 Hz, 1H), 3.61 (s, 3H), 1.83 (s, 6H); MS (m/z): 422.1 (M+1)⁺.

### Step 5: 2-Methyl-2-(5-(3-methyl-2-oxo-8-(pyridin-3-yl)-2,3-dihydro-1H-imidazo[4,5-c]quinolin-1-yl)pyridin-2-yl)propanenitrile

Pyridin-3-ylboronic acid (1.233 mmol) and palladium dichlorobis triphenylphosphine (10 mol %) were added to a solution of 2-(5-(8-bromo-3-methyl-2-oxo-2,3-dihydro-1H-imidazo[4,5-c]quinolin-1-yl)pyridin-2-yl)-2-methylpropanenitrile (compound of step 4, 1.118 mmol) in dry DMF (3 mL) in an inert atmosphere. Saturated Na₂CO₃ (0.3 mL) was added to the reaction mixture and the resulting solution was heated at 110° C. for 3 hours. The solvent was removed; the crude material was extracted in EtOAc, washed with brine and dried over anhydrous Na₂SO₄. The solvent was evaporated and the crude solid was purified (silica gel column, EtOAc/MeOH as eluent) to obtain the title compound. ¹H NMR (300 MHz, DMSO-d₆): δ 8.9 (s, 1H), 8.86 (d, J=2.1 Hz, 1H), 8.65 (s, 1H), 8.60 (d, J=4.2 Hz, 1H), 8.28 (d, J=9 Hz, 1H), 8.02 (dd, J=2.4, 8.4 Hz, 1H), 7.93 (d, J=8.4 Hz, 1H), 7.86 (dd, J=8.7, 2.4 Hz, 1H), 7.68-7.66 (m, 1H), 7.65 (dd, J=4.8, 7.8 Hz, 1H), 7.26 (d, J=1.8 Hz, 1H), 3.75 (s, 3H), 1.90 (s, 6H); MS (m/z): 421 (M+1)⁺.

The compounds of Example 2 and 3 were prepared by following the procedure as described for Example 1, using the compound of step 4, and an appropriate boronic acid derivative.

### Example 2

### 2-Methyl-2-(5-(3-methyl-2-oxo-8-(quinolin-3-yl)-2,3-dihydro-1H-imidazo[4,5-c]quinolin-1-yl)pyridin-2-yl)propanenitrile

¹H NMR (300 MHz, CDCl₃): 8.93 (d, J=2.1 Hz, 1H), 8.91 (s, 1H), 8.37 (d, J=8.7 Hz, 1H), 8.16 (s, 1H), 8.13 (s, 1H), 8.05-7.94 (m, 3H), 7.89 (d, J=7.5 Hz, 2H), 7.77-7.71 (m, 1H), 7.67-7.60 (m, 1H), 7.41 (d, J=1.8 Hz, 1H), 3.76 (s, 3H), 1.87 (s, 6H); MS (m/z): 471.1 (M+1)⁺.

### Example 3

### 2-(5-(8-(6-Amino-5-(trifluoromethyl)pyridin-3-yl)-3-methyl-2-oxo-2,3-dihydro-1H-imidazo[4,5-c]quinolin-1-yl)pyridin-2-yl)-2-methylpropanenitrile

¹H NMR (300 MHz, DMSO-d₆): δ 9.03 (s, 1H), 8.95 (d, J=2.1 Hz, 1H), 8.31 (d, J=2.4 Hz, 1H), 8.28 (s, 1H), 8.13 (d, J=9.0 Hz, 1H), 7.96-7.91 (m, 2H), 7.66 (d, J=1.5 Hz, 1H), 7.09 (d, J=1.2 Hz, 1H), 6.71 (s, 2H), 3.62 (s, 3H), 1.81 (s, 6H); MS (m/z): 504.1 (M+1)⁺.

### Example 3a

### 8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium methanesulfonate

The title compound was prepared by following the General method for preparation of mesylate salts as described in method A, using compound of Example 3. ¹H NMR (300 MHz, DMSO-d₆): δ 9.37 (s, 1H), 8.98 (d, J=2.4 Hz, 1H), 8.34 (m, 1H), 8.28 (brs, 1H), 8.23 (brs, 2H), 8.00 (d, J=8.4 Hz, 1H), 7.71 (s, 1H), 7.18 (s, 1H), 6.90 (brs, 1H), 3.67 (s, 3H), 2.33 (s, 3H), 1.82 (s, 6H); MS (m/z): 504.1 (M+1)⁺.

### Example 3b

### 8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium chloride

The title compound was prepared by following the General method for preparation of hydrochloride salts as described in method B, using compound of Example 3. ¹H NMR (300 MHz, DMSO-d₆): δ 9.50 (s, 1H), 9.00 (d, J=2.1 Hz, 1H), 8.46 (d, J=9.0 Hz, 1H), 8.37-8.33 (m, 2H), 8.30 (s, 1H), 8.03 (d, J=8.7 Hz, 1H), 7.74 (bs, 1H), 7.20 (bs, 1H), 7.10-6.80 (bp, 2H), 3.68 (s, 3H), 1.81 (s, 6H); MS (m/z): 504.1 (M+1)⁺.

The compounds of Examples 4-13 were prepared by following the procedure as described for Example 1, using an appropriate boronic acid derivative.

| Ex. No. | Nomenclature | NMR/Mass |
|---|---|---|
| 4 | 2-Methyl-2-(5-(3-methyl- | ¹H NMR (300 MHz, DMSO-d₆): δ 7.28(d, J = 1.8 |
| | 2-oxo-8-(5- | Hz, 1H), 3.64(s, 3H), 1.81 (s, 6H); |
| | (trifluoromethyl)pyridin-3- | MS (m/z): 489.2(M + 1)⁺ |
| | yl)-2,3-dihydro-1H- | |
| | imidazo[4,5-c]quinolin-1- | |
| | yl)pyridin-2-yl) | |
| | propanenitrile | |
| 5 | 2-Methyl-2-(5-(3-methyl- | ¹H NMR (300 MHz, DMSO-d₆): δ 9.07 (s, 1H), |
| | 2-oxo-8-(quinolin-6-yl)- | 9.00 (d, J = 2.4 Hz, 1H), 8.90 (bd, J = 3.0 Hz, 1H), |
| | 2,3-dihydro-1H- | 8.40-8.32 (m, 2H), 8.21 (d, J = 9.0 Hz, 1H), |
| | imidazo[4,5-c]quinolin-1- | 8.12-7.98 (m, 4H), 7.62-7.55 (m, 2H), 7.29 (s, |
| | yl)pyridin-2-yl) | 1H), 3.64 (s, 3H), 1.82 (s, 6H); MS (m/z): |
| | propanenitrile | 471(M + 1)⁺. |
| 6 | 2-(5-(8-(Isoquinolin-4-yl)- | ¹H NMR (300 MHz, DMSO-d₆): δ 9.31 (s, 1H), |
| | 3-methyl-2-oxo-2,3- | 9.13 (s, 1H), 8.91 (s, 1H), 8.34-8.19 (m, 4H), |
| | dihydro-1H-imidazo[4,5- | 8.24 (s, 1H), 7.85-7.791 (m, 5H), 7.06 (s, 1H), |
| | c]quinolin-1-yl)pyridin-2- | 3.65 (s, 3H), 1.46 (s, 6H); MS (m/z): 471 |
| | yl)-2-methylpropanenitrile | (M + 1)⁺. |
| 6a | 8-(Isoquinolin-4-yl)-1-(6- | ¹H NMR (300 MHz, DMSO-d₆): δ 9.34 (s, 1H), |
| | (2-cyanopropan-2- | 9.03 (d, J = 1.8 Hz, 1H), 8.87 (s, 1H), 8.48 (s, |
| | yl)pyridin-3-yl)-3-methyl- | 1H), 8.37 (dd, J = 2.1, 8.4 Hz, 1H), 8.32 (s, 2H), |
| | 2-oxo-2,3-dihydro-1H- | 8.08-8.02 (m, 3H), 7.81 (m, 1H), 7.70 (m, 1H), |
| | imidazo[4,5-c]quinolin-5- | 7.36 (s, 1H), 3.68 (s, 3H), 2.32 (s, 3H), 1.81 (s, |
| | ium methanesulfonate | 6H) |
| 6b | 8-(Isoquinolin-4-yl)-1-(6- | ¹H NMR (300 MHz, DMSO-d₆): δ 9.58 (s, 1H), |
| | (2-cyanopropan-2- | 9.05 (d, J = 2.1 Hz, 1H), 8.99 (d, J = 1.8 Hz, 1H), |
| | yl)pyridin-3-yl)-3-methyl- | 8.62 (m, 2H), 8.51 (m, 1H), 8.40 (dd J = 2.4, 8.4 |
| | 2-oxo-2,3-dihydro-1H- | Hz, 1H), 8.17-8.07 (m, 3H), 7.91 (m, 1H), 7.76 |
| | imidazo[4,5-c]quinolin-5- | (m, 1H), 7.45 (s, 1H), 3.71 (s, 3H), 1.81 (s, 6H); |
| | ium chloride | MS (m/z): 529.2(M + 1)⁺ |
| 7 | 2-(5-(8-(2- | ¹H NMR (300 MHz, DMSO-d₆): δ 11.82 (s, 1H), |
| | Hydroxyquinolin-3-yl)-3- | 9.04 (s, 1H), 8.90 (d, J = 2.1 Hz, 1H), 8.07 (d, J = |
| | methyl-2-oxo-2,3-dihydro- | 9.0 Hz, 1H), 8.00-7.92 (m, 3H), 7.70 (d, J = 7.5 |
| | 1H-imidazo[4,5- | Hz, 1H), 7.52 (m, 3H), 7.34 (d, J = 8.1 Hz, 1H), |
| | c]quinolin-1-yl)pyridin-2- | 7.18 (m, 1H), 3.63 (s, 3H), 1.74 (s, 6H); |
| | yl)-2-methylpropanenitrile | MS (m/z): 487.2(M + 1)⁺. |
| 8 | 2-(5-(8-(6- | ¹H NMR (300 MHz, DMSO-d₆): δ 8.98 (s, 1H), |
| | (Dimethylamino) pyridin- | 8.96 (d, J = 2.1 Hz, 1H), 8.27 (m, 1H), 8.07 (dd, J = |
| | 3-yl)-3-methyl-2-oxo-2,3- | 2.4, 7.2 Hz, 1H), 8.02 (d, J = 8.4 Hz, 1H), 7.88 |
| | dihydro-1H-imidazo[4,5- | (dd, J = 1.5, 9.0 Hz, 1H), 7.60 (m, 1H), 7.50 (d, J = |
| | c]quinolin-1-yl)pyridin-2- | 2.4, 8.71 Hz), 6.94 (s, 1H), 6.62 (d, J = 8.7 Hz, |
| | yl)-2-methylpropanenitrile | 1H), 3.62 (s, 3H), 3.03 (s, 6H), 1.87 (s, 6H); |
| | | MS (m/z): 464.2(M + 1)⁺. |
| 9 | 2-Methyl-2-(5-(3-methyl- | ¹H NMR (300 MHz, DMSO-d₆): δ 9.17 (bs, 1H), |
| | 2-oxo-8-(pyrimidin-5-yl)- | 9.10 (bs, 1H), 8.97 (bs, 1H), 8.81 (s, 1H), 8.30 |
| | 2,3-dihydro-1H- | (d, J = 8.4 Hz, 1H), 8.21 (d, J = 8.7 Hz, 1H), |
| | imidazo[4,5-c]quinolin-1- | 8.07-7.98 (m, 3H), 7.15 (s, 1H), 3.64 (s, 3H), |
| | yl)pyridin-2- | 1.84 (s, 6H); |
| | yl)propanenitrile | MS (m/z): 422.2(M + 1)⁺. |
| 10 | 2-(5-(8-(2,6- | ¹H NMR (300 MHz, DMSO-d₆): δ 9.10 (s, 1H), |
| | Difluoropyridin-3-yl)-3- | 8.92 (d, J = 2.4 Hz, 1H), 8.30-8.16 (m, 3H), |
| | methyl-2-oxo-2,3-dihydro- | 7.94 (d, J = 8.4 Hz, 1H), 7.86 (d, J = 9.0 Hz, 1H), |
| | 1H-imidazo[4,5- | 7.26 (dd, J = 1.8, 8.1 Hz, 1H), 7.18 (s, 1H), 3.64 |
| | c]quinolin-1-yl)pyridin-2- | (s, 3H), 1.83 (s, 6H); MS (m/z): 457.2(M + 1)⁺. |
| | yl)-2-methylpropanenitrile | |
| 11 | 2-(5-(8-(5-Fluoro-2- | ¹H NMR (300 MHz, DMSO-d₆): δ 9.05 (s, 1H), |
| | methoxyphenyl)-3-methyl- | 8.93 (d, J = 2.1 Hz, 1H), 8.26 (m, 1H), 8.10 (d, |
| | 2-oxo-2,3-dihydro-1H- | J = 9.0 Hz, 1H), 7.95 (d, J = 8.4 Hz, 1H), 7.77 |
| | imidazo[4,5-c]quinolin-1- | (dd, J = 2.4, 8.7 Hz, 1H), 7.11 (m, 2H), 7.08 (m, |
| | yl)pyridin-2-yl)-2- | 1H), 6.98 (dd, J = 3.0, 9.3 Hz, 1H), 3.67 (s, 3H), |
| | methylpropanenitrile | 3.63 (s, 3H), 1.77 (s, 6H); |
| | | MS (m/z): 468.2(M + 1)⁺. |
| 12 | 2-(5-(8-(2-Fluoro-5- | ¹H NMR (300 MHz, DMSO-d₆): δ 9.11 (s, 1H), |
| | (trifluoromethyl) phenyl)- | 8.93 (d, J = 2.4 Hz 1H), 8.20 (d, J = 9.0 Hz, 1H), |
| | 3-methyl-2-oxo-2,3- | 7.90-7.87 (m, 2H), 7.86-7.77 (m, 2H), 7.68-7.45 |
| | dihydro-1H-imidazo[4,5- | (m, 1H), 7.18 (s, 2H), 7.23 (bs, 1H), 3.64 (s, |
| | c]quinolin-1-yl)pyridin-2- | 3H), 1.78 (s, 6H); |
| | yl)-2-methylpropanenitrile | MS (m/z): 506.2 (M + 1)⁺. |
| 13 | 2-(5-(8-(2,4- | ¹H NMR (300 MHz, DMSO-d₆): δ 9.05 (s, 1H), |
| | Dimethoxypyrimidin-5-yl)- | 8.92 (d, J = 2.1, 1H), 8.23 (m, 2H), 8.09 (d, J = |
| | 3-methyl-2-oxo-2,3- | 8.7 Hz, 1H), 7.95 (d, J = 8.4 Hz, 1H), 7.78 (dd, J = |
| | dihydro-1H-imidazo[4,5- | 1.8, 9.0 Hz, 1H), 7.17 (d, J = 1.5 Hz, 1H), 3.90 |
| | c]quinolin-1-yl)pyridin-2- | (s, 3H), 3.84 (s, 3H), 3.62 (s, 3H), 1.79 (s, 6H); |
| | yl)-2-methylpropanenitrile | MS (m/z): 482.2 (M + 1)⁺. |

### Example 14

### 2-(5-(3-(Cyanomethyl)-2-oxo-8-(pyridin-3-yl)-2,3-dihydro-1H-imidazo[4,5-c]quinolin-1-yl)pyridin-2-yl)-2-methylpropanenitrile

The title compound was prepared by following the procedure as described for Example 1, except that methyl iodide of step 4 was replaced by 2-bromoacetonitrile. ¹H NMR (300 MHz, DMSO-d₆): δ 9.03 (s, 1H), 8.89 (s, 1H), 8.65 (br s, 2H), 8.32 (d, J=8.7 Hz, 1H), 8.04 (dd, J=1.8, 8.4 Hz, 1H), 7.97-7.90 (m, 2H), 7.66 (d, J=7.5 Hz, 1H), 7.39-7.38 (m, 1H), 7.23 (s, 1H), 5.12 (s, 2H), 1.91 (s, 6H); MS (m/z): 446.2 (M+1)⁺.

### Example 15

### 1-(6-(Dimethylamino)pyridin-3-yl)-3-methyl-8-(pyridin-3-yl)-1H-imidazo[4,5-c]quinolin-2(3H)-one

The title compound was prepared by following the procedure as described for Example 1, except that N²,N²-dimethylpyridine-2,5-diamine (commercially available, 5.5 mmol) was used instead of 2-(5-aminopyridin-2-yl)-2-methylpropanenitrile. ¹H NMR (300 MHz, DMSO-d₆): δ 9.021 (s, 1H), 8.636 (s, 1H), 8.57 (d, J=4.8 Hz, 1H), 8.324 (s, 1H), 8.14 (d, J=8.7 Hz, 1H), 7.95 (d, J=8.7 Hz, 1H), 7.85 (d, J=8.1 Hz, 1H), 7.78 (d, J=9 Hz, 1H), 7.456 (s, 1H), 7.429 (s, 1H), 6.92 (d, J=9.3 Hz, 1H), 3.611 (s, 3H), 3.158 (s, 6H); MS (m/z): 397.2 (M+1)⁺.

### Example 16

### 2-(5-(8-(6-Amino-5-(trifluoromethyl)pyridin-3-yl)-3-(cyanomethyl)-2-oxo-2,3-dihydro-1H-imidazo[4,5-c]quinolin-1-yl)pyridin-2-yl)-2-methylpropanenitrile

The title compound was prepared by following the procedure as described for Example 1, except that methyl iodide of step 4 was replaced by 2-bromoacetonitrile and pyridin-3-ylboronic acid of step 5 was replaced by 5-amino-6-(trifluoromethyl)pyridin-3-ylboronic acid. ¹H NMR (300 MHz, DMSO-d₆): δ 9.17 (s, 1H), 9.05 (d, J=1.5 Hz, 1H), 8.34 (dd, J=2.4, 8.7 Hz, 1H), 8.27 (d, J=1.8 Hz, 1H), 8.14 (d, J=9.0 Hz, 1H), 8.00-7.96 (m, 2H), 7.66 (d, J=1.8 Hz, 1H), 7.07 (d, J=1.5 Hz, 1H), 6.74 (s, 2H), 5.45 (s, 2H), 1.81 (s, 6H); MS (m/z): 529.2 (M+1)⁺.

### Example 17

### 2-(5-(3-(Cyanomethyl)-2-oxo-8-(quinolin-3-yl)-2,3-dihydro-1H-imidazo[4,5-c]quinolin-1-yl)pyridin-2-yl)-2-methylpropanenitrile

The title compound was prepared by following the procedure as described for Example 1, except that methyl iodide of step 4 was replaced by 2-bromoacetonitrile and pyridin-3-ylboronic acid of step 5 was replaced by quinolin-3-ylboronic acid. ¹H NMR (300 MHz, DMSO-d₆): δ 9.24 (s, 1H), 9.05 (s, 1H), 8.83 (s, 1H), 8.40 (m, 2H), 8.27 (d, J=8.7 Hz, 1H), 8.20 (d, J=8.7 Hz, 1H), 8.03-8.01 (m, 3H), 7.79 (m, 1H), 7.67 (m, 1H), 7.28 (s, 1H), 5.49 (s, 2H), 1.82 (s, 6H); MS (m/z): 496.2 (M+1)⁺.

### Example 18

### 2-(5-(3-Allyl-2-oxo-8-(pyridin-3-yl)-2,3-dihydro-1H-imidazo[4,5-c]quinolin-1-yl)pyridin-2-yl)-2-methylpropanenitrile

The title compound was prepared by following the procedure as described for Example 1, except that methyl iodide of step 4 was replaced by allyl bromide. ¹H NMR (300 MHz, DMSO-d₆): δ 8.99 (s, 1H), 8.57 (s, 1H), 8.34 (dd, J=3.9, 8.4 Hz, 1H), 8.22-8.15 (m, 2H), 8.02-7.98 (m, 2H), 7.78 (d, J=8.1 Hz, 1H), 7.53 (dd, J=4.8, 7.8 Hz, 1H), 7.42 (dd, J=4.8, 7.8 Hz, 1H), 7.14 (d, J=1.8 Hz, 1H), 6.09-6.03 (m, 1H), 5.37-5.28 (m, 2H), 4.79 (d, J=5.1 Hz, 2H), 1.84 (s, 6H); MS (m/z): 447.2 (M+1)⁺.

### Example 19

### 8-(6-Amino-5-(trifluoromethyl)pyridin-3-yl)-1-(6-methoxypyridin-3-yl)-3-methyl-1H-imidazo[4,5-c]quinolin-2(3H)-one

The title compound was prepared by following the procedure as described for Example 1, except that 6-methoxypyridin-3-amine (commercially available, 5.5 mmol) was used instead of 2-(5-aminopyridin-2-yl)-2-methylpropanenitrile and pyridin-3-ylboronic acid of step 4 was replaced by 5-amino-6-(trifluoromethyl)pyridin-3-ylboronic acid. ¹H NMR (300 MHz, DMSO-d₆): δ 8.98 (s, 1H), 8.51 (d, J=3 Hz, 1H), 8.38 (s, 1H), 8.10 (m, 2H), 7.96 (m, 1H), 7.60 (d, J=3 Hz, 1H), 7.18 (m, 2H), 6.75 (s, 2H), 3.98 (s, 3H), 3.60 (s, 3H); MS (m/z): 467.2 (M+1)⁺.

### Example 19a

### 8-(6-Ammonio-5-(trifluoromethyl)pyridin-3-yl)-1-(6-methoxypyridin-3-yl)-3-methyl-2-oxo-2,3-dihydro-1H-imidazo[4,5-c]quinolin-5-ium methanesulfonate

The title compound was prepared by following the General method for preparation of mesylate salts as described in method A, using compound of Example 19. ¹H NMR (300 MHz, DMSO-d₆): δ 9.43 (s, 1H), 8.58 (d, J=3 Hz, 1H), 8.41 (s, 1H), 8.37 (d, J=9 Hz, 1H), 8.27 (d, J=9 Hz, 1H), 8.12 (d, J=9 Hz 1H), 7.65 (s, 1H), 7.31 (s, 1H), 7.22 (d, J=9 Hz, 1H), 3.99 (s, 3H), 3.67 (s, 3H), 2.36 (s, 6H).

### Example 20

### 1-(6-Methoxypyridin-3-yl)-3-methyl-8-(quinolin-3-yl)-1H-imidazo[4,5-c]quinolin-2(3H)-one

The title compound was prepared by following the procedure as described for Example 1, except that 6-methoxypyridin-3-amine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.06 (s, 1H), 8.92 (d, J=3 Hz, 1H), 8.56 (d, J=3 Hz, 1H), 8.42 (s, 1H), 8.23 (d, J=9 Hz, 1H), 8.15-8.09 (m, 2H), 8.08 (d, J=9 Hz, 1H), 7.99 (d, J=9 Hz, 1H), 7.83-7.77 (m, 1H), 7.71-7.66 (m, 1H) 7.47 (s, 1H) 7.24 (d, J=9 Hz, 1H), 4.01 (s, 3H), 3.63 (s, 3H); MS (m/z): 434 (M+1)⁺.

### Example 21

### 2-(1-(6-Methoxypyridin-3-yl)-2-oxo-8-(pyridin-3-yl)-1H-imidazo[4,5-c]quinolin-3(2H)-yl)acetonitrile

The title compound was prepared by following the procedure as described for Example 1, except that 6-methoxypyridin-3-amine (commercially available, 5.5 mmol) was used instead of 2-(5-aminopyridin-2-yl)-2-methylpropanenitrile and methyl iodide of step 4 was replaced by 2-bromoacetonitrile. ¹H NMR (300 MHz, DMSO-d₆): δ 9.21 (s, 1H), 8.61-8.58 (m, 3H), 8.22 (d, J=9 Hz, 1H), 8.13 (dd, J=2.7, 8.7 Hz, 1H), 8.02 (dd, J=1.8, 8.7 Hz, 1H), 7.89-7.86 (m, 1H), 7.50 (dd, J=4.8, 7.8 Hz, 1H), 7.31 (d, J=1.5 Hz, 1H), 7.20 (d, J=9 Hz, 1H), 5.45 (s, 2H), 4.00 (s, 3H); MS (m/z): 409 (M+1)⁺.

The compounds of Examples 22-29 were prepared by following the procedure as described for Example 19, using methyl iodide or 2-bromoacetonitrile and an appropriate boronic acid derivative.

| Ex. No. | Nomenclature | NMR/Mass |
|---|---|---|
| 22 | 1-(6-Methoxypyridin-3- | ¹H NMR (300 MHz, DMSO-d₆): δ 9.07 (s, 1H), |
| | yl)-3-methyl-8-(5- | 9.01 (s, 1H), 8.97 (s, 1H), 8.54-8.53 (d, 1H, |
| | (trifluoromethyl) | J = 3 Hz), 8.20-8.17 (d, 1H, J = 9 Hz), 8.12-8.811 (m, |
| | pyridin-3-yl)-1H- | 2H), 8.08-8.05 (m, 2H), 7.38 (s, 1H), 7.15-7.12 (d, |
| | imidazo[4,5-c] quinolin- | J = 9 Hz, 1H), 3.97 (s, 3H), 3.62 (s, 3H), 2.22 (s, 3H); |
| | 2(3H)-one | MS (m/z): 452.2 (M + 1)⁺. |
| 23 | 1-(6-Methoxypyridin-3- | ¹H NMR (300 MHz, DMSO-d₆): δ 9.08 (s, 1H), |
| | yl)-3-methyl-8-(pyridin- | 8.57-8.59 (dd, 2H, J = 1.2, 4.8 Hz), 8.53-8.54 (d, 1H, |
| | 3-yl)-1H-imidazo[4,5-c] | J = 2.7 Hz), 8.16-8.19 (d, 1H, J = 9 Hz), 8.06-8.09 |
| | quinolin-2(3H)-one | (dd, 1H, J = 2.7, 8.7 Hz), 7.96-8.00 (dd, 1H, J = 1.8, 9 |
| | | Hz), 7.85-7.89 (m, 1H), 7.47-7.51 (dd, 1H, J = 4.8, |
| | | 7.8 Hz), 7.32-7.33 (d, 1H, J = 1.8 Hz), 7.16-7.19 (d, |
| | | 1H, J = 8.7 Hz), 4.00 (s, 3H), 3.62 (s, 3H); MS |
| | | (m/z): 384 (M + 1)⁺. |
| 24 | 2-(1-(6-Methoxypyridin- | ¹H NMR (300 MHz, DMSO-d₆): δ 9.21 (s, 1H), |
| | 3-yl)-2-oxo-8-(quinolin- | 9.91-9.92 (d, 1H, J = 2.1 Hz), 8.61-8.62 (d, 1H, J = 2.7 |
| | 3-yl)-1H-imidazo[4,5- | Hz), 8.42-8.43 (d, 1H, J = 2.1 Hz), 8.23-8.2 (d, 1H, |
| | c]quinolin-3(2H)-yl) | J = 8.7 Hz), 8.15-8.20 (m, 2H), 8.06-8.08 (d, 1H, |
| | acetonitrile | J = 8.1 Hz), 7.97-8.00 (d, 1H, J = 7.8 Hz), 7.78-7.83 |
| | | (m, 1H), 7.67-7.72 (m, 1H), 7.45 (s, 1H), 7.22-7.25 |
| | | (d, 1H, J = 9 Hz), 5.46 (s, 2H), 4.01(s, 3H); MS |
| | | (m/z): 459 (M + 1)⁺. |
| 25 | 8-(6- | ¹H NMR (300 MHz, DMSO-d₆): δ 8.95 (s, 1H), |
| | (Dimethylamino)pyridin- | 8.17-8.16 (d, 1H, J = 3 Hz), 8.08-8.04 (m, 3H), 7.87- |
| | 3-yl)-1-(6- | 8.6 (m, 1H), 7.52 (m, 1H), 7.19-7.16 (d, 2H, |
| | methoxypyridin-3-yl)-3- | J = 9 Hz), 6.68-6.65 (d, 1H, J = 9 Hz), 4.01 (s, 3H), 3.60 |
| | methyl-1H-imidazo[4,5- | (s, 3H), 2.91 (s, 6H); MS (m/z): 481.2 (M + 1)⁺. |
| | c] quinolin-2(3H)-one | |
| 26 | 1-(6-Methoxypyridin-3- | ¹H NMR (300 MHz, DMSO-d₆): δ 9.98 (s, 1H), |
| | yl)-3-methyl-8-(6- | 8.10-8.02 (m, 2H), 7.97-7.96 (m, 1H), 7.56-7.55 (d, |
| | (methylamino)-5- | 1H, J = 3 Hz), 7.17-7.12 (m, 3H), 6.80-6.79 (d, 1H, |
| | (trifluoromethyl)pyridin- | J = 3 Hz), 4.00 (s, 3H), 3.60 (s, 3H), 2.91 (s, 3H); MS |
| | 3-yl)-1H-imidazo[4,5-c] | (m/z): 481.2 (M + 1)⁺. |
| | quinolin-2(3H)-one | |
| 27 | 8-(2-Fluoro-5- | ¹H NMR (300 MHz, DMSO-d₆): δ 9.07 (s, 1H), |
| | (trifluoromethyl)phenyl)- | 8.48-8.49 (d, 1H, J = 2.7 Hz), 8.14-8.17 (d, 1H, J = 9 |
| | 1-(6-methoxypyridin-3- | Hz), 8.01-8.05 (dd, 1H, J = 2.7, 9 Hz), 7.80-7.88 (m, |
| | yl)-3-methyl-1H- | 2H), 7.55-7.65 (m, 2H), 7.32 (s, 1H), 7.06-7.09 (d, |
| | imidazo[4,5-c] quinolin- | 1H, J = 8.7 Hz), 3.94 (s, 3H), 3.62 (s, 3H); MS |
| | 2(3H)-one | (m/z): 469 (M + 1) |
| 28 | 1-(6-Methoxypyridin-3- | ¹H NMR (300 MHz, DMSO-d₆): δ 9.06 (s, 1H), |
| | yl)-3-methyl-8-(pyridin- | 8.60-8.62 (d, 2H, J = 6 Hz), 8.53-8.54 (d, 1H, J = 2.4 |
| | 4-yl)-1H-imidazo[4,5-c] | Hz), 8.16-8.19 (d, 1H, J = 8.7 Hz), 8.06-8.09 (dd, 1H, |
| | quinolin-2(3H)-one | J = 2.7, 8.7 Hz), 7.97-8.01 (dd, 1H, J = 1.8, 8.7 Hz), |
| | | 7.38-7.40 (m, 3H), 7.17-7.20 (d, 1H, J = 8.4 Hz), 4.02 |
| | | (s, 3H), 3.62 (s, 3H); MS (m/z): 384 (M + 1⁾⁺ |
| 29 | 8-(5-Fluoro-2- | ¹H NMR (300 MHz, DMSO-d₆): δ 9.01 (s, 1H), |
| | methoxyphenyl)-1-(6- | 8.47-8.48 (d, 1H, J = 2.7 Hz), 8.05-8.08 (d, 1H, J = 9 |
| | methoxypyridin-3-yl)-3- | Hz), 7.99-8.03 (dd, 1H, J = 2.7, 8.7 Hz), 7.72-7.75 |
| | methyl-1H-imidazo[4,5- | (dd, 1H, J = 1.8, 9 Hz), 7.39-7.40 (d, 1H, J = 1.5 Hz), |
| | c]quinolin-2(3H)-one | 7.04-7.17 (m, 4H), 3.95 (s, 3H), 3.64 (s, 6H); MS |
| | | (m/z): 431(M + 1)⁺ |

The compounds of Examples 30-35 were prepared by following the procedure as described for Example 19, using 6-ethoxypyridin-3-amine instead of 6-methoxypyridin-3-amine and an appropriate boronic acid derivative.

| Ex. No. | Nomenclature | NMR/Mass |
|---|---|---|
| 30 | 8-(6-Amino-5- | ¹HNMR (300 MHz, DMSO-d₆): δ 9.00 (s, 1H), 8.10- |
| | (trifluoromethyl) pyridin- | 7.94 (m, 3H), 7.58-7.57 (d, 1H, J = 3 Hz), 7.18-7.17 (d, |
| | 3-yl)-1-(6-ethoxypyridin-3- | 1H, J = 3 Hz), 7.12-7.09 (d, 1H, J = 9 Hz), 6.75 (s, 2H), |
| | yl)-3-methyl-1H- | 4.45-4.38 (q, 2H, J = 9 Hz, J = 6 Hz, J = 6 Hz), 3.61 (s, |
| | imidazo[4,5-c]quinolin- | 3H), 1.41-1.37 (t, 3H, J = 6 Hz, J = 6 Hz); MS |
| | 2(3H)-one | (m/z): 481.2 (M + 1)⁺. |
| 31 | 8-(6-(Dimethylamino) | ¹HNMR (300 MHz, DMSO-d₆): δ 8.97(s, 1H), 8.15- |
| | pyridin-3-yl)-1-(6- | 8.14 (d, J = 3.0 Hz, 1H), 8.08-8.01 (m, 3H), 7.89-7.85 |
| | ethoxypyridin-3-yl)-3- | (dd, J = 3 Hz, J = 9 Hz, 1H), 7.58-7.53 (m, 2H), 7.15- |
| | methyl-1H-imidazo [4,5- | 7.12 (m, 3H), 6.67-6.64 (m, 2H), 4.53-4.43 (m, 2H), |
| | c]quinolin-2(3H)-one | 3.6 (s, 3H), 3.63 (s, 3H), 3.07 (s, 6H), 1.44-1.39 (t, |
| | | 3H, J = 6 Hz); MS (m/z): 441(M + 1)⁺. |
| 32 | 1-(6-Ethoxypyridin-3-yl)- | ¹HNMR (300 MHz, DMSO-d₆): δ 9.10 (s, 1H), 8.94- |
| | 3-methyl-8-(quinolin-3-yl)- | 8.93 (d, J = 3.0 Hz, 1H), 8.54-8.53 (d, 1H, J = 3 Hz), |
| | 1H-imidazo[4,5- | 8.24-8.14 (m, 3H), 8.11-8.08 (m, 2H), 7.98-7.95 (d, |
| | c]quinolin-2(3H)-one | J = 9 Hz, 1H), 7.83-7.77 (t, 1H, J = 9 Hz), 7.71-7.68 (t, |
| | | 1H, J = 9 Hz), 7.44 (s, 1H), 7.20-7.17 (d, J = 9 Hz), 4.48- |
| | | 4.42 (q, 2H, J = 3 Hz, J = 9 Hz), 3.63 (s, 3H), 1.41-1.36 |
| | | (t, 3H, J = 6 Hz); MS (m/z): 448.2 (M + 1)⁺. |
| 33 | 8-(2,6-Difluoropyridin-3- | ¹HNMR (300 MHz, DMSO-d₆): δ 9.07 (s, 1H), 8.44 |
| | yl)-1-(6-ethoxypyridin-3- | (d, J = 2.4 Hz, 1H), 8.27 (m, 1H), 8.26 (d, J = 9 Hz, 1H), |
| | yl)-3-methyl-1H- | 8.01 (dd, J = 9 Hz, 2.7 Hz, 1H), 7.84 (d, J = 9 Hz, 1H), |
| | imidazo[4,5-c]quinolin- | 7.38 (s, 1H), 7.32 (dd, J = 8.1 Hz, 2.7 Hz, 1H), 7.08 (d, |
| | 2(3H)-one | J = 8.7 Hz, 1H), 4.44 (m, 2H), 3.61 (s, 3H), 1.40 (t, |
| | | J = 7.2 Hz, 3H); MS (m/z): 434 (M + 1)⁺. |
| 34 | 1-(6-Ethoxypyridin-3-yl)- | ¹H NMR (300 MHz, DMSO-d₆): δ 9.03 (s, 1H), 8.64 |
| | 8-(2-methoxypyrimidin-5- | (s, 2H), 8.49 (d, J = 1.8 Hz, 1H), 8.16 (d, J = 9 Hz, 1H), |
| | yl)-3-methyl-1H- | 8.05 (dd, J = 8.7 Hz, 1.8 Hz, 1H), 7.96 (dd, J = 8.7 Hz, |
| | imidazo[4,5-c]quinolin- | 1.5 Hz, 1H), 7.24 (d, J = 1.8 Hz, 1H), 7.14 (d, J = 8.7 Hz, |
| | 2(3H)-one | 1H), 4.48 (m, 2H), 3.95 (s, 3H), 3.61 (s, 3H), 1.42 (t, |
| | | J = 7.2 Hz, 3H); MS (m/z): 429(M + 1)⁺. |
| 35 | 1-(6-Ethoxypyridin-3-yl)- | ¹HNMR (300 MHz, DMSO-d₆): δ 9.04 (s, 1H), 8.93 |
| | 3-methyl-8-(quinolin-6-yl)- | (d, J = 2.7 Hz, 1H), 8.54 (d, J = 2.7 Hz, 1H), 8.34 (d, |
| | 1H-imidazo[4,5- | J = 8.4 Hz, 1H), 8.20 (d, J = 8.7 Hz, 1H), 8.02 (m, 4H), |
| | c]quinolin-2(3H)-one | 7.78 (d, J = 9 Hz, 1H), 7.63 (m, 1H), 7.40 (s, 1H), 7.20 |
| | | (d, J = 8.7 Hz, 1H), 4.38 (m, 2H), 3.62 (s, 3H), 1.41 (t, |
| | | J = 6.9 Hz, 3H); MS (m/z): 448(M + 1)⁺. |

The compounds of Examples 36-41 were prepared by following the procedure as described for Example 19, using 6-methoxy-2-methylpyridin-3-amine instead of 6-methoxypyridin-3-amine, methyl iodide or 2-bromoacetonitrile and an appropriate boronic acid derivative.

| Ex. No. | Nomenclature | NMR/Mass |
|---|---|---|
| 36 | 2-(1-(6-Methoxy-2- | ¹HNMR (300 MHz, DMSO-d₆): δ 9.23 (s, 1H), 8.91- |
| | methylpyridin-3-yl)-2- | 7.90 (d, 1H, J = 3 Hz), 8.39 (s, 1H), 8.24-8.20 (m, 2H), |
| | oxo-8-(quinolin-3-yl)- | 8.09-8.06 (m, 2H), 7.99-7.96 (d, 1H, J = 9 Hz), 7.81 |
| | 1H-imidazo[4,5- | (m, 1H), 7.69 (m, 1H), 7.34 (s, 1H), 7.07-7.04 (d, |
| | c]quinolin-3(2H)-yl) | 1H, J = 9 Hz), 5.49 (s, 2H), 3.99 (s, 3H), 2.26 (s, 3H); |
| | acetonitrile | MS (m/z): 473(M + 1)⁺. |
| 37 | 2-(1-(6-Methoxy-2- | ¹HNMR (300 MHz, DMSO-d₆): δ 9.04 (s, 1H), 8.74 |
| | methylpyridin-3-yl)-2- | (s, 1H), 8.35-8.32 (d, 1H, J = 9 Hz), 7.90-7.82 (m, 2H), |
| | oxo-8-(6- | 7.76-7.73 (d, 1H, J = 9 Hz), 7.65-7.62 (d, 1H, J = 9 Hz), |
| | (trifluoromethyl)pyridin- | 7.35 (s, 1H), 6.89-6.86 (d, 1H, J = 9 Hz), 5.19-4.94 (m, |
| | 3-yl)-1H-imidazo[4,5- | 2H), 4.07 (s, 3H), 2.33 (s, 3H); MS (m/z): |
| | c]quinolin-3(2H)-yl) | 491(M + 1)⁺. |
| | acetonitrile | |
| 38 | 8-(6-Amino-5- | ¹H NMR (300 MHz, DMSO-d₆): δ 9.01 (s, 1H), 8.36 |
| | (trifluoromethyl)pyridin- | (s, 1H), 8.11-8.08 (d, 1H, J = 9 Hz), 7.98-7.92 (m, 2H), |
| | 3-yl)-1-(6-methoxy-2- | 7.58 (s, 1H), 7.07 (s, 1H), 6.97-6.94 (d, 1H, J = 9 Hz), |
| | methylpyridin-3-yl)-3- | 6.76 (s, 2H), 3.96 (s, 3H), 3.62 (s, 3H), 2.20 (s, 3H); |
| | methyl-1H- | MS (m/z): 505 (M + 1)⁺. |
| | imidazo[4,5-c]quinolin- | |
| | 2(3H)-one | |
| 39 | 1-(6-Methoxy-2- | ¹H NMR (300 MHz, DMSO-d₆): δ 9.09 (s, 2H), |
| | methylpyridin-3-yl)-3- | 8.21-8.18 (d, 1H, J = 9 Hz), 8.12-8.09 (d, 1H, J = 9 Hz), |
| | methyl-8-(5- | 8.02 (s, 1H), 7.96-7.93 (d, 1H, J = 9 Hz), 7.25 (s, 1H), |
| | (trifluoromethyl)pyridin- | 6.97-6.94 (d, J = 9 Hz, 1H), 3.95 (s, 3H), 3.64 (s, 3H), |
| | 3-yl)-1H-imidazo[4,5- | 2.22 (s, 3H); MS (m/z): 466.2(M + 1)⁺. |
| | c]quinolin-2(3H)-one | |
| 40 | 8-(6-(Dimethylamino) | ¹H NMR (300 MHz, DMSO-d₆): δ 8.97 (s, 1H), |
| | pyridin-3-yl)-1-(6- | 8.13-8.05 (m, 2H), 7.93-7.90 (d, 1H, J = 9 Hz), 7.87- |
| | methoxy-2- | 7.84 (d, J = 9 Hz, 1H), 7.51-7.48 (m, 1H), 7.02-6.96 |
| | methylpyridin-3-yl)-3- | (m, 3H), 6.68-6.65 (d, J = 9 Hz, 1H), 4.00 (s, 3H), 3.61 |
| | methyl-1H- | (s, 3H), 3.05 (s, 6H), 2.21 (s, 3H); |
| | imidazo[4,5-c] | MS (m/z): 441.2 (M + 1)⁺ |
| | quinolin-2(3H)-one | |
| 41 | 1-(6-Methoxy-2- | ¹H NMR (300 MHz, DMSO-d₆): δ 9.08 (s, 1H), 8.89 |
| | methylpyridin-3-yl)-3- | (s, 1H), 8.39 (s, 1H), 8.24-8.21 (d, 1H, J = 9 Hz), 8.16- |
| | methyl-8-(quinolin-3- | 8.13 (d, 1H, J = 9 Hz), 8.08-8.05 (d, 1H, J = 9 Hz), 8.01- |
| | yl)-1H-imidazo[4,5- | 7.96 (m, 2H), 7.83-7.78 (t, 1H, J = 9 Hz), 7.72-7.67 (t, |
| | c]quinolin-2(3H)-one | 1H, J = 9 Hz), 7.35 (s, 1H), 7.06-7.03 (d, 1H, J = 9 Hz), |
| | | 3.98 (s, 3H), 3.98 (s, 3H), 3.74 (s, 3H), 2.21 (s, 3H); |
| | | MS (m/z): 448 (M + 1)⁺. |

### Example 42

### 5-(3-(Cyanomethyl)-2-oxo-8-(pyridin-3-yl)-2,3-dihydro-1H-imidazo[4,5-c]quinolin-1-yl)picolinonitrile

The title compound was prepared by following the procedure as described for Example 1, except that 5-aminopicolinonitrile (commercially available, 5.5 mmol) was used instead of 2-(5-aminopyridin-2-yl)-2-methylpropanenitrile and methyl iodide of step 4 was replaced by 2-bromoacetonitrile. ¹H NMR (300 MHz, DMSO-d₆): δ 9.24 (s, 1H), 9.19 (d, J=3 Hz, 1H), 8.65 (d, J=3 Hz, 1H), 8.58 (dd, J=1.2, 4.5 Hz, 1H), 8.54 (dd, J=2.4, 8.4 Hz, 1H), 8.47 (d, J=8.4 Hz, 1H), 8.25 (d, J=9 Hz, 1H), 8.03 (dd, J=1.8, 9.6 Hz, 1H), 7.90-7.87 (m, 1H), 7.48 (dd, J=4.8, 7.8 Hz, 1H), 7.28 (d, J=1.5 Hz, 1H), 5.48 (s, 2H); MS (m/z): 404 (M+1)⁺.

### Example 43

### 5-(3-(1-Cyanoethyl)-2-oxo-8-(pyridin-3-yl)-2,3-dihydro-1H-imidazo[4,5-c]quinolin-1-yl)picolinonitrile

The title compound was prepared by following the procedure as described for Example 1, except that 5-aminopicolinonitrile (commercially available, 5.5 mmol) was used instead of 2-(5-aminopyridin-2-yl)-2-methylpropanenitrile and methyl iodide of step 4 was replaced by 2-bromopropanenitrile. ¹H NMR (300 MHz, DMSO-d₆): δ 9.18 (s, 1H), 8.68 (s, 1H), 8.63 (d, J=3 Hz, 1H), 8.56-8.52 (m, 1H), 8.47 (d, J=9 Hz, 1H) 8.27 (d, J=9 Hz, 1H), 8.15-8.02 (m, 1H), 7.98 (d, J=3 Hz, 1H), 7.56 (m, 1H), 7.13 (s, 1H), 6.96 (s, 1H), 6.17 (m, 1H), 1.90 (d, J=7.2 Hz, 3H); MS (m/z): 418 (M+1)⁺.

### Example 44

### 5-(3-Methyl-2-oxo-8-(quinolin-3-yl)-2,3-dihydro-1H-imidazo[4,5-c]quinolin-1-yl)picolinonitrile

The title compound was prepared by following the procedure as described for Example 1, except that 5-aminopicolinonitrile (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.01 (d, J=2.1 Hz, 1H), 8.96 (s, 1H), 8.90 (s, 1H), 8.32 (d, J=8.7 Hz, 1H), 8.12-8.22 (m, 3H), 7.98-8.05 (m, 2H), 7.90 (d, J=8.1 Hz, 1H), 7.76-7.81 (m, 1H), 7.63-7.68 (m, 1H), 7.45 (d, J=1.2 Hz, 1H), 3.73 (s, 3H); MS (m/z): 429 (M+1)⁺.

### Example 45

### 5-(8-(6-Amino-5-(trifluoromethyl)pyridin-3-yl)-3-methyl-2-oxo-2,3-dihydro-1H-imidazo[4,5-c]quinolin-1-yl)picolinonitrile

The title compound was prepared by following the procedure as described for Example 1, except that 5-aminopicolinonitrile (commercially available, 5.5 mmol) was used instead of 2-(5-aminopyridin-2-yl)-2-methylpropanenitrile and pyridin-3-ylboronic acid of step 5 was replaced by 6-amino-5-(trifluoromethyl)pyridin-3-ylboronic acid. ¹H NMR (300 MHz, DMSO-d₆): δ 9.14-9.13 (d, 1H, J=3 Hz), 9.04 (s, 1H), 8.49-8.39 (m, 3H), 8.13-8.10 (d, 1H, J=9 Hz), 8.00-7.97 (m, 1H), 7.62 (s, 1H), 7.14 (s, 1H), 6.77 (s, 2H), 3.62 (s, 3H), MS (m/z): 461.9 (M+1)⁺.

The compounds of Examples 46-50 were prepared by following the procedure as described for Example 44, using an appropriate boronic acid derivative.

| Ex. No. | Nomenclature | NMR/Mass |
|---|---|---|
| 46 | 5-(8-(2-Fluoropyridin-3- | ¹HNMR (300 MHz, DMSO-d₆): δ 9.12 (s, 1H), 9.10- |
| | yl)-3-methyl-2-oxo-2,3- | 9.11 (d, 1H, J = 1.8 Hz), 8.39-8.49 (m, 2H), 8.18-8.25 |
| | dihydro-1H-imidazo[4,5- | (m, 2H), 8.06-8.11 (m, 1H), 7.87-7.90 (d, 1H, J = 9 |
| | c]quinolin-1-yl) | Hz), 7.45-7.49 (m, 1H), 7.36 (s, 1H), 3.63 (s, 3H); |
| | picolinonitrile | MS (m/z): 397(M + 1)⁺. |
| 47 | 5-(8-(6-Fluoropyridin-3- | ¹HNMR (300 MHz DMSO-d₆): δ 9.11-9.12 (d, 1H |
| | yl)-3-methyl-2-oxo-2,3- | J = 1.5 Hz), 9.10 (s, 1H), 8.40-8.46 (m, 2H), 8.31 (s, |
| | dihydro-1H-imidazo[4,5- | 1H), 8.18-8.21 (d, 1H, J = 9 Hz), 8.08-8.09 (m, 1H), |
| | c]quinolin-1-yl) | 7.95-7.98 (d, 1H, J = 8.7 Hz), 7.28-7.30 (m, 2H), 3.63 |
| | picolinonitrile | (s, 3H); MS (m/z): 397 (M + 1)⁺. |
| 48 | 5-(8-(6-Methoxypyridin- | ¹HNMR (300 MHz, DMSO-d₆): δ 9.13 (s, 1H), 9.07 |
| | 3-yl)-3-methyl-2-oxo-2,3- | (s, 1H), 8.45 (s, 2H), 8.26 (s, 1H), 8.15-8.18 (d, 1H, |
| | dihydro-1H-imidazo[4,5- | J = 9 Hz), 7.91-7.94 (d, 1H, J = 8.1 Hz), 7.77-7.80 (d, 1H, |
| | c]quinolin-1- | J = 7.8 Hz), 7.21 (s, 1H), 6.89-6.92 (d, 1H, J = 7.8 Hz), |
| | yl)picolinonitrile | 3.89 (s, 3H), 3.63 (s, 3H); MS (m/z): 409 (M + 1)⁺. |
| 49 | 5-(3-Methyl-2-oxo-8- | ¹HNMR (300 MHz, DMSO-d₆): δ 9.06-9.14 (m, 2H), |
| | (pyridin-3-yl)-2,3- | 8.65 (s, 1H), 8.58-8.59 (d, 1H, J = 4.2 Hz), 8.43-8.49 |
| | dihydro-1H-imidazo[4,5- | (m, 2H), 8.19-8.22 (d, 1H, J = 9 Hz), 7.97-8.00 (d, 1H, |
| | c]quinolin-1-yl) | J = 9 Hz), 7.88-7.90 (d, 1H, J = 8.1 Hz), 7.47-7.51 (dd, |
| | picolinonitrile | 1H, J = 4.5, 7.5 Hz), 7.30 (s, 1H), 3.64 (s, 3H); |
| | | MS (m/z): 379 (M + 1)⁺. |
| 50 | 5-(8-(6- | ¹HNMR (300 MHz, DMSO-d₆): δ 9.12-9.09 (d, J = 9 Hz, |
| | (Dimethylamino)pyridin- | 1H), 8.49-8.42 (m, 2H), 8.15-8.13 (d, J = 6 Hz, 2H), |
| | 3-yl)-3-methyl-2-oxo-2,3- | 7.95-7.92 (d, J = 3 Hz, 1H), 7.64-7.51 (m, 2H), 7.16 (s, |
| | dihydro-1H-imidazo[4,5- | 1H), 6.81 (s, 1H), 3.62 (s, 3H), 3.10 (s, 6H); |
| | c]quinolin-1- | MS (m/z): 422 (M + 1)⁺ |
| | yl)picolinonitrile | |

The following compound was prepared by the procedure as described for Example 42, using an appropriate boronic acid derivative.

### Example 51

### 5-(3-(Cyanomethyl)-2-oxo-8-(quinolin-3-yl)-2,3-dihydro-1H-imidazo[4,5-c]quinolin-1-yl)picolinonitrile

¹HNMR (300 MHz, DMSO-d₆): δ 9.25 (s, 1H), 9.22 (d, 1H, J=2.1 Hz), 8.96 (d, 1H, J=2.4

Hz), 8.57 (d, 1H, J=2.4 Hz), 8.52 (s, 1H), 8.46 (d, 1H, J=2.1 Hz), 8.22 (m, 2H), 8.06 (m, 2H), 7.77 (m, 2H), 7.43 (d, 1H, J=1.5 Hz), 5.49 (s, 2H); MS (m/z): 454 (M+1)⁺.

The following compound was prepared by the procedure as described for Example 43, using an appropriate boronic acid derivative.

### Example 52

### 5-(3-(1-Cyanoethyl)-2-oxo-8-(quinolin-3-yl)-2,3-dihydro-1H-imidazo[4,5-c]quinolin-1-yl)picolinonitrile

¹HNMR (300 MHz, DMSO-d₆): δ 9.30 (s, 1H), 9.22-9.23 (d, 1H, J=2.1 Hz), 8.98-8.99 (d, 1H, J=2.4 Hz), 8.60-8.64 (dd, 1H, J=3, 9 Hz), 8.49-8.58 (m, 2H), 8.21-8.33 (m, 2H), 8.01-8.13 (m, 2H), 7.79-7.94 (m, 2H), 7.43-7.44 (d, 1H, J=1.2 Hz), 3.43 (s, 3H);

MS (m/z): 468 (M+1)⁺.

### Example 53

### 3-Methyl-8-(pyridin-3-yl)-1-(6-(trifluoromethyl)pyridin-3-yl)-1H-imidazo[4,5-c]quinolin-2(3H)-one

The title compound was prepared by following the procedure as described for Example 1, except that 6-(trifluoromethyl)pyridin-3-amine (commercially available, 5.5 mmol) was used instead of 2-(5-aminopyridin-2-yl)-2-methylpropanenitrile. ¹H NMR (300 MHz, DMSO-d₆): δ 9.17 (d, J=1.8 Hz, 1H), 9.10 (s, 1H), 8.61 (d, J=1.8 Hz, 1H), 8.57-8.54 (m, 1H), 8.52 (d, J=1.8 Hz, 1H), 8.34 (d, J=8.1 Hz, 1H), 8.21 (d, J=8.7 Hz, 1H), 7.99 (dd, J=2.1, 9.0 Hz, 1H), 7.82-7.79 (m, 1H), 7.42 (dd, J=4.8, 7.89 Hz, 1H), 7.18 (d, J=1.8 Hz, 1H), 3.64 (s, 3H); MS (m/z): 422.1 (M+1)⁺.

### Example 54

### 3-Methyl-8-(quinolin-3-yl)-1-(6-(trifluoromethyl)pyridin-3-yl)-1H-imidazo[4,5-c]quinolin-2(3H)-one

The title compound was prepared by following the procedure as described for Example 1, except that 6-(trifluoromethyl)pyridin-3-amine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.21 (d, J=3 Hz, 1H), 9.12 (s, 1H), 8.97 (d, J=3 Hz, 1H), 8.55-8.56 (m, 1H), 8.39 (s, 1H), 8.36-8.35 (m, 1H), 8.23 (d, J=9 Hz, 1H), 8.17-8.16 (m, 1H), 8.06 (d, J=9 Hz, 1H), 7.95 (d, J=9 Hz, 1H), 7.79-7.80 (m, 1H), 7.69-7.67 (m, 1H), 7.38 (d, J=3 Hz, 1H), 3.66 (s, 3H); MS (m/z): 472 (M+1)⁺.

### Example 55

### 8-(6-Amino-5-(trifluoromethyl)pyridin-3-yl)-3-methyl-1-(6-(trifluoromethyl)pyridin-3-yl)-1H-imidazo[4,5-c]quinolin-2(3H)-one

The title compound was prepared by following the procedure as described for Example 1, except that 6-(trifluoromethyl)pyridin-3-amine (commercially available, 5.5 mmol) was used instead of 2-(5-aminopyridin-2-yl)-2-methylpropanenitrile and pyridin-3-ylboronic acid of step 5 was replaced by 6-amino-5-(trifluoromethyl)pyridin-3-ylboronic acid. ¹H NMR (300 MHz, DMSO-d₆): δ 9.15 (s, 1H), 9.08 (s, 1H), 8.54-8.51 (d, 1H, J=9 Hz), 8.42 (s, 1H), 8.30-8.27 (d, 1H, J=9 Hz), 8.15-8.12 (d, 1H, J=9 Hz), 8.01-7.98 (d, 1H, J=3H), 7.57 (s, 1H), 7.11 (s, 1H), 6.75 (s, 2H), 3.63 (s, 3H); MS (m/z): 505 (M+1)⁺.

### Example 55a

### 8-(6-Ammonio-5-(trifluoromethyl)pyridin-3-yl)-3-methyl-2-oxo-1-(6-(trifluoromethyl)pyridin-3-yl)-2,3-dihydro-1H-imidazo[4,5-c]quinolin-5-ium methanesulfonate

The title compound was prepared by following the General method for preparation of mesylate salts as described in method A. ¹H NMR (300 MHz, DMSO-d₆): δ 9.46 (s, 1H), 9.18 (s, 1H), 8.60 (d, J=9 Hz, 1H), 8.46 (s, 1H), 8.36 (m, 3H), 7.63 (s, 1H), 7.24 (s, 1H), 3.69 (s, 3H), 2.37 (s, 6H).

The compounds of Examples 56 and 57 were prepared by following the procedure as described for Example 53, using the appropriate boronic acid derivative.

| Ex. No. | Nomenclature | NMR/Mass |
|---|---|---|
| 56 | 3-Methyl-1,8-bis(6- | ¹HNMR (300 MHz, DMSO-d₆): δ 9.17-9.14 (m, |
| | (trifluoromethyl)pyridin- | 2H), 8.80-8.79 (d, 1H, J = 3 Hz), 8.51-8.50 (m, |
| | 3-yl)-1H-imidazo [4,5- | 1H), 8.33-8.31 (m, 1H), 8.25-8.22 (d, 1H, |
| | c]quinolin-2(3H)-one | J = 9 Hz), 8.11-8.10 (m, 1H), 8.06-8.02 (dd, 1H, |
| | | J = 3 Hz, J = 9 Hz), 7.93-7.90 (d, 1H, J = 9 Hz), 7.30- |
| | | 7.29 (d, 1H, 3 Hz), 3.65 (s, 1H); |
| | | MS (m/z): 467.2 (M + 1)⁺. |
| 57 | 8-(2,6-Difluoropyridin- | ¹H NMR (300 MHz, DMSO-d₆): δ. 9.12 (s, 1H), |
| | 3-yl)-3-methyl-1-(6- | 8.50 (d, J = 6.9 Hz, 1H), 8.47 (s, 1H), 8.32 (m, |
| | (trifluoromethyl)pyridin- | 3H), 7.88 (d, J = 9 Hz, 1H), 7.29 (m, 2H), 3.64(s, |
| | 3-yl)-1H-imidazo [4,5- | 3H,); MS (m/z): 458 (M + 1)⁺. |
| | c]quinolin-2(3H)-one | |

The compound of Example 58 was prepared by following the procedure as described for Example 53, using 2-chloro-6-(trifluoromethyl)pyridin-3-amine instead of 6-methoxypyridin-3-amine and an appropriate boronic acid derivative.

### Example 58

### 6-Chloro-5-(3-methyl-2-oxo-8-(quinolin-3-yl)-2,3-dihydro-1H-imidazo[4,5-c]quinolin-1-yl)picolinonitrile

¹H NMR (300 MHz, DMSO-d₆): δ 9.14 (s, 1H), 8.93 (s, 1H), 8.85 (d, J=7.8 Hz, 1H), 8.48 (d, J=8.1 Hz, 1H), 8.34 (s, 1H), 8.27 (d, J=8.7 Hz, 1H), 8.17 (d, J=9 Hz, 1H), 8.06 (d, J=8.1 Hz, 1H), 7.95 (d, J=8.1 Hz, 1H), 7.79 (t, J=7.2 Hz, 1H), 7.67 (t, J=7.2 Hz), 3.67 (s, 3H); MS (m/z): 506 (M+1)⁺.

### Example 59

### 8-(6-Amino-5-(trifluoromethyl)pyridin-3-yl)-1-(2-chloro-6-(trifluoromethyl) pyridin-3-yl)-3-methyl-1H-imidazo[4,5-c]quinolin-2(3H)-one

The title compound was prepared by following the procedure as described for Example 1, except that 2-chloro-6-(trifluoromethyl)pyridin-3-amine (commercially available, 5.5 mmol) was used instead of 2-(5-aminopyridin-2-yl)-2-methylpropanenitrile and pyridin-3-ylboronic acid of step 5 was replaced by 6-amino-5-(trifluoromethyl)pyridin-3-ylboronic acid. ¹H NMR (300 MHz, DMSO-d₆): δ 9.07 (s, 1H), 8.79 (d, J=8.1 Hz, 1H), 8.39 (d, J=9.0 Hz, 2H), 8.14 (d, J=9.0 Hz, 1H), 8.00 (d, J=10.5 Hz, 1H), 7.54 (s, 1H), 6.93 (s, 1H), 6.75 (s, 2H), 3.65 (s, 3H); MS (m/z): 539 (M+1)⁺.

### Example 60

### 1-(6-Chloropyridin-3-yl)-3-methyl-8-(pyridin-3-yl)-1H-imidazo[4,5-c]quinolin-2(3H)-one

The title compound was prepared by following the procedure as described for Example 1, except that 6-chloropyridin-3-amine (commercially available, 5.5 mmol) was used instead of 2-(5-aminopyridin-2-yl)-2-methylpropanenitrile. ¹H NMR (300 MHz, DMSO-d₆): δ 9.08 (s, 1H), 8.81 (d, J=2.1 Hz, 1H), 8.62-8.57 (m, 2H), 8.28 (dd, J=3.0, 8.7 Hz, 1H), 8.20 (d, J=8.7 Hz, 1H), 7.98-7.86 (m, 3H), 7.50-7.48 (m, 1H), 7.27 (s, 1H), 3.64 (s, 3H); MS (m/z): 388.1 (M+1)⁺.

### Example 61

### 1-(6-Chloropyridin-3-yl)-3-methyl-8-(quinolin-3-yl)-1H-imidazo[4,5-c]quinolin-2(3H)-one

The title compound was prepared by following the procedure as described for Example 1, except that 6-chloropyridin-3-amine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.09 (s, 1H), 8.97 (d, J=1.8 Hz, 1H), 8.85 (d, J=2.1 Hz, 1H), 8.42 (s, 1H), 8.33 (dd, J=2.4, 8.4 Hz, 1H), 8.24 (d J=9 Hz, 1H), 8.15 (d J=9 Hz, 1H), 8.03-7.97 (m, 2H), 7.94 (m, 1H), 7.83-7.78 (m, 1H), 7.72-7.67 (m, 1H), 7.47 (s, 1H), 3.63 (s, 3H); MS (m/z): 438.1 (M+1)⁺.

### Example 62

### 1-(2,6-Dichloropyridin-3-yl)-3-methyl-8-(pyridin-3-yl)-1H-imidazo[4,5-c]quinolin-2(3H)-one

The title compound was prepared by following the procedure as described for Example 1, except that 2,6-dichloropyridin-3-amine (commercially available, 5.5 mmol) was used instead of 2-(5-aminopyridin-2-yl)-2-methylpropanenitrile. ¹H NMR (300 MHz, DMSO-d₆): δ 9.41 (d, J=1.8 Hz, 1H), 9.10 (s, 1H), 8.77 (d, J=3.6 Hz, 1H), 8.61-8-53 (m, 3H), 8.03 (d, J=9 Hz, 1H), 7.73 (dd, J=2.1, 9 Hz, 1H), 7.75 (dd, J=4.8, 8.1 Hz, 1H), 7.10 (d, J=3.9 Hz, 1H), 3.65 (s, 3H); MS (m/z): 467.9 [M+2Na]⁺.

### Example 63

### 1-(6-Chloro-2-(trifluoromethyl)pyridin-3-yl)-3-methyl-8-(pyridin-3-yl)-1H-imidazo[4,5-c]quinolin-2 (3H)-one

The title compound was prepared by following the procedure as described for Example 1, except that 2-chloro-6-(trifluoromethyl)pyridin-3-amine (commercially available, 5.5 mmol) was used instead of 2-(5-aminopyridin-2-yl)-2-methylpropanenitrile. ¹H NMR (300 MHz, DMSO-d₆): δ 9.13 (s, 1H), 8.79 (d, J=7.8 Hz, 1H), 8.57 (d, J=6 Hz, 2H), 8.43 (d, J=8.1 Hz, 1H), 8.20 (d, J=9 Hz, 1H), 8.00 (dd, J=8, 9 Hz, 1H), 7.79 (d, J=8.1 Hz, 1H), 7.43 (dd, J=4.8, 7.8 Hz, 1H), 7.01 (d, J=0.9 Hz, 1H), 3.67 (s, 3H); MS (m/z): 455.9 [M]⁺.

### Example 64

### 1-(6-(Dimethylamino)pyridin-3-yl)-3-methyl-8-(quinolin-3-yl)-1H-imidazo[4,5-c]quinolin-2(3H)-one

The title compound was prepared by following the procedure as described for Example 1, except that N2,N2-dimethylpyridine-2,5-diamine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.051 (s, 2H), 8.87 (m, 1H), 8.34 (m, 2H), 8.16 (d J=11.1 Hz 2H), 7.98 (d, J=8.4 Hz, 1H), 7.84 (d, J=7.8 Hz, 1H), 7.76 (m, 1H), 7.58 (m, 2H), 6.73 (d, J=9 Hz, 1H), 3.268 (s, 6H), 3.736 (s, 1H); MS (m/z): 447 (M+H)⁺.

### Example 65

### 3-Methyl-8-(quinolin-3-yl)-1-(quinolin-6-yl)-1H-imidazo[4,5-c]quinolin-2 (3H)-one

The title compound was prepared by following the procedure as described for Example 1, except that quinolin-6-amine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹HNMR (300 MHz, DMSO-d₆): δ 9.14 (s, 1H), 9.07 (m, 1H), 8.46-8.43 (m, 2H), 8.19-7.49 (m, 11H), 6.83 (s, 1H), 3.70 (s, 3H);

MS (m/z): 454 (M+1)⁺.

### Example 66

### 3-Methyl-1-(quinolin-6-yl)-8-(5-(trifluoromethyl)pyridin-3-yl)-1H-imidazo[4,5-c]quinolin-2(3H)-one

The title compound was prepared by following the procedure as described for Example 65, except that quinolin-3-ylboronic acid was replaced by 5-(trifluoromethyl)pyridin-3-ylboronic acid. ¹H NMR (300 MHz, DMSO-d₆): δ 9.16 (s, 1H), 9.03 (d, J=2.7 Hz, 1H), 8.87 (s, 1H), 8.56 (s, 1H), 8.41-8.31 (m, 2H), 8.17-7.98 (m, 5H), 7.61 (s, 1H), 7.52 (dd, J=4.2, 8.7 Hz, 1H), 6.76 (s, 1H), 3.69 (s, 3H); MS (m/z): 472 (M+1)⁺.

### Example 67

### 8-(6-Amino-5-(trifluoromethyl)pyridin-3-yl)-3-methyl-1-(quinolin-6-yl)-1H-imidazo[4,5-c]quinolin-2 (3H)-one

The title compound was prepared by following the procedure as described for Example 1, except that quinolin-6-amine (commercially available, 5.5 mmol) was used instead of 2-(5-aminopyridin-2-yl)-2-methylpropanenitrile and pyridin-3-ylboronic acid of step 5 was replaced by replaced by 6-amino-5-(trifluoromethyl)pyridin-3-ylboronic acid. ¹H NMR (300 MHz, DMSO-d₆): δ 9.07 (s, 1H), 9.02 (d, J=7.8 Hz, 1H), 8.37 (d, J=7.8 Hz, 1H), 8.11 (m, 4H), 7.85 (d, J=7.5 Hz, 2H), 7.53 (q, J=4.2 Hz, 1H), 7.26 (s, 1H), 6.70 (s, 2H), 6.57 (s, 1H), 3.67 (s, 3H); MS (m/z): 487 (M+1)⁺.

### Example 68

### 3-Methyl-1-(2-morpholino ethyl)-8-(pyridin-3-yl)-1H-imidazo[4,5-c]quinolin-2(3H)-one

The title compound was prepared by following the procedure as described for Example 1, except that 2-morpholinoethanamine (commercially available, 5.5 mmol) was used instead of 2-(5-aminopyridin-2-yl)-2-methylpropanenitrile. ¹H NMR (300 MHz, DMSO-d₆): δ 9.15 (d, J=2.1 Hz, 1H), 8.94 (s, 1H), 8.65 (dd, J=1.2, 4.5 Hz, 1H), 8.53 (d, J=1.5 Hz, 1H), 8.31 (m, 1H), 8.17 (d, J=9 Hz, 1H), 8.37 (dd, J=1.5, 9 Hz, 1H), 7.58 (dd, J=4.5, 7.8 Hz, 1H), 4.56 (t, J=6.9 Hz, 2H), 3.56 (s, 3H), 3.49 (t, J=4.2 Hz, 4H), 2.72 (t, J=6.6 Hz, 2H), 2.24 (m, 4H); MS (m/z): 390 (M+1)⁺.

### Example 69

### 8-(6-Amino-5-(trifluoromethyl)pyridin-3-yl)-3-methyl-1-(2-morpholinoethyl)-1H-imidazo[4,5-c]quinolin-2(3H)-one

The title compound was prepared by following the procedure as described for Example 1, except that 2-morpholinoethanamine (commercially available, 5.5 mmol) was used instead of 2-(5-aminopyridin-2-yl)-2-methylpropanenitrile and pyridin-3-ylboronic acid of step 5 was replaced by 6-amino-5-(trifluoromethyl)pyridin-3-ylboronic acid. ¹H NMR (300 MHz, DMSO-d₆): δ 8.89 (s, 1H), 8.76 (s, 1H), 8.36 (s, 1H), 8.22 (s, 1H), 8.09 (d, J=9 Hz, 1H), 7.97 (d, J=8.7 Hz, 1H), 6.74 (s, 2H), 4.55 (m, 3H), 3.54 (s, 3H), 3.50 (m, 4H), 2.72 (m, 3H), 2.49 (m, 2H); MS (m/z): 473.2 (M+1)⁺.

### Example 70

### 3-Methyl-1-(2-morpholinoethyl)-8-(quinolin-3-yl)-1H-imidazo[4,5-c]quinolin-2(3H)-one

The title compound was prepared by following the procedure as described for Example 1, except that 2-morpholinoethanamine (commercially available, 5.5 mmol) was used instead of 2-(5-aminopyridin-2-yl)-2-methylpropanenitrile and pyridin-3-ylboronic acid of step 5 was replaced by quinolin-3-ylboronic acid. ¹H NMR (300 MHz, DMSO-d₆): δ 9.50 (d, J=2.1 Hz, 1H), 8.95 (s, 1H), 8.87 (d, J=1.8 Hz, 1H), 8.66 (s, 1H), 8.24-8.08 (m, 4H), 7.84-7.79 (m, 1H), 7.72-7.67 (m, 1H), 4.60-4.58 (m, 2H), 3.57 (s, 3H), 3.46-3.40 (m, 4H), 2.74 (m, 2H), 2.38 (m, 4H); MS (m/z): 440 (M+1).

### Example 71

### 3-Methyl-1-(6-(4-methylpiperazin-1-yl)pyridin-3-yl)-8-(pyridin-3-yl)-1H-imidazo[4,5-c]quinolin-2(3H)-one

Compound of example 60 (0.010 g, 0.026 mmol) and 1-methyl piperazine (1 mL, 9.02 mmol) were subjected to microwave irradiation for 30 minutes at 130° C. Crude product was purified (silica gel column, MeOH/CHCl₃ as eluent) to obtain the title compound. ¹H NMR (300 MHz, DMSO-d₆): δ 10.21 (s, 1H), 9.07 (s, 1H), 8.59 (s, 1H), 8.46 (d, J=2.4 Hz, 1H), 8.18 (d, J=8.7 Hz, 1H), 8.02-7.91 (m, 3H), 7.54-7.49 (m, 1H), 7.41 (d, J=1.5 Hz, 1H), 7.28 (d, J=9 Hz, 1H), 4.61-4.56 (m, 2H), 3.62 (s, 3H), 3.16-3.02 (m, 6H), 2.90 (s, 3H); MS (m/z): 452 (M+1)⁺.

### Example 72

### 1-(6-Chloro-2,4′-bipyridin-3-yl)-3-methyl-8-(pyridin-3-yl)-1H-imidazo[4,5-c]quinolin-2(3H)-one

The title compound was prepared by following the procedure as described for Example 1, except that 6-chloro-2,4′-bipyridin-3-amine (commercially available, 5.5 mmol) was used instead of 2-(5-aminopyridin-2-yl)-2-methylpropanenitrile. ¹HNMR (300 MHz, DMSO-d₆): δ 9.41 (d, J=1.8 Hz, 1H), 9.1 (s, 1H), 8.77 (d, J=3.6 Hz, 1H), 8.61 (m, 2H), 8.03 (d, J=9 Hz, 1H), 7.73 (dd, J=2.1, 9 Hz, 1H), 7.75 (dd, J=4.8, 8.1 Hz, 1H), 7.10 (d, J=3.9 Hz, 1H), 3.65 (s, 3H); MS (m/z): 467.9 [M+2Na]⁺.

### Example 73

### 3-Methyl-1-(6-morpholinopyridin-3-yl)-8-(quinolin-3-yl)-1H-imidazo[4,5-c]quinolin-2(3H)-one

A compound of Example 61 (0.022 g, 0.050 mmol) and morpholine (2 mL) were subjected to microwave irradiation for 20 minutes at 129° C. in microwave vessel. After completion, the reaction was quenched in water and extracted using chloroform. The crude product was further purified by silica gel column chromatography using MeOH/CHCl₃ as eluent to obtain the title compound. ¹H NMR (300 MHz, DMSO-d₆): δ 9.02 (s, 1H), 8.93 (s, 1H), 8.45 (m, 2H), 8.27 (s, 1H), 8.21 (d, J=9 Hz, 1H), 8.09 (d, J=9 Hz, 1H), 7.91 (d, J=9 Hz, 1H), 7.85 (m, 1H), 7.76 (s, 1H), 7.66 (m, 3H) 3.91 (m, 4H), 3.72 (m, 4H), 3.70 (s, 3H); MS m/z 489 (M+1)⁺.

### Example 74

### 8-(6-Amino-5-(trifluoromethyl)pyridin-3-yl)-3-methyl-1-(2-(trifluoromethyl)pyrimidin-5-yl)-1H-imidazo[4,5-c]quinolin-2 (3H)-one

The title compound was prepared by following the procedure as described for Example 1, except that 2-(trifluoromethyl)pyrimidin-5-amine (commercially available, 5.5 mmol) was used instead of 2-(5-aminopyridin-2-yl)-2-methylpropanenitrile and pyridin-3-ylboronic acid of step 5 was replaced by 5-amino-6-(trifluoromethyl)pyridin-3-ylboronic acid. ¹H NMR (300 MHz, DMSO-d₆): δ 9.49 (s, 2H), 9.07 (s, 1H), 8.50 (d, J=5.4 Hz, 1H), 8.16 (d, J=8.7 Hz, 1H), 8.02 (dd, J=8.7, 1.5 Hz, 1H), 7.72 (s, 1H), 7.36 (s, 1H), 6.73 (s, 2H), 3.64 (s, 3H); MS (m/z): 506 (M+1)⁺.

### Example 75

### 8-(5-Amino-6-methoxypyridin-3-yl)-1-(6-methoxypyridin-3-yl)-3-methyl-1H-imidazo[4,5-c]quinolin-2(3H)-one

The title compound was prepared by following the procedure as described for Example 1, except that 6-methoxypyridin-3-amine (commercially available, 5.5 mmol) was used instead of 2-(5-aminopyridin-2-yl)-2-methylpropanenitrile and pyridin-3-ylboronic acid of step 5 was replaced by 5-amino-6-methoxypyridin-3-ylboronic acid. ¹H NMR: (300 Hz, DMSO d₆): δ 8.96 (s, 1H), 8.47-8.46 (d, 1H, J=3 Hz), 8.08-8.00 (m, 2H), 7.74-7.71 (d, 1H, J=9 Hz), 7.32-7.31 (d, 1H, J=3 Hz), 7.19-7.16 (m, 2H), 6.92-6.91 (d, 1H, J=3 Hz), 5.06 (d, 2H), 3.96 (s, 3H), 3.86 (s, 3H), 3.57 (s, 1H); MS (m/z): 429 (m+1).

### Testing of Compounds

The efficacy of the present compounds can be determined by a number of pharmacological assays well known in the art, such as described below. The exemplified pharmacological assays, which follow herein, have been carried out with the compounds of the present invention.

### Example 76

### Protocol for Kinase Assay (PI3Kα)

p110α Radioactive Lipid Kinase Assay

The assay was designed as in the reference, Journal of Biomolecular Screening, 2002, Vol. 7, No. 5, 441-450, the disclosure of which is incorporated herein by reference for the teaching of the assay.

The p110α biochemical assay was performed using a radioactive assay measuring the incorporation of ³²P into the p110α substrate, phosphatidylinsoitol (PI). For the generation of IC₅₀ curves, the reaction was performed in a 96-well MaxiSorp plates. Plates were pre-coated with 4 μg/well of a 1:1 ratio of phosphatidylinositol (PI: Avanti #840042C) and phosphatidylserine (PS: Avanti #840032C) diluted in CHCl₃. Equal amount of p110α (Upstate Millipore) protein was added to each well, containing 25 μL reaction buffer (50 mM MOPSO pH7.0, 100 mM NaCl, 4 mM MgCl₂, 0.1% (w/v) BSA) whereas, for negative control, only reaction buffer was added. Compounds of the present invention dissolved in DMSO were treated at nine-point dose responses (0.3, 1, 3, 6, 10, 30, 60, 100 and 300 nM). Reactions were initiated by the addition of 25 μM ATP solution (Sigma, USA) containing 50 μCi/mL [γ-³²P]-ATP and incubated at RT for 2 hours with gentle shaking. Reactions were finally terminated by the addition of 100 μL of 50 mM EDTA stock solution. Plates were washed 3 times with TBS buffer. The plates were air dried, Microscint 0 (Perkin Elmer) was added to each well and the plates were sealed. The radioactivity incorporated into the immobilized PI substrate was determined with Top Count (Perkin Elmer). Inhibition was calculated using the following equation:

% inhibition=(D_{cpm}−T_{cpm})/(D_{cpm})×100.

T_{cpm}=³²P-cpm in presence of compounds of the present invention
D_{cpm}=³²P-cpm in DMSO control (enzyme control deducted)
IC₅₀ values for compounds of Example 19 and Example 3a are 2.886 nM and 1.368 nM respectively.

### Example 77

### mTOR Inhibition Assay

The compounds of the present invention were tested at ProQinase, Germany. Compound of Example 3a inhibited mTOR enzyme activity with IC₅₀ value of 4.4 nM.

### Example 78

### Protocol for ALK1 and ALK2 Inhibition Assay

The in vitro kinase assays using recombinant human, catalytic domain of ALK1 (ACVRL1) or ALK2 (ACVR1) kinase GST fusion proteins (Invitrogen, USA) were conducted using a time-resolved fluorescence (TR-FRET) format. Kinase reactions were carried out in a 384-well plate format in a final volume of 20 μl. The standard enzyme reaction buffer consisted of 50 mM Tris HCL (pH: 7.4), 1 mM EGTA, 10 mM MgCl₂, 2 mM DTT, 0.01% Tween-20, 20 nM of ALK1/ALK2 kinase enzyme (Invitrogen, USA), 50 nM of peptide substrate (DNA Topoisomerase 2 alpha (Thr 1342)U1 peptide, Perkin Elmer, USA) and 20 μM of ATP. Various concentrations of compound of Example 3a in DMSO (final concentration 2%), was added to give a final concentration of the compound ranging from 20 μM to 20 pM. [20 nM of enzyme and compound in various concentrations were pre incubated for 10 minutes at 23° C. followed by the addition of 50 nM of the peptide substrate]. Reaction was initiated with the addition of 20 μM of ATP. After incubation for 1 hour at 23° C., kinase reaction was stopped with the addition of 5 μl EDTA (final concentration of 10 mM in 20 μl). Eu donor [Eu cryptate-anti-phospho-Topoisomerase 2-alpha (Thr 1342), Perkin Elmer, USA] at a final concentration of 2 nM was added and the mixture was allowed to equilibrate for 1 hour at 23° C. After irradiation of the kinase reaction at 320 or 340 nm, the energy from the Eu donor was transferred to its acceptor which, in turn, generates light at 665 nm. The intensity of the light emission is proportional to the level of the substrate phosphorylation. The IC₅₀ values for compound of Example 3a were determined by a four-parameter sigmoidal curve fit (Sigma plot or Graph pad). IC₅₀ value for compound of Example 3a for ALK-1 is 42 nM and for ALK-2 is 47 nM.

### Example 79

### Protocol for Western Blot Analysis

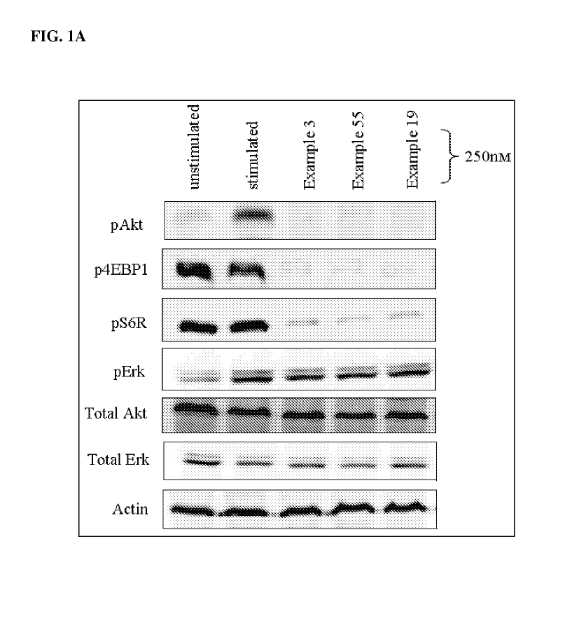

A2780 ovarian cancer cell line (ATCC) were grown to approximately 70% confluence in 100-mm tissue culture dishes and then treated for 1 hour with 50 μL-100 μL compounds of Example 2, 3, 5, 16, 19, 55 and 59. Total protein was extracted using cell lysis buffer (NaCl 200 nM, NP40 0.67%, Tris-Cl, pH 7.5, 67 mM) containing protease inhibitors and phosphatase inhibitors (Beta-glycerol phosphate 40 mM, DTT 1 mM, NaF 0.4 mM, Sodium-Orthovanadate 0.4 mM) at 4° C. for 1 hour. Cell lysates were then centrifuged at 2×10⁴ g for 10 minutes at 4° C., and the protein concentration of the supernatant was quantified using the Bradford's method (BioRad, USA). For SDS-PAGE, 50 μg protein was loaded in SDS-PAGE, transferred to a polyvinylidene difluoride membrane (Bio-Rad, USA) and blocked with 5 mL buffer [5% skim milk and 0.1% Tween] for 1 hour 30 minutes. Membrane were probed with primary antibody (all primary antibodies were from cell signaling with 1:1000 in TBST solution) of respective protein at 4° C. overnight. Peroxidase-labeled anti-rabbit or anti-mouse antibodies (Santacruze, USA) were used as the secondary antibody. Actin and respective whole protein levels were used as the control for protein loading. Protein antigens were detected using Chemiluminescent Substrate (Thermo scientific, USA) and exposed on Kodak station. The results (FIG. 1A-1E) demonstrate that compounds of Example 2, 3, 5, 16, 19, 55, 59 and Example 3a inhibit Akt, S6 and 4EBP1 phosphorylation and hence are inhibitors of PI3K/mTOR pathway.

### Example 80

### Cytotoxicity Assay

Propidium Iodide Assay

The assay was designed as in the reference, Anticancer Drugs, 2002, 13, 1-8, the disclosure of which is incorporated herein by reference for the teaching of the assay.

Cells from cell lines (ATCC) as mentioned in the table given below were seeded at a density of 3000 cells/well in a white opaque 96-well plate. Following incubation at 37° C./5% CO₂ for a period of 18-24 hours, the cells were treated with various concentrations (stock solution was prepared in DMSO and subsequent dilutions were made in media as per ATCC guidelines) of the compounds of the present invention for a period of 48 hours. At the end of treatment, the culture medium was discarded, the cells were washed with 1×PBS and 200 μL of 7 mg/mL propidium iodide was added to each well. The plates were frozen at 70° C. overnight. For analysis, the plates were warmed to RT, allowed to thaw and were read in PoleStar fluorimeter with the fluorescence setting. The percentage of viable cells in the non-treated set of wells was considered to be 100 and the percentage viability following treatment was calculated accordingly. IC₅₀ values were calculated from graphs plotted using these percentages. IC₅₀ values for certain compounds of present invention are depicted in Table 1 and % Inhibition of certain compounds of present invention are depicted in Table 2. The Cell Lines as used in the above assay are:

| Type of Cancer | Abbreviation | Cell Line | Abbreviation |
|---|---|---|---|
| Ovarian | C1 | A2780 | C1a |
| Prostate | C2 | PC3 | C2a |
| Breast | C3 | MDA MB 231 | C3a |
| | | MDA MB 468 | C3b |
| | | BT 549 | C3c |
| | | MCF7 | C3d |
| Pancreatic | C4 | PANC 1 | C4a |
| | | AsPC 1 | C4b |
| | | BxPC3 | C4c |
| Glioblastoma | C5 | LN229 | C5a |
| | | LN18 | C5b |
| | | U 87 MG | C5c |
| Chronic Myeloid | C6 | K562 | C6a |
| Leukemia (CML) | | T315I | C6b |
| | | KU812/SR | C6c |
| | | KU812 | C6d |
| | | KCL22/SR | C6e |
| | | KCL22 | C6f |

TABLE 1
IC₅₀ Values in μM

| | | Compds. of Example No. | | | | | | | |
|---|---|---|---|---|---|---|---|---|---|
| Cell Lines | | 1 | 2 | 3 | 5 | 14 | 16 | 17 | 19 |
| C1 | C1a | 0.4 | 0.024 | <0.005 | 0.08 | 0.4 | 0.013 | 0.065 | <0.01 |
| C2 | C2a | 0.22 | 0.068 | 0.005 | 0.38 | 0.42 | 0.025 | 0.17 | 0.006 |
| C3 | C3a | — | 0.07 | 0.014 | — | — | 0.07 | 0.17 | 0.17 |
| | C3b | — | 0.3 | 0.06 | — | — | 0.013 | 0.45 | 0.18 |
| | C3c | — | 0.9 | 0.03 | — | — | 0.015 | 0.37 | 0.23 |
| | C3d | — | 0.09 | 0.026 | — | — | 0.03 | 0.25 | 0.054 |
| C4 | C4a | — | 0.1 | 0.01 | — | — | 0.017 | 0.23 | 0.026 |
| | C4b | — | 0.09 | 0.019 | — | — | 0.038 | 0.24 | 0.1 |
| | C4c | — | 0.04 | 0.01 | — | — | 0.008 | 0.085 | 0.016 |
| C5 | C5a | — | 0.55 | 0.056 | — | — | 0.65 | — | 0.096 |
| | C5b | — | 0.33 | 0.144 | — | — | 0.44 | — | 0.096 |
| | C5c | — | — | — | — | — | 7.752 | — | 3.88 |
| C6 | C6a | 0.9 | — | 0.056 | — | — | 0.4 | — | 0.056 |
| | C6b | 3.3 | — | 0.32 | — | — | 2.13 | — | 0.049 |
| | C6c | 0.96 | — | 0.19 | — | — | 0.19 | — | 0.06 |
| | C6d | — | — | — | — | — | — | — | — |
| | C6e | 0.62 | — | 0.06 | — | — | 0.23 | — | 0.06 |
| | C6f | 1.18 | — | 2 | — | — | — | — | — |
| | | Cmpds. of Example No. | | | | | | | |
| Cell Lines | | 20 | 38 | 49 | 53 | 54 | 55 | 59 | 60 |
| C1 | C1a | 0.07 | <0.005 | 0.009 | 0.55 | 0.16 | 0.009 | <0.01 | 0.68 |
| C2 | C2a | 0.27 | 0.0078 | 0.017 | 0.75 | 0.55 | 1.6 | 0.018 | 0.85 |
| C3 | C3a | 0.37 | — | 0.016 | — | 0.55 | 0.035 | <0.01 | — |
| | C3b | 0.47 | — | 0.065 | — | 1.2 | 0.075 | 0.07 | — |
| | C3c | 1 | — | 0.3 | — | 1 | 0.17 | 0.04 | — |
| | C3d | 0.55 | — | 0.03 | — | 0.8 | 0.06 | 0.013 | — |
| C4 | C4a | 0.25 | — | 0.035 | — | 0.95 | — | 0.017 | — |
| | C4b | 0.4 | — | 0.025 | — | 0.55 | 0.04 | 0.042 | — |
| | C4c | 0.45 | — | 0.04 | — | 0.78 | 0.027 | <0.01 | — |
| C5 | C5a | — | — | — | — | — | 0.096 | — | — |
| | C5b | — | — | — | — | — | 0.282 | — | — |
| | C5c | — | — | — | — | — | 7.92 | — | — |
| C6 | C6a | 0.1 | — | — | — | — | 0.056 | — | — |
| | C6b | 1.96 | — | — | — | — | 0.19 | — | — |
| | C6c | 5.44 | — | — | — | — | 0.23 | — | — |
| | C6d | 1.99 | — | — | — | — | | — | — |
| | C6e | 1.09 | — | — | — | — | 0.06 | — | — |
| | C6f | 2.86 | — | — | — | — | | — | — |

Cmpds. of Example No.

| Cell Lines | | 62 | 63 | 67 | 68 | 69 | 70 | 72 |
|---|---|---|---|---|---|---|---|---|
| C1 | C1a | — | — | 0.006 | 1.4 | 0.022 | 0.3 | — |
| C2 | C2a | — | — | 0.015 | 2.4 | 0.07 | 0.9 | — |
| C3 | C3a | — | — | 0.012 | 2.3 | — | 0.7 | — |
| | C3b | — | — | 0.05 | 7 | — | 1.1 | — |
| | C3c | 1.5 | 5.6 | 0.011 | >10 | — | 2.1 | — |
| | C3d | — | 4 | 0.02 | 3 | — | 0.7 | — |
| C4 | C4a | — | — | 0.025 | 4.6 | — | 1 | — |
| | C4b | — | — | 0.035 | 4.6 | — | 1.6 | — |
| | C4c | — | — | 0.0085 | 2.4 | — | 0.7 | — |
| C5 | C5a | 1.032 | — | — | — | — | — | 0.44 |
| | C5b | 1.08 | — | — | — | — | — | 0.992 |
| | C5c | 9.92 | — | — | — | — | — | — |
| C6 | C6a | 0.144 | 0.224 | — | — | — | 0.7 | 0.0144 |
| | C6b | — | — | — | — | — | 2.56 | — |
| | C6c | 0.84 | — | — | — | — | 3.38 | 1.14 |
| | C6d | — | — | — | — | — | 4 | — |
| | C6e | 0.62 | — | — | — | — | 1.57 | 0.23 |
| | C6f | — | — | — | — | — | 2.17 | — |

TABLE 2
% Inhibition at 1 μM

| | | Cmpds. of Example No. | | | | | | | | | |
|---|---|---|---|---|---|---|---|---|---|---|---|
| Cell Lines | | 4 | 6 | 21 | 22 | 24 | 25 | 30 | 37 | 39 | 41 |
| C1 | C1a | 89.18 | 85.27 | 38 | 83.06 | 66 | 83.42 | 94.01 | 70 | 83.42 | 79.99 |
| C2 | C2a | 72.57 | 69.51 | 27 | 67.79 | 39 | 69 | 70.95 | 49.48 | 68.81 | 68 |

| | | Cmpds. of Example No. | | | | | | | | | | | |
|---|---|---|---|---|---|---|---|---|---|---|---|---|---|
| Cell Lines | | 42 | 43 | 45 | 46 | 47 | 48 | 51 | 52 | 56 | 58 | 62 | 63 |
| C1 | C1a | 60-80 | 8 | 52 | 34 | 40-60 at | 40-60 at | 73 | 36 | 57 | 91 | 69 | 88 |
| | | | | | | 10 μM | 10 μM | | | | | | |
| C2 | C2a | 60-80 | 13 | 37 | 27 | 40-60 at | 40-60 at | 41 | 12 | 32 | 60 | 28 | 53 |
| | | | | | | 10 μM | 10 μM | | | | | | |

The symbol — indicates that the compounds were not tested.

### Example 81

### Protocol for Tube Formation Assay

Cell Culture: Human umbilical vein endothelial cells (HUVECs) (ATCC) used in passages 2-7. The cells were grown in endothelial medium (Promocell, Germany) supplemented with 20% fetal bovine serum (FBS), 100 units/mL penicillin, 100 μg/mL streptomycin, 3 ng/mL basic fibroblast growth factor, and 5 units/mL heparin at 37° C. under a humidified 95% (v/v) mixture of air and CO₂.

Tube Formation Assay: 250 μl of growth factor-reduced Matrigel (BD Biosciences) was pipetted into a 24 well tissue culture plate and polymerized for 30 minutes at 37° C. HUVECs incubated in endothelial media containing 1% FBS for 6 hours were harvested after trypsin treatment and suspended in endothelial media containing 1% FBS. Compounds of present invention (75 nM) were added to the cells for 30 minutes at RT before seeding and plated onto the layer of Matrigel at a density of 2×10⁴ cells/well, and followed by the addition of 2 mL of 40 ng/mL VEGF. After 18 hours, the cultures were photographed.

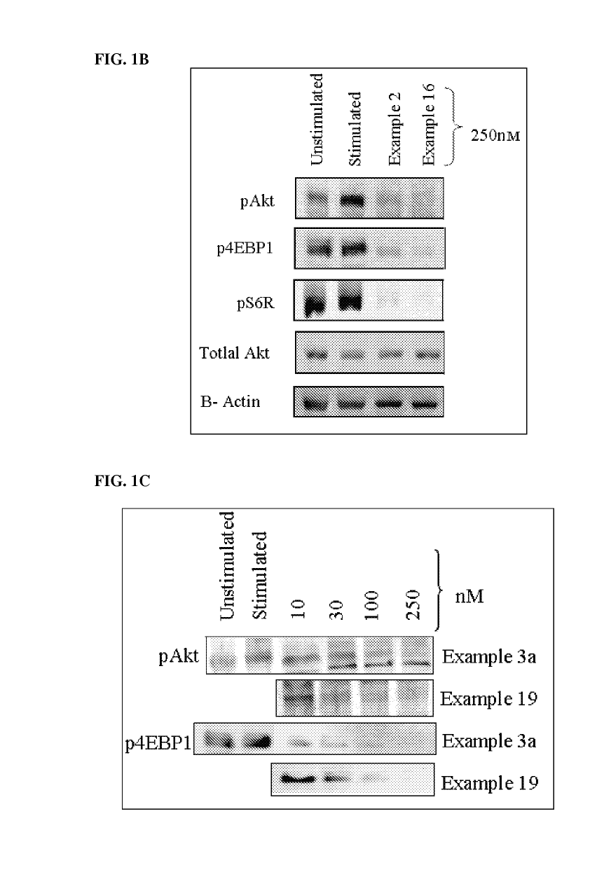

Results: When HUVECs were placed on growth factor-reduced Matrigel in the presence of VEGF, VEGF led to the formation of elongated and robust tube-like structures, which were organized by much larger number of cells compared with the control. FIG. 2 demonstrates that compound of Example 3a effectively abrogated the width and the length of endothelial tubes induced by VEGF.

### Example 82

### Protocol for In-Vivo Assay

Animals: Severe combined immunodeficient (SCID) mice (Male and Female) 6 to 8 weeks old, were used. Animals were housed in suitable cages under specified pathogen-free conditions in rooms maintained at 23° C. and 50% humidity, with a 12-hour light/12-hour dark cycle. The mice were quarantined during the acclimatization period of at least a week.

### Tumor Growth Inhibition Studies In Vivo

Prostate cancer xenograft model: PC3 (human prostate cancer) cell line (ATCC) was maintained in RPMI 1640 (Gibco BRL, Pasley, UK) supplemented with 10% (v/v) FBS. The cells were incubated at 37° C. in a humidified atmosphere containing 5% CO₂. Cells were passaged using trypsin/EDTA for cell detachment once every 3 days. On the day of tumor cell injection, cells were detached from the flasks with trypsin/EDTA, washed once in medium and re-suspended in serum free RPMI 1640 at 5 million cells/0.2 mL volume, and placed on ice. Severe combined immunodeficient mice were injected with 0.2 mL of the cell suspension subcutaneously on the right flank and observed daily for tumor appearance.

Procedure: Tumor-bearing mice were randomized (n=7 per group) in four groups when the mean tumor volume was ˜100 mm³. Each group was closely matched before treatment, which began 2 to 3 weeks after cell transplantation. Twice a week, each xenograft was measured in two dimensions (a=length; b=width) with a caliper. Tumor volume (V) was determined by the following equation: V=ab²/2

Tumor volumes were converted into tumor weights assuming a tumor density of 1 mm³=1 mg. Tumor growth inhibition (TGI) for each group was calculated according to the following formula:

(1−[T−T₀]/[C−C₀])×100

Wherein, T and T₀ are the mean tumor volumes on a specific experimental day and on day 1 of treatment, respectively, for the experimental groups. Likewise, for the control groups, C and C₀ are the mean tumor volumes on a given day and on day 1 of the study, respectively. Animals in each group were observed every day for signs of health deterioration and animal weight was recorded daily out to day 28 post tumor transplantation.

Treatment of animals: Tumor bearing animals were randomized in four groups,
i) Group 1: Control group-Tumor-bearing mice were administered with vehicle
ii) Group 2: Tumor-bearing mice were administered once daily p.o with 3 mg/kg of compound of Example 3a
iii) Group 3: Tumor-bearing mice were administered twice daily (BID) p.o with 3 mg/kg of compound of Example 3a
iv) Group 4: Tumor-bearing mice were administered once daily p.o with 3 mg/kg of the compound of Example 19 using 1 mL tuberculin syringes fitted with feeding needles with round tip and Luer lock hub.

Compounds of the present invention were formulated in 0.5% carboxymethyl cellulose and 0.1% Tween 80 in water. The application volume was 10 mL/kg. Treatment continued for 15 days.

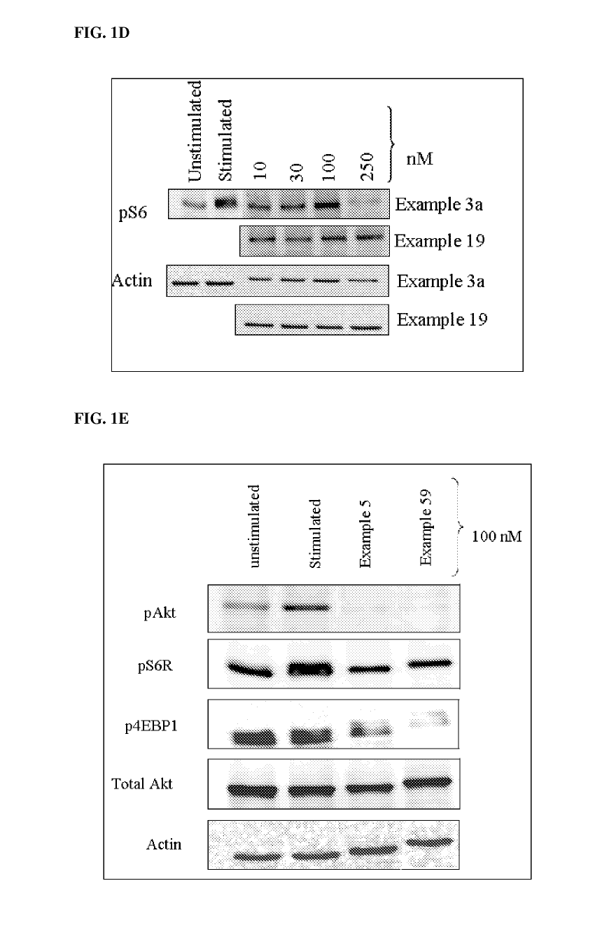

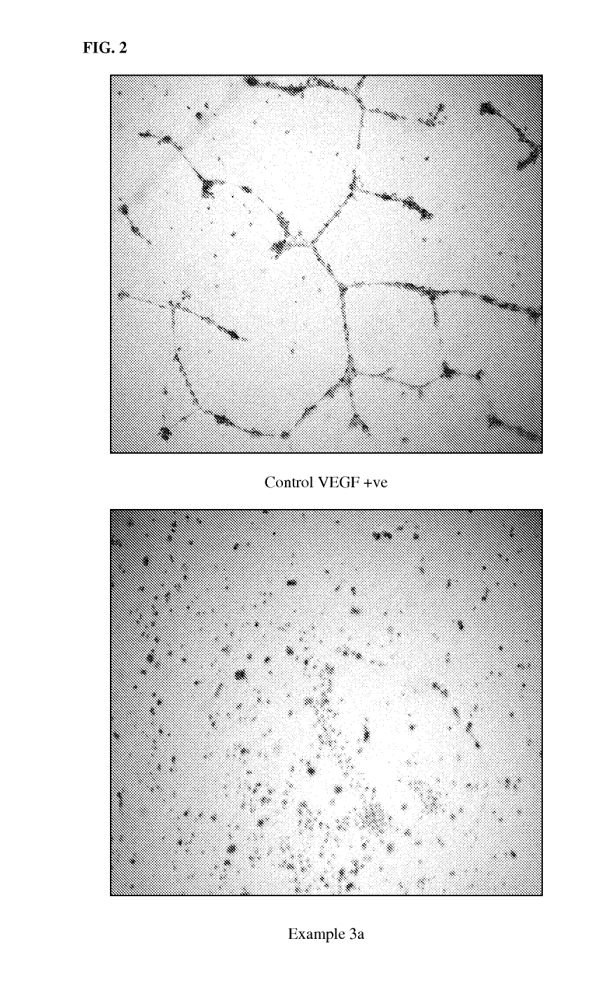

Results: FIG. 3A shows that the compound of Example 19 and the compound of Example 3a effectively inhibit tumor growth in-vivo at a concentration of 3 mpk.

Pancreatic Cancer xenograft model: The human pancreatic carcinoma cells, (PANC-1) (ATCC) were grown in Eagle's Minimum Essential Medium (SAFC, US) supplemented with 10% (v/v) fetal bovine serum. The cells were incubated at 37° C. in a humidified atmosphere containing 5% CO₂. On the day of tumor cell injection, cells were harvested and resuspended in serum free Eagle's Minimum Essential Medium and BD Matrigel™ (BD Biosciences, USA) basement membrane matrix (50:50, v/v) at 5 million cells/0.2 mL volume, and placed on ice. Severe combined immunodeficient mice were injected with 0.2 mL of the cell suspension subcutaneously on the right flank and observed daily for tumor appearance.

Tumor-bearing mice were randomized in two groups when the mean tumor weight was ˜100 mg. Each group was closely matched before treatment, which began one week after cell transplantation. Twice a week, each xenograft was measured in two dimensions (a=length; b=width) with a caliper. Tumor volume (V) was determined by the following equation:

V=ab²/2

Tumor volumes were converted into tumor weights assuming a tumor density of 1 mm³=1 mg. Tumor growth inhibition (TGI) for each group was calculated according to the following formula:

(1−[T−T₀]/[C−C₀])×100

Wherein, T and T₀ are the mean tumor volumes on a specific experimental day and on day 1 of treatment, respectively, for the experimental groups. Likewise, for the control groups, C and C₀ are the mean tumor volumes on a given day and on day 1 of the study, respectively. Animals in each group were observed every day for signs of health deterioration and animal weight was recorded daily.

Treatment of animals: Tumor bearing animals were randomized in two groups,
i) Group 1: Control group-Tumor-bearing mice were administered with vehicle
ii) Group 2: Tumor-bearing mice were administered once daily p.o with 3 mg/kg of compound of Example 3a using 1 mL tuberculin syringes fitted with feeding needles with round tip and Luer lock hub.

Compounds of the present invention were formulated in 0.5% carboxymethyl cellulose and 0.1% Tween 80 in water. The application volume was 10 mL/kg. Treatment continued for 14 days.

Results: FIG. 3B shows that compound of Example 3a effectively inhibited the growth of pancreatic tumors in a mouse xenograft model at the concentration of 3 mpk.

### Example 83

### Con-A-Induced IFN-γ Production from hPBMC

Peripheral blood was collected from normal healthy volunteers after informed consent. Peripheral blood mononuclear cells (hPBMC) were harvested using Ficoll-Hypaque density gradient centrifugation (1.077 g/mL; Sigma Aldrich). hPBMCs were resuspended in RPMI 1640 culture medium (Gibco BRL, Pasley, UK) containing 10% FCS, 100 U/mL penicillin (Sigma Chemical Co. St Louis, Mo.) and 100 mg/mL streptomycin (Sigma Chemical Co. St Louis, Mo.) at 1×10⁶ cells/mL. 1×10⁵ hPBMCs/well were pre-treated with 0.025 μM of compounds of present invention or 0.5% DMSO (vehicle control) for 30 minutes at 37° C. Subsequently, these cells were stimulated with 1 μg/mL concanavalin A (Sigma Chemical Co., St. Louis, Mo.). Following 18 hours of incubation at 37° C., supernatants were collected and stored at −70° C. until assayed for human IFN-γ by ELISA as described by the manufacturer (OptiEIA ELISA sets, BD BioSciences). In every experiment, cyclosporin (1 μM) was used as a positive control for inhibiting induced IFN-γ production. In all experiments, the toxicity of test compounds was ascertained, in parallel, using the MTS (3-(4,5-dimethylthiazol-2-yl)-5-(3-carboxymethoxyphenyl)-2-(4-sulfonyl)-2H-tetrazolium) assay as described in Am. J. Physiol. Cell Physiol., 2003, 285, C813-C822.

| | % Inhibition of IFN-γ | % Toxicity |
|---|---|---|
| Example No. | at 0.025 μM | at 0.025 μM |
| 2 | 58 | 12 |
| 3 | 98 | 21 |
| 16 | 31 | 3 |
| 19 | 93 | 18 |
| 57 | 88 | 14 |

Compounds of the present invention inhibit the proliferation of human T-cells.

### Example 84

### Anti-CD3 mAb and Anti-CD28 mAb-Induced Cytokine Production Assay

Preparation of anti-CD3/anti-CD28 coated plates: 96 well plates were coated with goat anti-mouse IgG, Fc (Millipore) at a concentration of 16.5 μg/mL in coating buffer (8.4 g/mL NaHCO₃, 3.56 g Na₂CO₃, pH 9.5). Following overnight incubation at 4° C., the plates were washed and then incubated with anti-CD3 (3.5 μg/mL; R&D Systems) and anti-CD28 (35 ng/mL; R&D Systems) cocktail for 3 hours. Subsequently, the plates were washed, and used for hPBMC stimulation.

hPBMC stimulation: Peripheral blood was collected from normal healthy volunteers after informed consent. Peripheral blood mononuclear cells (hPBMC) were harvested using Ficoll-Hypaque density gradient centrifugation (1.077 g/mL; Sigma Aldrich). hPBMCs were resuspended in RPMI 1640 culture medium (Gibco BRL, Pasley, UK) containing 10% FCS, 100 U/mL penicillin (Sigma Chemical Co. St Louis, Mo.) and 100 mg/mL streptomycin (Sigma Chemical Co. St Louis, Mo.) at 1.25×10⁶ cells/mL of assay medium. 2.5×10⁵ hPBMCs were added per well of 96-well plate coated with or without anti-CD3/anti-CD28 mAbs. Simultaneously, 0.025 μM of compounds of the present invention or 0.5% DMSO (vehicle control) were added to appropriate wells. The cells were then incubated for 18 hours at 37° C., 5% CO₂ following which supernatants were collected, stored at 70° C. and assayed later for TNF-α, IL-6 and IFN-γ by ELISA (OptiEIA ELISA sets; BD Biosciences). In every experiment, each condition was run in triplicate wells. In all experiments, the toxicity of test compounds was ascertained, in parallel, using the MTS (3-(4,5-dimethylthiazol-2-yl)-5-(3-carboxymethoxyphenyl)-2-(4-sulfonyl)-2H-tetrazolium) assay as described in Am. J. Physiol. Cell Physiol., 2003, 285, C813-C822.

| | % Inhibition of cytokine at 0.025 μM | | | % Toxicity |
|---|---|---|---|---|
| Compound | TNF-α | IL-6 | IFN-γ | at 0.025 μM |
| 2 | 63 | 43 | 71 | 12 |
| 3 | 92 | 84 | 93 | 24 |
| 16 | 81 | 75 | 80 | 0 |
| 19 | 87 | 86 | 87 | 19 |
| 57 | 80 | 94 | 88 | 14 |

Compounds of the present invention inhibit the activation of human T-cells and consequent production of pro-inflammatory cytokines.

### Example 85

### Protocol for Collagen-Induced Arthritis

Induction of Collagen-Induced Arthritis and Treatment with Compound of Example 19

All animal experiments were carried out in accordance with the guidelines of Committee for the Purpose of Control and Supervision of Experiments on Animals (CPCSEA). All animal experiments were approved by Institutional Animal Ethics Committee (IAEC) of Piramal Life Sciences Limited, Mumbai, India. Collagen-induced arthritis was induced in DBA/1 J mice as described in J. Exp. Med., 1985, 162, 637-46. Inbred male DBA/1 J mice (8-10 weeks of age, Jackson Laboratories, Bar Harbor, Me.) were immunized intradermally at the base of the tail with 200 μg type II collagen emulsified in Freund's Complete Adjuvant (FCA) on day 0. On day 17, immunized mice were randomized into different groups based on their body weight. From day 17 onwards, (i) one group of mice started receiving administration of compound of Example 19 (1 mg/kg, p.o., twice daily), (ii) a second group of mice started receiving administration of vehicle (0.5% CMC, p.o., twice daily), and (iii) a third group of mice started receiving administration of Enbrel (3 mg/kg, s.c., once daily). On day 21, all mice were boosted with 200 μg type II collagen emulsified in FCA. The mice (8 per treatment group) were monitored daily (from day 17 onwards) for the development and severity of arthritis using articular index and paw thickness as parameters. Articular index scoring was performed employing the following criteria—Fore limbs (Scale 0-3): 0, no redness or swelling; 1, redness but no swelling; 2, redness and swelling of the paw; 3, redness and severe swelling of the paw. Hind limbs (Scale 0-4): 0, no redness or swelling; 1, redness and mild swelling of paw; 2, redness and moderate swelling of paw and/or swelling of at least one of the digits; 3, redness and moderate/severe swelling of paw, swelling of ankle joint and/or swelling of one or more digits; 4, redness and severe swelling of paw, digits and ankle joint, with joint stiffness and altered angle of digits. The total articular index for a mouse is sum of individual articular index scores of fore limbs and hind limbs. Swelling of each of the paws of mice was measured with constant-tension, spring-loaded calipers (Mitutoyo, Aurora, Ill.). All measurements and scoring were performed by operators blinded to the treatment groups.

Treatment continued daily until day 36 of the study, and the body weight of the animal along with the severity of inflammation for all 4 paws was monitored daily. In every experiment, a group of non-immunized mice was maintained alongside as naïve control. On the last day of experiment, one hour after compound of Example 19, vehicle, or Enbrel administration, the animals were humanely euthanized.

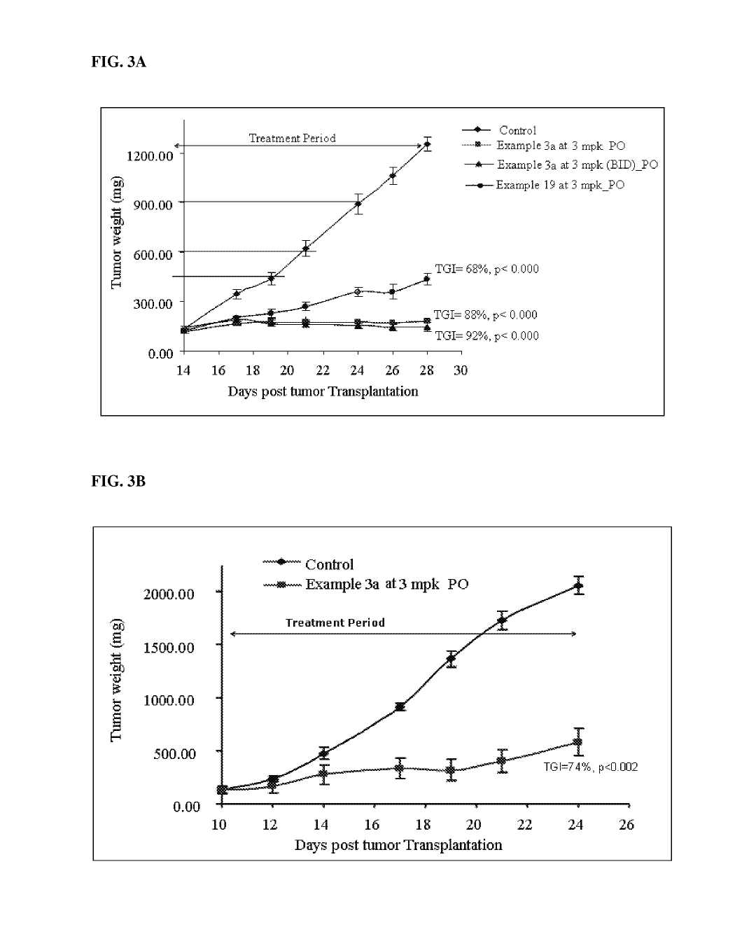

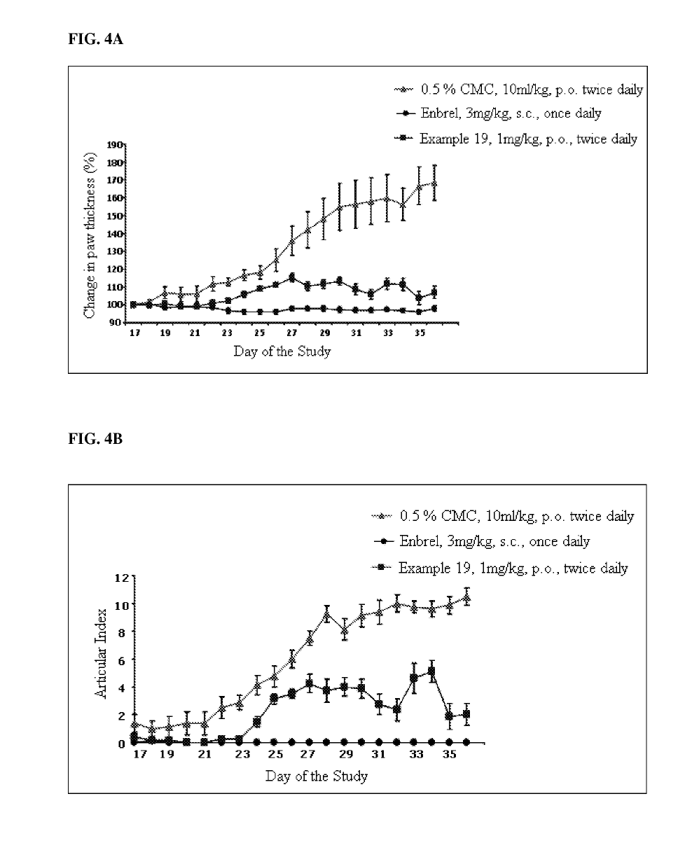

The results as depicted in FIGS. 4a and 4b show that the compound of Example 19 (i) inhibits disease-associated increase in articular index and paw thickness, (ii) distinctly protects against bone erosion and joint space narrowing, and (iii) prominently diminishes joint destruction, hyperproliferative pannus formation and infiltration of inflammatory cells.

It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A compound of formula (I) 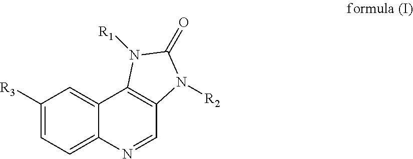
wherein,
R₁ is selected from C₁-C₄ alkylheterocyclyl, C₁-C₄ alkylheteroaryl or heteroaryl, wherein each of heterocyclyl and heteroaryl is optionally substituted with one or more groups selected from R₁₁;
R₂ is C₁-C₄ alkyl, optionally substituted with one or more groups independently selected from —CN or C₂-C₄ alkenyl;
R₃ is selected from heteroaryl or C₆-C₁₄ aryl, wherein each of aryl and heteroaryl is optionally substituted with one or more groups selected from R₃₁;
R₁₁ at each occurrence is independently selected from halo —CN, —ORₓ, —NRₓR_{y}, —NRₓCOR_{y}, —COORₓ, —CONRₓR_{y}, halo-C₁-C₄ alkyl, C₁-C₄ heterocyclyl or heteroaryl, wherein each of alkyl, heterocyclyl, or heteroaryl is optionally substituted with one or more groups independently selected from —CN or C₁-C₄ alkyl;
R₃₁ at each occurrence is independently selected from halogen, —ORₓ, —CN, —NRₓR_{y}, —NRₓCOR_{y}, —COORₓ, —CONRₓR_{y}, halo-C₁-C₄ alkyl or C₁-C₄ alkyl;
wherein Rₓ and R_{y} at each occurrence are independently selected from hydrogen or C₁-C₄ alkyl; or
a stereoisomer, a tautomer, a polymorph, an N-oxide, or a pharmaceutically acceptable salt thereof.

2. The compound according to claim 1, wherein is selected from pyridyl, pyrimidinyl quinolinyl, wherein pyridyl, pyrimidinyl and quinolinyl are optionally substituted with one or more groups independently selected from halogen, —CN, —ORₓ, —NRₓR_{y}, halo-C₁-C₄ alkyl, C₁-C₄ alkyl, heterocyclyl or heteroaryl, wherein each of C₁-C₄ heterocyclyl and heteroaryl is optionally substituted with one or more groups independently selected from —CN or C₁-C₄ alkyl and Rₓ and R_{y} at each occurrence are independently selected from hydrogen or C₁-C₄ alkyl or a stereoisomer, a tautomer, a polymorph, an N-oxide, or a pharmaceutically acceptable salt thereof.

3. The compound according to claim 2, wherein R₁ is a substituted pyridyl group represented by the structural formula 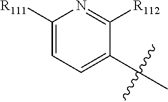
wherein, the symbol [symbol] indicates the point of attachment to the rest of the molecule; R₁₁₁ is selected from —Cl, —CN, —OCH₃, —OC₂H₅, —N(CH₃)₂, —CF₃, —C(CH₃)₂CN, morpholinyl or piperazinylmethyl; and R₁₁₂ is selected front hydrogen, Cl, CH₃ or pyridyl or a stereoisomer, a tautomer, a polymorph, an N-oxide, or a pharmaceutically acceptable salt thereof.

4. The compound according to claim 1, wherein R₂ is methyl, optionally substituted with one or more groups independently selected from —CN or C₂-C₄ alkenyl or a stereoisomer, a tautomer, a polymorph, an N-oxide, or a pharmaceutically acceptable salt thereof.

5. The compound according to claim 4, wherein R₂ is methyl or a stereoisomer, a tautomer, a polymorph, an N-oxide, or a pharmaceutically acceptable salt thereof.

6. The compound according to claim 1, wherein R₃ is heteroaryl optionally substituted with one or more groups independently selected from halogen, —ORₓ, —NRₓR_{y}, C₁-C₄ alkyl or halo-C₁-C₄ wherein Rₓ and R_{y} at each occurrence are independently selected from hydrogen or C₁-C₄ alkyl or a stereoisomer, a tautomer, a polymorph, an N-oxide, or a pharmaceutically acceptable salt thereof.

7. The compound according to claim 6, wherein R₃ is selected from pyridyl or quinolinyl; wherein pyridyl and quinolinyl are optionally substituted with one or more groups independently selected front halogen, —ORₓ, NRₓR_{y}, C₁-C₄ alkyl or halo-C₁-C₄ alkyl, wherein Rₓ and R_{y} at each occurrence are independently selected from hydrogen or C₁-C₄ alkyl or a stereoisomer, a tautomer, a polymorph, an N-oxide, or a pharmaceutically acceptable salt thereof.

8. The compound according to claim 1, wherein R₃ is substituted pyridyl represented by the structural formula 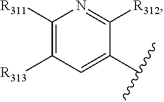
wherein the symbol [symbol] indicates the point of attachment to the rest of the molecule; and each of R₃₁₁, R₃₁₂ and R₃₁₃ is independently selected from hydrogen, halogen, —ORₓ, —NRₓR_{y}, C₁-C₄ alkyl or halo-C₁-C₄ alkyl, wherein Rₓ and R_{y} at each occurrence are independently selected from hydrogen or C₁-C₄ alkyl or a stereoisomer, a tautomer, a polymorph, an N-oxide, or a pharmaceutically acceptable salt thereof.

9. The compound according to claim 8, wherein each of R₃₁₁, R₃₁₂ and R₃₁₃ is independently selected from hydrogen, halogen, —O—C₁-C₄ alkyl, —NH₂, —NH—C₁-C₄ alkyl, —N(C₁-C₄ alkyl), or methyl; wherein methyl is optionally substituted with one to three halogen atoms or a stereoisomer, a tautomer, a polymorph, an N-oxide, or a pharmaceutically acceptable salt thereof.

10. The compound according to claim 9, wherein each of R₃₁₁, R₃₁₂ and R₃₁₃ is independently selected from hydrogen, F, —OCH₃, —NH₂, —NH—CH₃, —N(CH₃)₂ or —CF₃ or a stereoisomer, a tautomer, a polymorph, an N-oxide, or a pharmaceutically acceptable salt thereof.

11. The compound according to claim 9, wherein R₃₁₁ is —NH₂; R₃₁₂ and R₃₁₃ are independently selected from hydrogen, halogen, —O—C₁-C₄ alkyl, —NH₂, —NH—C₁-C₄ alkyl, —N(C₁-C₄ alkyl)₂ or methyl; wherein methyl is optionally substituted with one to three halogen atoms or a stereoisomer, a tautomer, a polymorph, an N-oxide, or a pharmaceutically acceptable salt thereof.

12. The compound according to claim 9, wherein R₃₁₃ is —CF₃ and R₃₁₁ and R₃₁₂ are independently selected from hydrogen, halogen, —O—C₁-C₄ alkyl, —NH₂, —NH—C₁-C₄ alkyl, —N(C₁-C₄ alkyl)₂ or methyl; wherein methyl is optionally substituted with one to three halogen atoms or a stereoisomer, a tautomer, a polymorph, an N-oxide, or a pharmaceutically acceptable salt thereof.

13. The compound according to claim 8, wherein R₃₁₁ is —NH₂ and R₃₁₃ is —CF₃ and R₃₁₂ is selected from hydrogen, halogen, —O—C₁-C₄ alkyl, —NH₂, —NH—C₁-C₄ alkyl, —N(C₁-C₄ alkyl)₂ or methyl; wherein methyl optionally substituted with one to three halogen atoms or a stereoisomer, a tautomer, a polymorph, an N-oxide, or a pharmaceutically acceptable salt thereof.

14. The compound according to claim 13, wherein R₃₁₁ is —NH₂, R₁₁₃ is —CF₃ and R₃₁₂ is hydrogen or a stereoisomer, a tautomer, a polymorph, an N-oxide, or a pharmaceutically acceptable salt thereof.

15. The compound according to claim 1, selected from:
2-Methyl-2-(5-(3-methyl-2-oxo-8-(pyridin-3-yl)-2,3-dihydro-1H-imidazo[4,5-c]quinolin-1-yl)pyridin-2-yl)propanenitrile,
2-Methyl-2-(5-(3-methyl-2-oxo-8-(quinolin-3-yl)-2,3-di hydro-1H-imidazo[4,5-c]1-yl)pyridin-2-yl)propanenitrile,
2-(5-(8-(6-Amino-5-(trifluoromethyl)pyridin-3-yl)-3-methyl-2-oxo-2,3-dihydro-1H-imidazo[4,5-c]quinolin-1-yl)pyridin-2-O-2-methylpropanenitrile,
2-Methyl-2-(5-(3-methyl-2-oxo-8-(5-(trifluoromethyl)pyridin-3-yl)-2,3-dihydro-1H-imidazo[4,5-c]quinolin-1-yl)pyridin-2-yl)propanenitrile,
2-Methyl-2-(5-(3-methyl-2-oxo-8-(quinolin-6-yl)-2,3-dihydro-1H-imidazo[4,5-c]quinolin-1-yl)pyridin-2-yl)propanenitrile,
2-(5-(8-Isoquinolin-4-yl)-3-methyl-2-oxo-2,3-dihydro-1H-imidazo[4,5-c]quinolin-1-yl) pyridin-2-yl)-2-methylpropanenitrile,
2-(5-(8-(2-Hydroxyquinolin-3-yl)-3-methyl-2-oxo-2,3-dihydro-1H-imidazo[4,5-c]quinolin-1-yl)pyridin-2-yl)-2-methylpropanenitrile,
2-(5-(8-(6-(Dimethylamino) pyridin-3-yl)-3-methyl-2-oxo-2,3-dihydro-1H-imidazo[4,5-c]quinolin-1-yl)pyridin-2-yl)-2-methylpropanenitrile,
2-Methyl-2-(5-(3-methyl-2-oxo-8-(pyrimidin-5-yl)-2,3-dihydro-1H-imidazo[4,5-c]quinolin-1-yl)pyridin-2-yl)propanenitrile,
2-(5-(8-(2,6-Difluoropyridin-3-yl)-3-methyl-2-oxo-2,3-dihydro-1H-imidazo[4,5-c]quinolin-1-yl)pyridin-2-yl)-2-methylpropanenitrile,
2-(5-(8-(5-Fluoro-2-methoxyphenyl)-3-methyl-2-oxo-2,3-dihydro-1H-imidazo[4,5-c]quinolin-1-yl)pyridin-2-yl)-2-methylpropanenitrile,
2-(5-(8-(2-Fluoro-5-(trifluoromethyl)phenyl)-3-methyl-2-oxo-2,3-dihydro-1H-imidazo[4,5-c]quinolin-1-yl)pyridin-2-yl)-2-methylpropanenitrile,
2-(5-(8-(2,4-Dimethoxypyrimidin-5-yl)-3-methyl-2-oxo-2,3-dihydro-1H-imidazo[4,5-c]quinolin-1-yl)pyridin-2-yl)-2-methylpropanenitrile,
2-(5-(3-(Cyanomethyl)-2-oxa-8-(pyridin-3-yl)-2,3-dihydro-1H-imidazo[4,5-e]quinolin-1-yl)pyridin-2-yl)-2-methylpropanenitrile,
1-(6-(Dimethylamino)pyridin-3-yl)-3-methyl-8-(pyridin-3-yl)-1H-imidazo[4,5-c]quinolin-2(3H)-one,
2-(5-(8-(6-Amino-5-(trifluoromethyl)pyridin-3-yl)-3-(cyanomethyl)-2-oxo-2,3-dihydro-1H-imidazo[4,5-c]quinolin-1-yl)pyridin-2-yl)-2-methylpropanenitrile,
2-(5-(3-(Cyanomethyl)-2-oxo-8-(quinolin-3-O-2,3-dihydro-1H-imidazo[4,5-c]quinolin-1-yl)pyridin-2-yl)-2-methylpropanenitrile,
2-(5-(3-Allyl-2-oxo-8-(pyridin-3-yl)-2,3-dihydro-1H-imidazo[4,5-c]quinolin-1-yl)pyridin-2-yl)-2-methylpropanenitrile,
8-(6-Amino-5-(trifluoromethyl)pyridin-3-yl)-1-(6-methoxypyridin-3-yl)-3-methyl-1H-imidazo[4,5-c]quinolin-2(3H)-one,
1-(6-Methoxypyridin-3-yl)-3-methyl-8-(quinolin-3-yl)-1H-imidazo[4,5-c]quinolin-2(3H)-one,
2-(1-(6-Methoxypyridin-3-yl)-2-oxa-8-(pyridin-3-yl)-1H-imidazo[4,5-c]quinolin-3(2H)-yl)acetonitrile,
1-(6-Methoxypyridin-3-yl)-3-methyl-8-(5-(trifluoromethyl)pyridin-3-yl)-1H-imidazo[4,5-c]quinolin-2(3H)-one,
1-(6-Methoxypyridin-3-yl)-3-methyl-8-(pyridin-3-yl)-1H-imidazo[4,5-e]quinolin-2(3H)-one,
1-(6-Methoxypyridin-3-yl)-2-oxo-8-(quinolin-3-yl)-1H-imidazo[4,5-c]quinolin-3(2H)-yl) acetonitrile,
8-(6-(Dimethylamino)pyridin-3-yl)-1-(6-methoxypyridin-3-yl)-3-methyl-1H-imidazo[4,5-c]quinolin-2(3H)-one,
1-(6-Methoxypyridin-3-yl)-3-methyl-8-(6-(methylamino)-5-(trifluormethyl)pyridin-3-yl)-1H-imidazo[4,5-c]quinolin-2(3H)-one,
8-(2-Fluoro-5-(trifluoromethyl)phenyl)-1-(6-methoxypyridin-3-yl)-3-methyl-1H-imidazo[4,5-c]quinolin-2(3H)-one,
1-(6-Methoxypyridin-3-yl)-3-methyl-8-(pyridin-4-yl)-1H-imidazo[4,5-c]quinolin-2(3H)-one,
8-(5-Fluoro-2-methoxyphenyl)-1-(6-methoxypyridin-3-yl)-3-methyl-1H-imidazo[4,5-c]quinolin-2(3H)-one,
8-(6-Amino-5-(trifluoromethyl)pyridin-3-yl)-1-(6-ethoxypyridin-3-yl)-3-methyl-1H-imidazo[4,5-c]quinolin-2(3H)-one,
8-(6-(Dimethylamino) pyridin-3-yl)-6-ethoxypyridin-3-yl)-3-methyl-1H-imidazo[4,5-c]quinolin-2(3H)-one,
1-(6-Ethoxypyridin-3-yl)-3-methyl-8-(quinolin-3-yl)-1H-imidazo[4,5-c]quinolin-2(3H)-one,
8-(2,6-Difluoropyridin-3-yl)-1-(6-ethoxypyridin-3-yl)-3-methyl-1H-imidazo[4,5-c]quinolin-2(3H)-one,
1-(6-Ethoxypyridin-3-yl)-8-(2-methoxypyrimidin-5-yl-1-methyl-1H-imidazo[4,5-c]quinolin-2(3H)-one,
1-(6-Ethoxypyridin-3-yl)-3-methyl-8-(quinolin-6-yl)-1H-imidazo[4,5-c]quinolin-2(3H)-one,
2-(1-(6-Methoxy-2-methylpyridin-3-yl)-2-oxo-8-(quinolin-3-yl)-1H-imidazo[4,5-c]quinolin-3(2H)-yl)acetonitrile,
2-(1-(6-Methoxy-2-methylpyridin-3-yl)-2-oxo-8-(6-(trifluoromethyl)pyridin-3-yl)-1H-imidazo[4,5-c]quinolin-3(2H)-yl)acetonitrile,
8-(6-Amino-5-(trifluoromethyl)pyridin-3-yl)-1-(6-methoxy-2-methylpyridin-3-yl)-3-methyl-1H-imidazo[4,5-c]quinolin-2(3H)-one,
1-(6-Methoxy-2-methylpyridin-3-yl)-3-methyl-8-(5-(trifluoromethyl)pyridin-3-yl)-1H-imidazo[4,5-c]quinolin-2 (3H)-one,
8-(6-(Dimethylamino)pyridin-3-yl)-1-(6-methoxy-2-methylpyridin-3-yl)-3-methyl-1H-imidazo[4,5-c]quinolin-2(3H)-one,
1-(6-Methoxy-2-methylpyridin-3-yl)-3-methyl-8-(quinolin-3-yl)-1H-imidazo[4,5-c]quinolin-2(3H)-one,
5-(3-(Cyanomethyl)-2-oxo-8-(pyridin-3-yl)-2,3-dihydro-1H-imidazo quinolin-1-yl)picolinonitrile,
5-(3-(1-Cyanoethyl)-2-oxo-8-(pyridin-3-yl)-2,3-dihydro-1H-imidazo[4,5-c]quinolin-1-yl)picolinonitrile,
5-(3-Methyl-2-oxo-8-(quinolin-3-yl)-2,3-dihydro-1H-imidazo[4,5-c]quinolin-1-yl)picolinonitrile,
5-(8-(6-Amino-5-(trifluoromethyl)pyridin-3-yl)-3-methyl-2-oxo-2,3-dihydro-1H-imidazo[4,5-c]quinolin-1-yl)picolinonitrile,
5-(8-(2-Fluoropyridin-3-yl)-3-methyl-2-oxo-2,3-dihydro-1H-imidazo[4,5-c]quinolin-1-yl)picolinonitrile,
5-(8-(6-Fluoropyridin-3-yl)-3-methyl-2-oxo-2,3-dihydro-1H-imidazo[4,5-c]quinolin-1-yl)picolinonitrile,
5-(8-(6-Methoxypyridin-3-yl)-3-methyl-2-oxo-2,3-dihydro-1H-imidazo[4,5-c]quinolin-1-yl)picolinonitrile,
5-(3-Methyl-2-oxo-8-(pyri 1H-imidazo[4,5-c]quino-1-yl)picolinonitrile,
5-(8-(6-(Dimethylamino)pyridin-3-yl)-3-methyl-2-oxo-2,3-dihydro-1H-imidazo[4,5-c]quinolin-1-yl)picolinonitrile,
5-(3-(Cyanomethyl)-2-oxo-8-(quinolin-3-yl)-2,3-dihydro-1H-imidazo[4,5-c]quinolin-1-yl)picolinonitrile,
5-(3-(1-Cyanoethyl)-2-oxo-8-(quinolin-3-yl)-2,3-dihydro-1H-imidazo[4,5-c]quinolin-1-yl)picolinonitrile;
3-Methyl-8-(pyridin-3-yl)-1-(6-(trifluoromethyl)pyridin-3-yl)-1H-imidazo[4,5-c]quinolin-2(3H)-one,
3-Methyl-8-(quinolin-3-yl)-1-(6-(trifluoromethyl)pyridin-3-yl)-1H-imidazo[4,5-c]quinolin-2(3H)-one,
8-(6-Amino-5-(trifluoromethyl)pyridin-3-yl)-3-methyl-1-(6-(trifluoromethyl)pyridin-3-yl)-1H-imidazo[4,5-c]quinolin-2(3H)-one,
3-Methyl-1,8-bis(6-(trifluoromethyl)pyridin-3-yl)-1H-imidazo[4,5-c]quinolin-2 (3H)-one,
8-(2,6-Difluoropyridin-3-yl)-3-methyl-1-(6-(trifluoromethyl)pyridin-3-yl)-1H-imidazo[4,5-c]quinolin-2(3H)-one,
6-Chloro-5-(3-methyl-2-oxo-8-(quinolin-3-yl)-2,3-dihydro-1H-imidazo[4,5-c]quinolin-1-yl)picolinonitrile,
8-(6-Amino-5-(trifluoromethyl)pyridin-3-yl)-1-(2-chloro-6-(trifluoromethyl)pyridin-3-yl)-3-methyl-1H-imidazo[4,5-c]quinolin-2(3H)-one,
1-(6-Chloropyridin-3-yl)-3-methyl-8-(pyridin-3-yl)-1H-imidazo[4,5-c]quinolin-2(3H)-one,
1-(6-Chloropyridin-3-yl)-3-methyl-8-(quinolin-3-yl)-1H-imidazo[4,5-c]quinolin-2(3H)-one,
1-(2,6-Dichloropyridin-3-yl)-3-methyl-8-(pyridin-3-imidazo[4,5-c]quinolin-2(3H)-one,
1-(6-Chloro-2-(trifluoromethyl)pyridin-3-yl)-3-methyl-8-(pyridin-3-yl)-1H-imidazo[4,5-c]quinolin-2(3H)-one,
1-(6-(Dimethylamino)pyridin-3-yl)-3-methyl-8-(quinolin-3-yl)-1H-imidazo[4,5-c]quinolin-2(3H)-one,
3-Methyl-8-(quinolin-3-yl)-1-(quinolin-6-yl)-1H-imidazo[4,5-c]quinolin-2(3H)-one,
3-Methyl-1-(quinolin-6-yl)-8-(5-(trifluoromethyl)pyridin-3-yl)-1H-imidazo[4,5-c]quinolin-2(3H)-one,
8-(6-Amino-5-(trifluoromethyl)pyridin-3-yl)-3-methyl-1-(quinolin-6-yl)-1H-imidazo[4,5-c]quinolin-2(3H)-one,
3-Methyl-1-(2-morpholinoethyl)-8-(pyridin-3-yl)-1H-imidazo[4,5-e]qui whit-2(3H)-one,
8-(6-Amino-5-(trifluoromethyl)pyridin-3-yl)-3-methyl-1-(2-morpholinoethyl)-1H-imidazo[4,5-c]quinolin-2(3H)-one,
3-Methyl-1-(2-morpholinoethyl)-8-(quinolin-3-yl)-1H-imidazo[4,5-c]quinolin-2(3H)-one,
3-Methyl-1-(6-(4-methylpiperazin-1-yl)pyridin-3-yl)-8-(pyridin-3-yl)-1H-imidazo[4,5-c]quinolin-2(3H)-one,
1-(6-Chloro-2,4′-bipyridin-3-yl)-3-methyl-8-(pyridin-3-yl)-1H-imidazo[4,5-c]quinolin-2(3H)-one,
3-Methyl-1-(6-morpholinopyridin-3-yl)-8-(quinolin-3-yl)-1H-imidazo[4,5-c]quinolin-2(3H)-one,
8-(6-Amino-5-(trifluoromethyl)pyridin-3-yl)-3-methyl-1-(2-(trifluoromethyl)pyrimidin-5-yl)-1H-imidazo[4,5-c]quinolin-2(3H)-one, or
8-(5-Amino-6-methoxypyridin-3-yl)-1-(6-methoxypyridin-3-O-3-methyl-1H-imidazo[4,5-c]quinolin-2(3H)-one;
or a pharmaceutically acceptable salt, a stereoisomer, a tautomer or N-oxide thereof.

16. The compound according to claim 15, selected from:
8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium methanesulfonate,
8-(6-Amino-5-(trifluoromethyl)pyridin-3-yl)-1-(6-(2-cyanopropan-2-yl)pyridin-3-yl)-3-methyl-2-oxo-2,3-dihydro-1H-imidazo[4,5-c]quinolin-5-ium chloride,
8-(Isoquinolin-4-yl)-1-(6-(2-cyanopropan-2-yl)pyridin-3-yl)-3-methyl-2-oxo-2,3-dihydro-1H-imidazo[4,5-c]quinolin-5-ium methanesulfonate,
8-(Isoquinolin-4-yl)-1-(6-(2-cyanopropan-2-yl)pyridin-3-yl)-3-methyl-2-oxo-2,3-dihydro-1H-imidazo[4,5-c]quinolin-5-ium chloride,
8-(6-Ammonio-5-(trifluoromethyl)pyridin-3-yl)-1-(6-methoxypyridin-3-yl)-3-methyl-2-oxo-2,3-dihydro-1H-imidazo[4,5-c]quinolin-5-ium methanesulfonate, and
8-(6-Ammonio-5-(trifluoromethyl)pyridin-3-yl)-3-methyl-2-oxo-1-(6-(trifluoromethyl)pyridin-3-yl)-2,3-dihydro-1H-imidazo[4,5-c]quinolin-5-ium methanesulfonate,
or a stereoisomer, a tautomer or N-oxide thereof.

17. A pharmaceutical composition comprising a therapeutically effective amount of a compound of formula (I) as defined in claim 1 or its pharmaceutically acceptable salt and a pharmaceutically acceptable excipient or a carrier.

18. A method for the treatment of a disease or disorder mediated by one or more kinases selected from phosphatidylinositol 3 kinase (PI3K), mammalian target of rapamycin (mTOR), activin receptor-like kinase 1 (ALK1) or activin receptor-like kinase 2 (ALK2), comprising administering to a mammal in need thereof a therapeutically effective amount of a compound of formula (I) according to claim 1 or a pharmaceutically acceptable salt thereof.

19. The method according to claim 18, wherein the disease is a proliferative disease.

20. The method according to claim 19, wherein the proliferative disease is cancer.

21. The method according to claim 20, wherein the cancer is selected from leukemia, lung cancer, brain tumors, Hodgkin's disease, liver cancer, kidney cancer, bladder cancer, breast cancer, endometrial cancer, head and neck cancer, lymphoma, melanoma, cervical cancer, thyroid cancer, gastric cancer, germ cell tumor, cholangiocarcinoma, extracranial cancer, sarcoma, mesothelioma, malignant fibrous histiocytoma of bone, retinoblastoma, esophageal cancer, multiple myeloma, oral cancer, pancreatic cancer, neuroblastoma, skin cancer, ovarian cancer, recurrent ovarian cancer, prostate cancer, testicular cancer, colorectal cancer, lymphoproliferative disease, refractory multiple myeloma, cancer of urinary tract, resistant multiple myeloma or myeloproliferative disorder.

22. A method for the treatment of a disease mediated by tumor necrosis factor-α (TNF-α) or interleukin-6 (IL-6) comprising administering to a mammal in need thereof a therapeutically effective amount of a compound of formula (I) according to claim 1 or a pharmaceutically acceptable salt thereof.

23. The method according to claim 22, wherein the disease is an inflammatory disease.

24. The method according to claim 23, wherein the inflammatory disease is selected from rheumatoid arthritis, Crohn's disease, ulcerative colitis, inflammatory bowel disease, chronic non-rheumatoid arthritis, osteoporosis, septic shock, psoriasis or atherosclerosis.

25. A method for the treatment of a disease mediated by vascular endothelial growth factor (VEGF) comprising administering to a mammal in need thereof a therapeutically effective amount of a compound of formula (I) according to claim 1 or a pharmaceutically acceptable salt thereof.

26. The method according to claim 22, wherein the disease is angiogenesis related disorder.

27. The method according to claim 26, wherein the disease is: (i) an inflammatory disorder selected from immune and non-immune inflammation, chronic articular rheumatism, psoriasis, diabetic retinopathy, neovascular glaucoma, capillary proliferation in atherosclerotic plaques or osteoporosis; or (ii) cancer associated disorder selected from solid tumor, solid tumor metastases, angiofibroma, retrolental fibroplasia, hemangioma or Kaposi's sarcoma.

28. A method for the treatment of proliferative disease, inflammatory disease or an angiogenesis related disorder comprising administering to a mammal in need thereof a therapeutically effective amount of a compound of formula (I) according to claim 1 or a pharmaceutically acceptable salt thereof.

29. A process for the preparation of a compound of formula 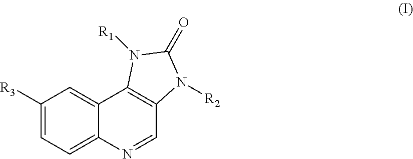
wherein R₁, and R₃ are as defined for formula (I) in claim 1,
comprising:
reacting a compound of formula (6); 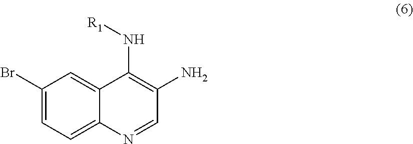
wherein, R₁ is as defined for formula (I) in claim 1,
with a reagent selected from trichloromethylchloroformate or triphosgene in the presence of a base selected from triethylamine or trimethylamine in a solvent selected from dichloromethane or chloroform to obtain a compound of formula (7); 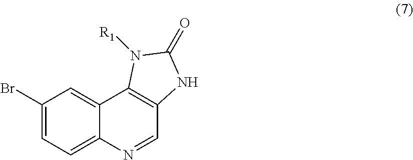
b) reacting the compound of formula (7), with a compound of formula R₂-hal, wherein, hal is halogen and R₂ is as defined for formula (I) in claim 1, in the presence of sodium hydride as a base, to obtain a compound of formula (8) 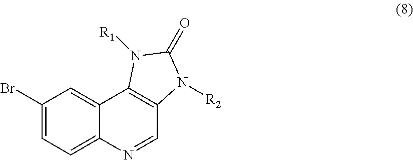
wherein R₁ and R₂ are as defined for formula (I) in claim 1;
(c) reacting the compound of formula (8) with a compound of formula R₃—B(OH)₂ wherein, R₃ is as defined for formula (I) in claim 1, in the presence of palladium dichlorobistriphenylphosphine as a coupling agent, to obtain the compound of formula (I), 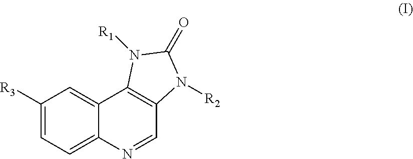
wherein R₁, R₂ and R₃ are as defined for formula (I);
d) optionally converting the resulting compound of formula (I) into a pharmaceutically acceptable salt.
